(19) United States
(12) Patent Application Publication
(10) Pub. No.: US 2022/0348638 A1
(43) Pub. Date: Nov. 3, 2022

(54) METHOD OF TARGETING NEURONAL APOE TO TREAT A NEUROCOGNITIVE DISORDER

(71) Applicant: The J. David Gladstone Institutes, a testamentary trust estabilished under the Will of J. David Glad, San Francisco, CA (US)

(72) Inventors: Yadong Huang, Danville, CA (US); Kelly Zalocusky, Mountain View, CA (US)

(21) Appl. No.: 17/763,424

(22) PCT Filed: Sep. 24, 2020
(86) PCT No.: PCT/US2020/052389

§ 371 Date: Mar. 24, 2022

Related U.S. Application Data
(60) Provisional application No. 62/905,101, filed on Sep. 24, 2019.

(51) Int. Cl. C07K 14/775; A61P 25/28

(52) U.S. Cl. CPC C07K 14/775; A61P 25/28

(57) ABSTRACT

A method for reducing neuronal and synaptic degeneration or loss in a population of neuronal cells is provided as well as a method of treating an individual with a neurocognitive disorder. Aspects of the methods include modulating the level and/or activity of apolipoprotein E (apoE) in a population of neuronal cells where the modulating reduces the level and/or activity of an MHC pathway polypeptide in the population of neuronal cells.

### PRIORITY

This application claims the benefit of priority from U.S. Provisional Patent Application Ser. No. 62/905,101, filed on Sep. 24, 2019, which is herein incorporated in its entirety by reference.

### STATEMENT REGARDING FEDERALLY SPONSORED RESEARCH

This invention was made with government support under Contract Numbers R01AG048017 and RF1AG055421, awarded by the National Institutes of Health, National Institute on Aging. The government has certain rights in the invention.

### INTRODUCTION

Across the diversity of neurodegenerative diseases, particular brain regions and cell types are especially vulnerable. In Parkinson's Disease, for example, dopamine neurons of the substantia nigra are disproportionately impacted, while nearby or even intermingled cells are spared (Poewe et al., 2017). Likewise, in Alzheimer's Disease (AD), there is a regional susceptibility in the hippocampus and entorhinal cortex, with a particular vulnerability in CA1 principal cells and hilar interneurons relative to other neuronal types (Andrews-Zwilling et al., 2010; Fu et al., 2018; Huang and Mucke, 2012; Mahley and Huang, 2012; Najm et al., 2019). Even within these susceptible neuronal populations, however, some cells are lost early while others prove more resilient.

AD is the most common form of dementia, a class of diseases estimated to affect 50 million people today and projected to affect 82 million people by 2030 (Patterson, 2018). The major genetic risk factor for AD is apolipoprotein E4 (apoE4), which both increases disease risk and decreases age of disease onset in carriers. Although apoE4 carriers account for only 20-25% of the general population, they represent 60-75% of AD cases, highlighting the importance of apoE4 in AD pathogenesis (Farrer et al., 1997; Huang and Mucke, 2012; Liu et al., 2013; Mahley and Huang, 2012; Ward et al., 2012). Within the central nervous system, the apoE protein is primarily produced in astrocytes (Pitas et al., 1987) but has been shown to be produced in neurons under conditions of stress, injury, and aging (Wadhwani et al., 2019; Wang et al., 2018; Xu et al., 1996, 1999, 2006). Neuronal apoE4 expression has been shown to diminish synaptic plasticity, impair synaptogenesis, and decrease synaptic density in both in vitro and in vivo systems (Brodbeck et al., 2011; Huang and Mucke, 2012; Lin et al., 2018; Najm et al., 2019; Wadhwani et al., 2019; Wang et al., 2018). Additionally, in a mouse model of tauopathy, human apoE expression—especially the apoE4 isoform—led to increased pathology, neuroinflammation, and neuronal loss, while apoE deficiency protected against these insults, suggesting a dose effect of apoE protein in addition to isoform-specific effects (Shi et al., 2017). Because apoE4 is a major genetic risk factor for AD, neurons produce the protein under stress and aging, and apoE dose-dependently exacerbates AD-related pathologies.

### SUMMARY

A method for reducing neuronal and synaptic degeneration or loss in a population of neuronal cells is provided, as well as a method of treating an individual with a neurocognitive disorder. Aspects of the methods include modulating the level and/or activity of apolipoprotein E (apoE) in a population of neuronal cells where the modulating reduces the level and/or activity of an MHC pathway polypeptide in the population of neuronal cells. Whether and how neuronal apoE expression may contribute to selective neuronal vulnerability in AD was tested by single cell analysis in both human apoE knock-in (apoE-KI) mouse and human brains

### BRIEF DESCRIPTION OF THE DRAWINGS

The invention may be best understood from the following detailed description when read in conjunction with the accompanying drawings. Included in the drawings are the following figures:

FIGS. 1A-C show the experimental design of mouse hippocampal single-nucleus RNA sequencing and cell cluster identification. (A) Experimental design. Hippocampi were extracted from apoE3-KI and apoE4-KI mice at 5, 10, 15, and 20 months of age (n=4 per genotype and age). The hippocampi were dissociated, nuclei were labeled with DAPI and isolated using flow cytometry before processing using the 10× Chromium v2 system for single-nucleus RNA sequencing. (B) Clustering using the Seurat package revealed 27 distinct cellular populations. Marker gene analysis led to the identification of 16 neuronal clusters and 11 non-neuronal clusters, including astrocytes, OPCs, oligodendrocytes, endothelial cells, and choroid plexus. (C) Violin plot depicting marker genes for larger cell classes (such as Syn1 for neurons) as well as marker genes for individual clusters, such as C1q12 for dentate gyms granule cells, Pdgfra for OPCs, and Fo1r1 for choroid plexus.

FIGS. 2A-F show that principal components analysis (PCA) reveals the most prominent sources of cell-by-cell variation within each cell type. Across multiple neuronal cell types, apoE mRNA levels correlate with the first two PCs, and that the top 10 pathways loadings onto the first 2 PCs are significantly enriched for MHC-related pathways. (A) In Cluster 2 DG cells (n=21550), apoE expression is strongly correlated with PC1 (r=0.81, p<2×10⁻¹⁶) and PC2 (r=0.37, p<2×10⁻¹⁶). The top 10 pathways loadings onto PC1 and PC2 are enriched for MHC-related pathways (bold; PC1 enrichment p<0.001; PC2 enrichment p<0.0001). (B) In Cluster 4 CA1 Pyramid cells (n=5684), apoE expression is strongly correlated with PC1 (r=−0.76, p<2×10⁻¹⁶) and PC2 (r=−0.61, p<2×10⁻¹⁶). The top 10 pathways loadings onto PC2 are enriched for MHC-related pathways (bold; PC2 enrichment p<0.01). (C) In Cluster 4 CA2/CA3 Pyramid cells (n=3488), apoE expression is strongly correlated with PC1 (r=0.82, p<2×10⁻¹⁶) and PC2 (r=0.19, p<2×10⁻¹⁶). The top 10 pathways loadings onto PC2 are significantly enriched for MHC-related pathways (bold; p=0.012). (D) In Cluster 9 Subiculum/Entorhinal neurons (n=2075), apoE expression is strongly correlated with PC1 (r=−0.84, p<2×10⁻¹⁶) and PC2 (r=0.41, p<2×10⁻¹⁶). The top 10 pathways loadings onto PC2 are enriched for MHC-related pathways (bold; p<0.001). (E) In Cluster 10 SST/PV interneurons (n=4801), apoE expression is strongly correlated with PC1 (r=0.77, p<2×10⁻¹⁶) and PC2 (r=0.11, p<2×10⁻¹⁶). The top 10 pathways loadings onto PC1 are enriched for MHC-related pathways (bold; p<0.001). (F) In Cluster 17 Astrocytes (n=2976), apoE expression is strongly correlated with PC1 (r=0.95, p<2×10⁻¹⁶) and PC2 (r=0.17, p<2×10⁻¹⁶). Unlike in neuronal clusters, there is no representation of immune or MHC-related pathways in the top 10 pathways loadings onto PC1 or PC2 for astrocytes, despite their relatively high level of baseline apoE expression relative to neurons. Instead, the top pathways loadings represent to biosynthesis, metabolism, and intercellular signaling pathways.

FIGS. 3A-D show the top correlates of neuronal apoE expression are enriched for MHC signaling pathways. (A) A direct examination of the 10 pathways most correlated with apoE expression in each neuronal cell type reveals an enrichment for MHC-related pathways (22%; enrichment test by bootstrap, p<0.01). Color represents Pearson's R between apoE expression and expression of each pathway for each cluster of cells. (B) A linear model to predict antigen processing and presentation score in each cell type, using apoE expression, age, genotype, age-by-genotype interaction, and sample number as covariates describes the percent of variance explained by each of those variables. Across neuronal clusters, apoE expression explains 47% (SST/PV interneurons) to 91% (CA1 pyramid cells) of the variation in antigen processing and presentation score. In contrast, in astrocytes, the relationship between apoE and antigen processing and presentation score is negligible. In all cases, the contributions of age, APOE genotype, age×APOE genotype interaction, and sample to this relationship, after accounting for apoE expression, are negligible. (C) Network visualization of the proportion of shared genes amongst the pathways represented in A. There are four main modules of inter-related pathways. The orange module is related to neurodegenerative disease and includes the Alzheimer disease, Huntington disease, and Parkinson disease pathways. The green module relates to cellular metabolism, and the blue module relates to DNA replication and repair. Strikingly, the largest module, consisting of nine apoE-correlated pathways, relates to MHC signaling (pink). Within the pink module, the antigen processing and presentation pathway is the most densely connected, with the largest edge sum. (D) ApoE expression correlates strongly with antigen processing and presentation score across many neuronal clusters, including DG granule cells (r²=0.82, p<1×10⁻³⁰⁰, n=21550), CA1 pyramid cells (r²=0.91, p<1×10⁻³⁰⁰ n=5684), CA2/CA3 pyramid cells (r²=0.81, p<1×10⁻³⁰⁰ n=3488), Subiculum/Entorhinal neurons (r²=0.83, p<1×10⁻³⁰⁰, n=2075), and SST/PV interneurons (r²=0.47, p<1×10⁻³⁰⁰, n=4801). This relationship does not extend to astrocytes, despite their relatively high baseline expression of apoE (r²=0.0004, p=0.30, n=2976).

FIGS. 4A-K show neuron-specific knockout of the APOE gene reduces MHC pathway expression and abolishes MHC pathways as major contributing factors to cell-by-cell variance of hippocampal neurons in apoE-KI mice. (A-D) In mice with the APOE gene knocked out of neurons, within-cell-type variability in dentate gyms granule cells (n=5578), CA1 pyramidal cells (n=2016), CA2/CA3 pyramidal cells (n=731), and SST/PV interneurons (n=462) is not enriched for immune or MHC-related pathways. (E) Clustering of the combined data from hippocampal single-nucleus RNA sequencing of 15-month-old ApoE-KI/Syn-Cre mice and 15-months-old ApoE-KI mice, clustered by cell type. (F) The datasets were successfully combined using CCA for batch correction. (G,H) ApoE expression is abolished specifically in neurons in ApoE-KI/Syn-Cre mice (H) relative to ApoE-KI mice (G). (I,J) Antigen processing and presentation score is substantially reduced across neuronal clusters in ApoE-KI/Syn-Cre mice (J) relative to ApoE-KI mice (I). (K) Genes from the antigen processing and presentation pathway that are differentially expressed in ApoE-KI/Syn-Cre neurons relative to ApoE-KI neurons. Color indicates log 2 fold change. Significance is BH-corrected q<0.05 by the non-parametric Wilcoxon rank-sum test. Only genes that are significantly differentially expressed in at least one cell type are shown.

FIGS. 5A-I show neuronal apoE expression correlates with MHC pathways in human brains. (A) Clustering of the dataset by cell type. (B) ApoE expression across cell types, demonstrating expression of apoE across neuronal types. (C) tSNE representation by donor (D) tSNE representation by cortical layer (E) tSNE representation by sex (F) Heatmap illustrating the correlation between apoE expression and KEGG pathway expression scores for the top 10 apoE expression-correlated pathways from each subset of neurons. Colors represent Pearson's r. Pathways shared with mouse data are highlighted in red. (G) Network plot illustrating the proportion of shared genes amongst apoE expression-correlated pathways shared between human and mouse. Edge width represents proportion of shared genes. There are two main modules of inter-related pathways. One is related to neurodegenerative disease and includes the Alzheimer disease and Huntington disease. The other module, consisting of eight apoE-correlated pathways, is related to MHC signaling. (H) Correlation of apoE expression and antigen processing and presentation pathway score in Layer 5/6 cells (r²=0.83, p<1×10⁻³⁰⁰, n=1719) and in SST Interneurons (r²=0.69 p=5×10⁻²³⁵, n=917). Color indicates the number of cells per hexagon. (I) Percent variance in antigen processing and presentation pathway score explained by apoE expression level and other covariates in Layer 1/2 neurons, Layer 5/6 neurons, and SST interneurons. ApoE expression and residual variability account for the vast majority of variance, with sex, age, and age*sex interaction each explaining <0.5% of the total variance.

FIGS. 6A-M show neuron-specific knockout of the APOE gene protects from apoE4-induced MHC upregulation and neuronal and hippocampal volume loss in aged apoE-KI mice (15-16 months). (A-D) Differences were noticed in both the median gene expression and the distribution of apoE expression across cell types. In dentate gyms granule cells (A) and CA1 principal cells (B), the median apoE expression is approximately 40% lower than that observed in SST/PV interneurons (C), and the median expression in SST/PV cells is less than half of that observed in astrocytes (D). Astrocytes exhibit a strong negative skew of apoE expression, with most cells expressing a high level of apoE mRNA and a select few with a low level of expression (D). In contrast, the neuronal cell types exhibited a marked positive skew, with most neurons expressing a low level of apoE and a select few cells expressing apoE at a much higher level (A-C). Red dashed lines indicate 2 SD above the median apoE expression for each cell type, the threshold for apoE-high cells. (E-G) Neurons are defined as apoE-expression-high if they express apoE mRNA at more than two standard deviations above the median expression (dashed red lines in A-C) for that cell type. The proportion of apoE-expression-high cells varies by age and genotype. In both DG granule cells (E) and CA1 pyramidal cells (F), apoE4-KI mice exhibit a rapid increase in the proportion of apoE-expression-high cells between 5 and 10 months before declining. In apoE3-KI mice, apoE-expression-high cell frequency peaks around 15 months, with a subsequent decline. In SST/PV interneurons (G), in both apoE3-KI and apoE4-KI mice, the highest levels of apoE-expression-high cells are at 5 months with subsequent decline. This decline is faster and larger in apoE4-KI than in apoE3-KI mice. The blue dashed line indicates the expected proportion of apoE-high cells if age and genotype had no effect on this proportion. Stars represent p-values in a Chi-square test of independence by age and genotype, comparing the observed number of apoE-expression-high cells to the number expected if age and genotype had no effect (df=7; *p<0.05, **p<0.01, ***p<0.001). (H) Astrocytes have no cells more than 2 SD above the median apoE expression. (I,J) Aged apoE4-KI mice have a significantly lower density of NeuN/DAPI double-positive cells in CA1, as compared to apoE3-KI mice (two-way ANOVA with Tukey's HSD, p=0.003). Neuron-specific apoE-KO rescues neuronal density in CA1 to apoE3-KI levels (p=0.001). n=11-12 per group in I. (K) The degree of neuronal loss correlates directly with MHC-I expression in CA1 neurons (Pearson's r=−0.59, p=<0.001). n=47. (L,M) Hippocampal volume is significantly lower in apoE4-KI mice as compared to apoE3-KI mice (two-way ANOVA with Tukey's HSD, p=0.004). ApoE4-KI hippocampal volume loss is significantly rescued by the neuron-specific knockout of apoE (p=0.0002). ApoE3-KI hippocampal volume is also enhanced by the neuron-specific knockout of apoE (p=0.006). n=4 per group in L.

FIGS. 7A-J show neuron-specific knockout of the APOE gene protects from apoE4-induced neuronal MHC increase and aggregation as well as synaptic loss in aged apoE-KI mice (15-16 months). (A) Representative immunostaining with PSD-95 (red), MHC-I (OX-18, green), or NeuN (blue) antibody in CA1 pyramidal cells of apoE3-KI, apoE3-KI/Syn-Cre, apoE4-KI, and apoE4-KI/Syn-Cre mice. White arrowhead indicates PSD aggregates; yellow arrowhead indicates colocalized MHC aggregates. Scale bars=35 μm. (B) PSD-95 intensity in CA1 cell bodies is significantly lower in apoE4-KI than in apoE3-KI (two-way ANOVA, Tukey's HSD, p=0.007) or apoE4-KI/Syn-Cre (p<0.001) mice. n=11-12 per group. (C) PSD-95 intensity in CA1 dendrites is significantly lower in apoE4-KI than in apoE3-KI (two-way ANOVA, Tukey's HSD, p=0.039) or apoE4-KI/Syn-Cre (p=0.006) mice. n=11-12 per group. (D) Mean PSD-95 aggregates per cell is significantly higher in apoE4-KI than in apoE3-KI (two-way ANOVA, Tukey's HSD, p<0.001) or apoE4-KI/Syn-Cre (p<0.001) mice. n=11-12 per group. (E) PSD intensity in CA1 dendrites is significantly inversely correlated with the number of PSD-95 aggregates per cell (Pearson's r=−0.44, p=0.002). n=47. (F) The percent of PSD-95 aggregate area that colocalizes with MHC-I is significantly higher in apoE4-KI than in apoE4-KI/Syn-Cre (two-way ANOVA, Tukey's HSD, p=0.003) mice and in apoE3-KI than in apoE3-KI/Syn-Cre (p<0.001) mice. n=11-12 per group. (G) Mean number of MHC puncta per CA1 pyramidal cell significantly correlates with the number of PSD-95 aggregates per cell (Pearson's r=0.045, p=0.001). n=47. (H) The intensity of MHC-I staining in the CA1 pyramidal layer is significantly higher in apoE4-KI than in apoE3-KI (two-way ANOVA, Tukey's HSD, p<0.001) or apoE4-KI/Syn-Cre (p<0.001) mice. n=11-12 per group. (I) The average number of MHC-I puncta per cell in the CA1 pyramidal layer is significantly higher in apoE4-KI than in apoE3-KI (two-way ANOVA, Tukey's HSD, p<0.001) or apoE4-KI/Syn-Cre (p=0.009) mice. It is also significantly higher in apoE4-KI/Syn-Cre than in apoE3-KI/Syn-Cre (p<0.001) mice. n=11-12 per group. *p<0.05, **p<0.01, ***p<0.001 (J) Model of apoE upregulation of MHC driving selective neuronal and synaptic degeneration/loss. Neurons under stress from aging, injury, excitotoxicity, or other insults upregulate their expression of apoE. ApoE expression in stressed neurons drives expression of MHC, as shown at both the RNA and protein level. ApoE and MHC in concert drive selective neuronal and synaptic degeneration/loss. Neuronal MHC expression drives neuronal and synaptic degeneration/loss, likely through serving as an “eat me” signal to resident microglia or infiltrating T-cells.

FIGS. 8A-C show cell cluster identification and quality control of single-nucleus

RNA sequencing analysis of apoE-KI mice. (A) Feature plots of imputed expression of marker genes for major cell type clusters, as well as matched whole-brain and hippocampal expression of that marker gene in the Allen Institute for Brain Science Mouse ISH Atlas (Lein et al., 2007). (B) tSNE plots of all the nuclei broken out by apoE genotype (rows) and mouse age (columns) showing a lack of batch effect by sample and representation of all major cell types in both genotypes at all ages. (C) Quality control measures: number of UMIs, number of genes, and percent mitochondrial reads from each cluster.

FIGS. 9A-L show ApoE correlation with the first two PCs is not driven by age, genotype, cell type markers, or quality control markers. (A-D) PCA plots demonstrating that the correlation between apoE gene expression and the first 2 principal components (PC1 and PC2) across neuronal cell types is not driven by measures of quality control or read depth, such as number of UMIs, number of genes, or percent mitochondrial reads. (E-H) Neither is the apoE expression gradient driven by apoE genotype or mouse age. (I-L) Additionally, this apoE expression gradient is not explained by differences in cell type marker expression, such as Syn1 for neurons or Aqp4 for astrocytes (I-L), indicating that the apoE-expression-high cells are not misclassified neuron/astrocyte doublets.

FIGS. 10A-B show ApoE and pathway correlations are highly similar across age and apoE genotype. (A) Heatmaps showing apoE and pathway correlation across cell types for 12 pathways of interest, broken out by apoE genotype and mouse age, demonstrating a strong conservation of apoE and pathway relationships across genotypes and ages. (B) ApoE and antigen processing correlations in CA1 pyramidal cells, broken out by age and apoE genotype, demonstrating a strong conservation of apoE and pathway relationships across genotypes and ages.

FIG. 11 shows ApoE expression correlations with individual genes in the antigen processing and presentation pathway in apoE-KI mice. For each cell type, the heatmap represents the strength and direction of correlation between apoE expression and the expression of each individual gene in the antigen processing and presentation pathway in apoE-KI mice.

FIGS. 12A-B show cell cluster identification and apoE expression in the combined set of apoE-KI and apoE-KI/Syn-Cre data. (A) Feature plots of marker genes for major cell types in the combined apoE-KI and apoE-KI/Syn-Cre cell clustering. (B) Histograms of apoE expression levels in the combined apoE-KI and apoE-KI/Syn-Cre cohort, showing that even the low levels of apoE expression measured in apoE-KI neurons are true expression, fully separated from the noise levels in apoE-KI/Syn-Cre neurons.

### DETAILED DESCRIPTION

A method for reducing neuronal and synaptic degeneration or loss in a population of neuronal cells is provided as well as a method of treating an individual with a neurocognitive disorder. Aspects of the methods include modulating the level and/or activity of apolipoprotein E (apoE) in a population of neuronal cells where the modulating reduces the level and/or activity of an MI-1C pathway polypeptide in the population of neuronal cells.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a membrane anchored polynucleotide” includes a plurality of such membrane-anchored polynucleotides and reference to “the polynucleotide” includes reference to one or more polynucleotides, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention.

Further, the dates of publication provided may be different from the actual publication dates which may need to be independently confirmed. To the extent such publications may set out definitions of a term that conflicts with the explicit or implicit definition of the present disclosure, the definition of the present disclosure controls.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

As used herein, the term “neurodegeneration” refers broadly to a defect involving or relating to the nervous system. As used herein, the terms “neurodegenerative disorder” or “neurodegenerative disease” refer broadly to disorders or diseases that affect the nervous system, including but are not limited to Parkinson's disease, Alzheimer's disease, Huntington's disease and amyotrophic lateral sclerosis. As used herein, the term “neurodegeneration is reduced” refers to the improvement in the neurodegenerative condition, such that the degree of neurodegeneration is lessened.

As used herein, the term “subject suffering from a neurodegenerative disease” refers to both humans and animals displaying symptoms normally associated with a disease that affects the nervous system. As used herein, the term “animals” refers to all non-human animals. Such non-human animals include, but are not limited to, vertebrates such as rodents (e.g., rats), non-human primates, ovines, bovines, lagomorphs, porcines, caprines, equines, canines, felines, ayes, etc.

The term “neurocognitive disorders” is used herein as a synonym of “neurocognitive diseases” and includes, but is not limited to, Alzheimer's Disease (AD) which is the main representative example of all related dementias and neurocognitive disorders. References to AD may therefore be equally taken as references to Mild Cognitive Impairment (MCI) (a recognised precursor to AD) and other late onset dementias including vascular dementia, dementia with lewy bodies and fronto-temporal dementia, alone and as a mixed dementia with Alzheimer's disease, unless it is explicitly specified the progression between MCI and AD. It may also refer to a specific diagnosis given to a subject or it may also include symptoms of that neurocognitive disorders where a specific diagnosis has not been yet formalised by a medical practitioner according to the present clinical assessment measures. Currently, the disease status is assessed by duration of disease from inception to present (longer duration equals more severe disease) and clinical assessment measures. These assessment measures include clinical tests for memory and other cognitions, clinical tests for function (abilities of daily living) and clinical assessments of global severity. Trials of potential therapies in AD and other dementias and neurocognitive disorders are currently evaluated against these measures. The FDA and other regulatory authorities require as part of these assessments measures of both cognition and global function. The Global Dementia Scale is one such measure of global function. It is assessed by assessment of severity including cognition and function against a standardized set of severity criteria.

The term “Alzheimer's disease” (abbreviated herein as “AD”) as used herein refers to a condition associated with formation of neuritic plaques comprising amyloid 13 protein primarily in the hippocampus and cerebral cortex, as well as impairment in both learning and memory. “AD” as used herein is meant to encompass both AD as well as AD-type pathologies.

As used herein, “Mild Cognitive Impairment” or “MCI” refers to a condition characterized by isolated memory impairment unaccompanied other cognitive abnormalities and relatively normal functional abilities. One set of criteria for a clinical characterization of MCI specifies the following characteristics: (1) memory complaint (as reported by patient, informant, or physician), (2) normal activities of daily living (ADLs), (3) normal global cognitive function, (4) abnormal memory for age (defined as scoring more than 1.5 standard deviations below the mean for a given age), and (5) absence of indicators of dementia (as defined by DSM-IV guidelines). Petersen et al., Srch. Neurol. 56: 303-308 (1999); Petersen, “Mild cognitive impairment: Aging to Alzheimer's Disease.” Oxford University Press, N.Y. (2003).

The term “apoE” refers to apolipoprotein E, a 34,000 molecular weight protein that is the product of a single gene on chromosome 19 and exists in three major isoforms designated apoE2, apoE3 and apoE4. ApoE mRNA is abundant in the brain, where it is synthesized and secreted primarily by astrocytes. Although apoE is synthesized in the brain primarily by astrocytes, neurons in the central nervous system (CNS) express apoE in response to excitotoxic stress and other insults. It has been shown that neuronal expression of apoE, especially apoE4, contributes to the pathogenesis of Alzheimer's Disease (AD), such as neurofibrillary tangle formation and mitochondrial dysfunction. The apoE4 allele is a major risk factor or susceptibility gene associated with approximately 40-65% of cases of sporadic and familial Alzheimer's disease and it increases the occurrence and lowers the age of onset of the disease.

As used herein, an “apoE-associated disorder” or an “apoE-related disorder” is any disorder that is caused by the presence of apoE (K00396.1) (e.g., apoE3 (reference sequence; human; NM_001302689.2 (nucleic acid and amino acid sequence)) or apoE4 (reference sequence; human; NM_001302690.2 (nucleic acid and amino acid sequence))) in a cell, in the serum, in the interstitial fluid, in the cerebrospinal fluid, or in any other bodily fluid of an individual; any disorder that is characterized by the presence of apoE3 or apoE4; a symptom of a disorder that is caused by the presence of apoE3 or apoE4 in a cell or in a bodily fluid; a phenomenon associated with a disorder caused by the presence in a cell or in a bodily fluid of apoE3 or apoE4; and the sequelae of any disorder that is caused by the presence of apoE3 or apoE4. ApoE-associated disorders include apoE-associated neurological disorders and disorders related to high serum lipid levels. ApoE-associated neurological disorders include, but are not limited to, sporadic Alzheimer's disease; familial Alzheimer's disease; poor outcome following a stroke; poor outcome following traumatic head injury; and cerebral ischemia. Phenomena associated with apoE-associated neurological disorders include, but are not limited to, neurofibrillary tangles; amyloid deposits; memory loss; and a reduction in cognitive function. ApoE-related disorders associated with high serum lipid levels include, but are not limited to, atherosclerosis, and coronary artery disease. Phenomena associated with such apoE-associated disorders include high serum cholesterol levels.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” used interchangeably herein, refer to a mammal, including, but not limited to, humans; and non-human mammals, e.g., murines, simians, mammalian farm animals, mammalian sport animals, and mammalian pets.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the term “neurons,” or “neuronal cells” includes any cell population that includes neurons of any type, including, but not limited to, primary cultures of brain cells that contain neurons, isolated cell cultures comprising primary neuronal cells, neuronal precursor cells, tissue culture cells that are used as models of neurons, and mixtures thereof.

The term “PSD-95” as used herein refers to postsynaptic density protein-95. PSD-95 is encoded by the DLG4 gene and is a member of the membrane-associated guanylate kinase (MAGUK) family comprising PSD-95, PSD-93, SAP102 (synapse-associated protein-102), and SAP97, which share three conserved PDZ domains and one SH3-GK (Src homology 3-guanylate kinase) module. PSD-95 is the major scaffolding protein in the excitatory postsynaptic density (PSD). The PSD-95 family MAGUKs play prominent roles in synaptic plasticity.

As used herein, the term “major histocompatibility complex (MHC) polypeptides” is meant to include MHC polypeptides of various species, including human MHC (also referred to as human leukocyte antigen (HLA)) polypeptides, rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. The term “MHC polypeptide” is meant to include Class I MI-IC polypeptides (e.g., β-2 microglobulin and MHC class I heavy chain) and MHC Class II polypeptides (e.g., MHC Class II a polypeptide and MHC Class II β polypeptide).

In a class I-restricted immune response, MHC class I molecules are associated with peptide antigens derived from proteins made intracellularly. Such proteins include proteins encoded by viruses and other intracellular pathogens. These proteins are degraded in the cytoplasm of infected cells, and the peptide products of this degradation transferred into the endoplasmic reticulum (ER) via the action of peptide-specific transporter molecules located in the ER membrane (see, Elliott et al., 1990, Nature 348: 195-197; Parham, 1990, Nature 348: 674-675). Nascent MHC class I molecules synthesized in the ER are assembled into functional presenting proteins only in the presence of the appropriate peptide antigen. Fully assembled MHC class I complexes are then transported through the Golgi apparatus to the cell surface, where the antigen presenting complex can activate a cellular (T-cell mediated) immune response by interacting with CD8⁺ cytotoxic T-cells (see, Falk et al., 1990. Nature 348: 248-251; Falk et al., 1991, Nature 351: 290-296).

In a MHC class II restricted immune response, extracellular antigens (including free-living pathogens or protein components thereof) are engulfed by cells of the immune system (such as macrophages) by endocytosis, and transferred to the endosomal (lysosomal) compartment for degradation. Peptide products of such degradation may then associate with MHC class II molecules (which molecules lack the requirement of peptide association for cell-surface expression; see, Germain & Hendrix, 1991, Nature 353: 134-139) and appear on the cell surface (see, Sadegh-Nasseri & Germain, 1991, Nature 353: 167-170; Lanzavecchia et al., 1992, Nature 357: 249-252). The MHC class II antigen-presenting pathway leads to the induction of a humoral (antibody-dependent) immune response and the activation of CD4⁺ T-helper cells.

The terms “MHC class I antigen presentation pathway”, “MHC class II antigen presentation pathway”, “major histocompatibility complex (MHC) Class I antigen processing and presentation pathway” or “major histocompatibility complex (MHC) Class II antigen processing and presentation pathway” refers to any gene and products thereof involved in the processing or presenting of antigenic peptides on MHC class I or class II molecules. Genes involved in the pathways include, but are not limited to, components of MHC class I molecules, components of MHC class II molecules, components of the peptide-loading complex, and components of the immuno-proteosome.

### Methods

The present disclosure provides methods for reducing neuronal and synaptic degeneration or loss in a population of neuronal cells as well as methods for treating an individual with a neurocognitive disorder. Various steps and aspects of the methods will now be described in greater detail below.

### Method for Reducing Neuronal and Synaptic Degeneration or Loss

As described above, methods of the present disclosure include a method for reducing neuronal and synaptic degeneration or loss in a population of neuronal cells. The method may include modulating the level and/or activity of apolipoprotein E (apoE) in the population of neuronal cells, wherein the modulating reduces the level and/or activity of at least one MHC pathway polypeptide in the population of neuronal cells compared to the level and/or activity of the at least one MHC pathway polypeptide in a population of neuronal cells in the absence of said modulating. As used herein, the term “level” may refer to an amount of protein or transcript (e.g., apoE or apoE mRNA) present in a cell at a given time. As used herein, the term “activity” may refer to one or more functions, e.g., lipid transport, etc., of apoE.

In some cases, the apoE of the subject methods is an isoform of apoE. In some cases, the apoE present in the population of neuronal cells is apoE4. In some cases, the apoE present in the population of neuronal cells is apoE3.

The method may include modulating the level and/or activity of apoE by any suitable amount. As used herein, the terms “modulating the level and/or activity of apoE” may refer to increasing or decreasing the level and/or activity of apoE in a population of neuronal cells compared to the level and/or activity of apoE in a population of neuronal cells in the absence of said modulating. In some cases, the modulating includes inhibiting the level (e.g., expression) and/or activity of apoE. In some cases, the method includes modulating the level and/or activity of apoE in each neuronal cell of a population of neuronal cells. In some cases, the method includes modulating the level and/or activity of apoE by an amount effective to modulate the level and/or activity of an MHC pathway polypeptide in the population of neuronal cells. In some cases, the method includes modulating the level and/or activity of apoE by an amount effective to decrease the level and/or activity of an MHC pathway polypeptide in the population of neuronal cells. In some cases, the method includes reducing the level and/or activity of apoE by an amount effective to decrease the level/activity of an MHC pathway polypeptide in the population of neuronal cells. In some cases, the method includes modulating the level and/or activity of apoE by 1 to 10-fold, 1 to 5-fold, or 1 to 3-fold. In some cases, the method includes modulating the level and/or activity of apoE by at least 5%, at least 10%, at least 15%, at least 20%, at least 30%, at least 40%, at least 50%, at least 60%, at least 70%, at least 80%, at least 90%, or at least 95%. In some cases, the method includes modulating the level and/or activity of apoE by an amount ranging from 5% to 50%, from 5% to 40%, from 5% to 30%, from 5% to 20%, from 5% to 15%, from 5% to 10%. In some cases, the method includes modulating the level and/or activity of apoE by an amount ranging from 10% to 90%, from 10% to 80%, from 10% to 70%, from 10% to 60%, from 10% to 50%, from 10% to 40%, from 10% to 40%, from 10% to 30%, or from 10% to 20%. In some cases, the method includes modulating the level and/or activity of apoE by an amount ranging from 10% to 90%, from 20% to 90%, from 30% to 90%, from 40% to 90%, from 50% to 90%, from 60% to 90%, from 70% to 90%, or from 80% to 90%. In some cases, the method includes modulating the level and/or activity of apoE by an amount ranging from 50% to 95%, from 50% to 90%, from 50% to 80%, from 50% to 75%, from 50% to 60%, or from 50% to 55%. In some cases, the method includes modulating the level and/or activity of apoE by an amount ranging from 30% to 40%, from 50% to 60%, or from 60% to 70%.

The level and/or activity of apoE may be modulated by any suitable means. In some cases, the level and/or activity of apoE may be modulated by contacting the population of neuronal cells with an agent. Suitable agents include, e.g., antibodies, small molecules, protein inhibitors, siRNA, viral vectors among others. In some cases, the level and/or activity of apoE is modulated by a method of gene editing in the population of neuronal cells. In some cases, the level and/or activity of apoE is modulated by contacting the population of neuronal cells with a nuclease (e.g., ZFN, TALEN, RNA-guided endonuclease, genome editing nuclease, etc.). In some cases, the level and/or activity of apoE is modulated by contacting the population of neuronal cells with a complex including a CRISPR/Cas effector polypeptide and a guide RNA. The contacting may occur under conditions suitable for a reaction to occur, e.g., for enzymatic cleavage to occur, as described in, e.g., simplified CRISPR tools for efficient genome editing and streamlined protocols for their delivery into mammalian cells and mouse zygotes. Methods, 121-122, 16-28. doi:10.1016/j.ymeth.2017.03.021. A CRISPR enzyme suitable for inclusion in the methods of the present disclosure includes an RNA-guided endonuclease. The CRISPR enzyme may be a Class 2 CRISPR effector protein, also referred to herein as a class 2 CRISPR/Cas effector polypeptide. Examples of RNA-guided endonucleases are CRISPR/Cas endonucleases (e.g., class 2 CRISPR/Cas endonucleases such as a type II, type V, or type VI CRISPR/Cas endonucleases). A suitable genome editing nuclease is a CRISPR/Cas endonuclease (e.g., a class 2 CRISPR/Cas endonuclease such as a type II, type V, or type VI CRISPR/Cas endonuclease). In some cases, a suitable RNA-guided endonuclease is a class 2 CRISPR/Cas endonuclease. In some cases, a suitable RNA-guided endonuclease is a class 2 type II CRISPR/Cas endonuclease (e.g., a Cas9 protein). In some cases, a genome targeting composition includes a class 2 type V CRISPR/Cas endonuclease (e.g., a Cpf1 protein, a C2c1 protein, or a C2c3 protein). In some cases, a suitable RNA-guided endonuclease is a class 2 type VI CRISPR/Cas endonuclease (e.g., a C2c2 protein; also referred to as a “Cas13a” protein). Also suitable for use is a CasX protein. Also suitable for use is a CasY protein.

In some cases, the modulating reduces MI-IC signaling by the population of neuronal cells. The reduction in MHC signaling may include reducing signaling by the population of neuronal cells to immune cells and/or microglia. The reducing of MHC signaling may include reducing the level and/or activity of at least one MHC pathway polypeptide, as described below. The reducing of MHC signaling may include reducing the amount of an MHC class I molecule and/or an MHC class II molecule expressed by the population of neuronal cells. In some cases, the reducing of MHC signaling includes reducing the activity of an MHC class I molecule or an MHC class II molecule expressed by the population of neuronal cells. In some cases, reducing MHC signaling by the population of neuronal cells reduces neuronal and synaptic degeneration or loss in the population.

In some cases, the level and/or activity of the MHC pathway polypeptide may be modulated by any suitable amount, e.g., an amount effective to reduce neuronal and synaptic degeneration or loss. As described above, the level or amount of the at least one MHC pathway polypeptide may be modulated by the presence, level, and/or activity of apoE. In certain embodiments, modulating the level and/or activity of apoE modulates the level and/or activity of the at least one MHC pathway polypeptide, e.g., in each neuronal cell of a population of neuronal cells. In certain embodiments, modulating the level and/or activity of apoE decreases the level and/or activity of the at least one MHC pathway polypeptide. In some cases, decreasing the level and/or activity of apoE decreases the level and/or activity of the at least one MHC pathway polypeptide. In some cases, modulating the level and/or activity of apoE decreases the level and/or activity of the at least one MHC pathway polypeptide in a population of neuronal cells present in a brain region of an individual. In some cases, the brain region is the hippocampus. In some cases, the level and/or activity of the at last one MHC pathway polypeptide decreases 1 to 10-fold, 1 to 5-fold, or 1 to 3-fold. In some cases, the level and/or activity of the at least one MHC pathway polypeptide decreases by at least 5%, at least 10%, at least 15%, at least 20%, at least 30%, at least 40%, at least 50%, at least 60%, at least 70%, at least 80%, at least 90%, or at least 95%. In some cases, the level and/or activity of the at least one MHC pathway polypeptide decreases by an amount ranging from 5% to 50%, from 5% to 40%, from 5% to 30%, from 5% to 20%, from 5% to 15%, from 5% to 10%. In some cases, the level and/or activity of the at least one MHC pathway polypeptide decreases by an amount ranging from 10% to 90%, from 10% to 80%, from 10% to 70%, from 10% to 60%, from 10% to 50%, from 10% to 40%, from 10% to 40%, from 10% to 30%, or from 10% to 20%. In some cases, the level and/or activity of the at least one MHC pathway polypeptide decreases by an amount ranging from 10% to 90%, from 20% to 90%, from 30% to 90%, from 40% to 90%, from 50% to 90%, from 60% to 90%, from 70% to 90%, or from 80% to 90%. In some cases, the level and/or activity of the at least one MHC pathway polypeptide decreases by an amount ranging from 50% to 95%, from 50% to 90%, from 50% to 80%, from 50% to 75%, from 50% to 60%, or from 50% to 55%. In some cases, the level and/or activity of the at least one MHC pathway polypeptide decreases by an amount ranging from 30% to 40%, from 50% to 60%, or from 60% to 70%.

The at least one MHC pathway polypeptide may include a molecule associated with antigen processing and presentation, e.g., an antigen processing and presentation polypeptide. In some cases, the at least one MHC pathway polypeptide includes a polypeptide associated with an MHC signaling pathway. In some cases, the at least one MHC pathway polypeptide is a polypeptide involved in the major histocompatibility complex (MHC) Class I antigen processing and presentation pathway or major histocompatibility complex (MHC) Class II antigen processing and presentation pathway. In some cases, the at least one MHC pathway polypeptide includes Tapbp. In some cases, the at least one MHC pathway polypeptide includes Tap2. In some cases, the at least one MHC pathway polypeptide includes MHC polypeptides. In some cases, the at least one MHC pathway polypeptide includes β2m. In some cases, the at least one MHC pathway polypeptide includes MHC Class I. In some cases, the at least one MHC pathway polypeptide includes MHC Class II. In some cases, the at least one MHC pathway polypeptide includes a polypeptide encoded by an MHC class I gene including, e.g., HLA-A, HLA-B, HLA-C, HLA-E, HLA-F, HLA-G. In some cases, the at least one MHC pathway polypeptide comprises a polypeptide encoded by an MHC class II gene including, e.g., HLA-DPA, HLA-DRA, HLA-DRB1.

In some cases, the population of neuronal cells includes one or more neuronal cell types. In some cases, the population neuronal cells is a population of neuronal ells in brain region. In some cases, the population of neuronal cells are derived from the hippocampus. In some cases, the population of neuronal cells comprises at least one of excitatory neurons, inhibitory neurons, or interneurons. In some cases, the population of neuronal cells comprises at least one of dentate gyms granule cells, CA1 principal cells, CA2/CA3 principal cells, subiculum/entorhinal cells, or SST/PV interneurons. In some cases, the population of neuronal cells includes human cortical neurons.

In certain aspects, the population of neuronal cells is a population of neuronal cells in brain region of an individual. In certain aspects, the population of neuronal cells has been subjected to at least one of stress, injury, and aging. In some cases, the individual has a neurocognitive disorder, neurodegenerative disorder, or a stage thereof including, but not limited to, mild cognitive impairment, prodromal Alzheimer's Disease, Alzheimer's Disease, Huntington's disease, Parkinson's disease, etc.

In some cases, the modulating the level and/or activity of apoE is effective for treating the neurocognitive disorder or stages thereof in the individual. In some cases, the level and/or activity of apoE is modulated by an amount effective to treat the neurocognitive disorder or stages thereof in the individual. In some cases, the modulating of the level and/or activity of apoE may reduce one or more indicators or symptoms of the neurocognitive disorder or stages thereof in the individual.

In some cases, the method includes modulating the level and/or activity of apoE by an amount effective to reduce loss of neuronal density in the population of neuronal cells. In some cases, the population of neuronal cells is a population of neuronal cells in a brain region of an individual. In some cases, the brain region is the hippocampus or a region of the hippocampus. In some cases, the modulating reduces loss of hippocampal neuronal density in the individual. The neuronal density may refer to the number of neurons per unit area (e.g., cells per mm²) in the brain region of an individual. In some cases, the method includes decreasing the level and/or activity of apoE by an amount effective to reduce loss of neuronal density. In some cases, the modulating reduces loss of neuronal density by at least 5%, at least 10%, at least 15%, at least 20%, at least 30%, at least 40%, at least 50%, at least 60%, at least 70%, at least 80%, at least 90%, or at least 95%. In some cases, the modulating reduces loss of neuronal density by an amount ranging from 5% to 50%, from 5% to 40%, from 5% to 30%, from 5% to 20%, from 5% to 15%, from 5% to 10%. In some cases, the modulating reduces loss of neuronal density by an amount ranging from 10% to 90%, from 10% to 80%, from 10% to 70%, from 10% to 60%, from 10% to 50%, from 10% to 40%, from 10% to 40%, from 10% to 30%, or from 10% to 20%. In some cases, the modulating reduces loss of neuronal density by an amount ranging from 10% to 90%, from 20% to 90%, from 30% to 90%, from 40% to 90%, from 50% to 90%, from 60% to 90%, from 70% to 90%, or from 80% to 90%. In some cases, the modulating reduces loss of neuronal density by an amount ranging from 50% to 95%, from 50% to 90%, from 50% to 80%, from 50% to 75%, from 50% to 60%, or from 50% to 55%. In some cases, the modulating reduces loss of neuronal density by an amount ranging from 30% to 40%, from 50% to 60%, or from 60% to 70%.

In some cases, the method includes modulating the level and/or activity of apoE by an amount effective to reduce loss of volume in a population of neuronal cells. In some cases, the population of neuronal cells is a population of neuronal cells in a brain region (e.g., measured in mm³) in an individual. In some cases, the brain region is the hippocampus or a region of the hippocampus. In some cases, the modulating reduces loss of hippocampal volume in the individual. In some cases, the method includes decreasing the level and/or activity of apoE by an amount effective to reduce loss of volume in a brain region. In some cases, the modulating reduces loss of volume in a brain region by at least 5%, at least 10%, at least 15%, at least 20%, at least 30%, at least 40%, at least 50%, at least 60%, at least 70%, at least 80%, at least 90%, or at least 95%. In some cases, the modulating reduces loss of volume in a brain region by an amount ranging from 5% to 50%, from 5% to 40%, from 5% to 30%, from 5% to 20%, from 5% to 15%, from 5% to 10%. In some cases, the modulating reduces loss of volume in a brain region by an amount ranging from 10% to 90%, from 10% to 80%, from 10% to 70%, from 10% to 60%, from 10% to 50%, from 10% to 40%, from 10% to 40%, from 10% to 30%, or from 10% to 20%. In some cases, the modulating reduces loss of volume in a brain region by an amount ranging from 10% to 90%, from 20% to 90%, from 30% to 90%, from 40% to 90%, from 50% to 90%, from 60% to 90%, from 70% to 90%, or from 80% to 90%. In some cases, the modulating reduces loss of volume in a brain region by an amount ranging from 50% to 95%, from 50% to 90%, from 50% to 80%, from 50% to 75%, from 50% to 60%, or from 50% to 55%. In some cases, the modulating reduces loss of volume in a brain region by an amount ranging from 30% to 40%, from 50% to 60%, or from 60% to 70%.

In some cases, the method includes modulating the level and/or activity of apoE by an amount effective to reduce synaptic loss in the population of neuronal cells. In some cases, the population of neuronal cells is a population of neuronal cells in a brain region of the individual. In some cases, the modulating reduces synaptic loss in the hippocampus or a region of the hippocampus in the individual. In some cases, modulating synaptic loss includes modulating PSD-95 intensity, an amount of PSD-95 aggregates, and colocalization of PSD-95 aggregates with at least one MHC pathway polypeptide in a population of neuronal cells.

In some cases, the modulating reduces a decline in PSD-95 intensity in the population of neuronal cells. In some cases, the modulating reduces a decline in PSD-95 intensity in each neuronal cell of the population. In some cases, the modulating reduces a decline in PSD-95 intensity by at least 5%, at least 10%, at least 15%, at least 20%, at least 30%, at least 40%, at least 50%, at least 60%, at least 70%, at least 80%, at least 90%, or at least 95%. In some cases, the modulating reduces a decline in PSD-95 intensity by an amount ranging from 5% to 50%, from 5% to 40%, from 5% to 30%, from 5% to 20%, from 5% to 15%, from 5% to 10%. In some cases, the modulating reduces a decline in PSD-95 intensity by an amount ranging from 10% to 90%, from 10% to 80%, from 10% to 70%, from 10% to 60%, from 10% to 50%, from 10% to 40%, from 10% to 40%, from 10% to 30%, or from 10% to 20%. In some cases, the modulating reduces a decline in PSD-95 intensity by an amount ranging from 10% to 90%, from 20% to 90%, from 30% to 90%, from 40% to 90%, from 50% to 90%, from 60% to 90%, from 70% to 90%, or from 80% to 90%. In some cases, the modulating reduces a decline in PSD-95 intensity by an amount ranging from 50% to 95%, from 50% to 90%, from 50% to 80%, from 50% to 75%, from 50% to 60%, or from 50% to 55%. In some cases, the modulating reduces a decline in PSD-95 intensity by an amount ranging from 30% to 40%, from 50% to 60%, or from 60% to 70%.

In some cases, the modulating reduces PSD-95 aggregates in the population of neuronal cells. In some cases, the modulating reduces PSD-95 aggregates in each neuronal cell of the population. In some cases, the modulating reduces PSD-95 aggregates by at least 5%, at least 10%, at least 15%, at least 20%, at least 30%, at least 40%, at least 50%, at least 60%, at least 70%, at least 80%, at least 90%, or at least 95%. In some cases, the modulating reduces PSD-95 aggregates by an amount ranging from 5% to 50%, from 5% to 40%, from 5% to 30%, from 5% to 20%, from 5% to 15%, from 5% to 10%. In some cases, the modulating reduces PSD-95 aggregates by an amount ranging from 10% to 90%, from 10% to 80%, from 10% to 70%, from 10% to 60%, from 10% to 50%, from 10% to 40%, from 10% to 40%, from 10% to 30%, or from 10% to 20%. In some cases, the modulating reduces PSD-95 aggregates by an amount ranging from 10% to 90%, from 20% to 90%, from 30% to 90%, from 40% to 90%, from 50% to 90%, from 60% to 90%, from 70% to 90%, or from 80% to 90%. In some cases, the modulating reduces PSD-95 aggregates by an amount ranging from 50% to 95%, from 50% to 90%, from 50% to 80%, from 50% to 75%, from 50% to 60%, or from 50% to 55%. In some cases, the modulating reduces PSD-95 aggregates by an amount ranging from 30% to 40%, from 50% to 60%, or from 60% to 70%.

In some cases, the modulating reduces colocalization of PSD-95 aggregates with an MHC pathway polypeptide, e.g., an MHC class I molecule, in the population of neuronal cells. In some cases, the modulating reduces colocalization of PSD-95 aggregates with an MHC pathway polypeptide in each neuronal cell of the population. In some cases, the modulating reduces colocalization of PSD-95 aggregates with an MHC pathway polypeptide by at least 5%, at least 10%, at least 15%, at least 20%, at least 30%, at least 40%, at least 50%, at least 60%, at least 70%, at least 80%, at least 90%, or at least 95%. In some cases, the modulating reduces colocalization of PSD-95 aggregates with an MHC pathway polypeptide by an amount ranging from 5% to 50%, from 5% to 40%, from 5% to 30%, from 5% to 20%, from 5% to 15%, from 5% to 10%. In some cases, the modulating reduces colocalization of PSD-95 aggregates with a MHC pathway polypeptide by an amount ranging from 10% to 90%, from 10% to 80%, from 10% to 70%, from 10% to 60%, from 10% to 50%, from 10% to 40%, from 10% to 40%, from 10% to 30%, or from 10% to 20%. In some cases, the modulating reduces colocalization of PSD-95 aggregates with a MHC pathway polypeptide by an amount ranging from 10% to 90%, from 20% to 90%, from 30% to 90%, from 40% to 90%, from 50% to 90%, from 60% to 90%, from 70% to 90%, or from 80% to 90%. In some cases, the modulating reduces colocalization of PSD-95 aggregates with an MHC pathway polypeptide by an amount ranging from 50% to 95%, from 50% to 90%, from 50% to 80%, from 50% to 75%, from 50% to 60%, or from 50% to 55%. In some cases, the modulating reduces colocalization of PSD-95 aggregates with an MHC pathway polypeptide by an amount ranging from 30% to 40%, from 50% to 60%, or from 60% to 70%.

In some cases, the modulating reduces the percent of PSD-95 aggregate area that colocalizes with an MHC pathway polypeptide, e.g., an MHC class I molecule, in the population of neuronal cells. In some cases, the modulating reduces the percent of PSD-95 aggregate area that colocalizes with a MHC pathway polypeptide by at least 5%, at least 10%, at least 15%, at least 20%, at least 30%, at least 40%, at least 50%, at least 60%, at least 70%, at least 80%, at least 90%, or at least 95%. In some cases, the modulating reduces the percent of PSD-95 aggregate area that colocalizes with an MHC pathway polypeptide by an amount ranging from 5% to 50%, from 5% to 40%, from 5% to 30%, from 5% to 20%, from 5% to 15%, from 5% to 10%. In some cases, the modulating reduces the percent of PSD-95 aggregate area that colocalizes with a MHC pathway polypeptide by an amount ranging from 10% to 90%, from 10% to 80%, from 10% to 70%, from 10% to 60%, from 10% to 50%, from 10% to 40%, from 10% to 40%, from 10% to 30%, or from 10% to 20%. In some cases, the modulating reduces the percent of PSD-95 aggregate area that colocalizes with a MHC pathway polypeptide by an amount ranging from 10% to 90%, from 20% to 90%, from 30% to 90%, from 40% to 90%, from 50% to 90%, from 60% to 90%, from 70% to 90%, or from 80% to 90%. In some cases, the modulating reduces the percent of PSD-95 aggregate area that colocalizes with an MHC pathway polypeptide by an amount ranging from 50% to 95%, from 50% to 90%, from 50% to 80%, from 50% to 75%, from 50% to 60%, or from 50% to 55%. In some cases, the modulating reduces the percent of PSD-95 aggregate area that colocalizes with an MHC pathway polypeptide by an amount ranging from 30% to 40%, from 50% to 60%, or from 60% to 70%.

### Method of Treating an Individual

As described above, in another aspect, methods of the present disclosure include a method of treating an individual with a neurocognitive disorder or stages thereof including, e.g., MCI, prodromal Alzheimer's Disease, and Alzheimer's Disease. The method includes modulating the level and/or activity of apolipoprotein E (apoE) in a population of neuronal cells of the individual, wherein the modulating reduces the level and/or activity of at least one MHC pathway polypeptide in the population of neuronal cells compared to the level and/or activity of the at least one MHC pathway polypeptide in the population of neuronal cells in the absence of said modulating, and wherein the modulating is effective for treating the neurocognitive disorder or stages thereof in the individual. The population of neuronal cells may include any type of neuronal cell, as described above. The step of modulating may be performed according to any of the embodiments described in the present disclosure.

In some cases, the apoE of the subject methods is an isoform of apoE. In some cases, the apoE present in the population of neuronal cells is apoE4. In some cases, the apoE present in the population of neuronal cells is apoE3.

In some cases, the modulating reduces the level and/or activity of apoE by any suitable amount, e.g., any of the amounts described above. The level and/or activity of apoE may be modulated by any suitable means, as described above.

In some cases, the modulating reduces MHC signaling by the population of neuronal cells. In certain embodiments, the modulating reduces the level and/or activity of the at least one MHC pathway polypeptide by any suitable amount, e.g., any of the amounts described above. The at least one MHC pathway polypeptide may be any of the MEW pathway polypeptides described above including, e.g., Tapbp, Tap2, β2m, MHC class I, or MHC class II.

In some cases, the modulating reduces loss of neuronal density, reduces loss of volume, reduces synaptic loss, reduces a decline in PSD-95 intensity, reduces PSD-95 aggregates, and/or reduces colocalization of PSD-95 aggregates with at least one MHC pathway polypeptide in the population of neuronal cells by any suitable amount, as described above.

A variety of subjects are suitable for treatment with a subject method. Suitable subjects include any individual, particularly a human, who has an apoE-associated disorder, who is at risk for developing an apoE-associated disorder, who has had an apoE-associated disorder and is at risk for recurrence of the apoE-associated disorder, or who is recovering from an apoE-associated disorder. Such subjects include, but are not limited to, individuals who have been diagnosed as having mild cognitive impairment (MCI), prodromal Alzheimer's Disease, or Alzheimer's disease; individuals who have suffered one or more strokes; individuals who have suffered traumatic head injury; individuals who have A13 deposits in brain tissue; individuals who have had one or more cardiac events; subjects undergoing cardiac surgery; subjects with Parkinson's disease; subjects with amyotrophic lateral sclerosis; and subjects with multiple sclerosis.

In certain embodiments, the method further includes administering an effective amount of a therapeutic composition to the individual. An “effective amount” of a therapeutic agent means a dosage sufficient to produce a desired result, e.g., an improvement in learning, memory, a reduction in Aβ levels, a reduction in neuronal cell death, etc. The therapeutic composition may include an agent, as described above. In some cases, the level and/or activity of apoE is modulated by a method of gene editing by any suitable means as described above or known in the art. In some cases, the therapeutic agent can be formulated and/or modified to enable the agent to cross the blood-brain barrier. In some embodiments, the therapeutic composition decreases the level and/or activity of apoE in the population of neuronal cells. The therapeutic composition may decrease the level and/or activity of apoE by any amount as described above. In some cases, the therapeutic composition completely inhibits the expression and/or activity of apoE.

In certain aspects, the method further comprises detecting the level and/or activity of at least one MHC-1 pathway polypeptide in the population of neuronal cells by any suitable means known in the art.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for.

### Example 1

Selective neuronal degeneration is a critical causal factor in Alzheimer's Disease (AD); however, the mechanisms that lead some neurons to perish while others remain resilient are an enduring mystery to the field. ApoE4 is the major genetic risk factor for AD, and neurons express apoE under conditions of stress, injury, and aging. Using a single-nucleus RNA sequencing approach, it is found that 7-10% of various types of neurons in the hippocampus of human apoE knock-in (apoE-KI) mice express apoE at a high level. This expression is age-dependent, and apoE4-KI mice exhibit increased neuronal apoE expression at an earlier age than do apoE3-KI mice. Strikingly, neuronal apoE expression correlates strongly with major histocompatibility complex (MHC) pathways on a neuron-by-neuron basis in both AD mouse and human brains. In mice, neuron-specific apoE4 knock-out decreases neuronal MHC expression, increases synaptic density, and rescues neuronal and hippocampal volume loss. Thus, neuronal apoE, especially apoE4, upregulates MHC pathways to drive selective neurodegeneration, providing a window into both neuron-by-neuron differences in vulnerability and potential targets for preventing selective neurodegeneration in AD.

### Experimental Model and Subject Details

Mice

All protocols and procedures followed the guidelines of the Laboratory Animal Resource Center at the University of California, San Francisco (UCSF). Experimental and control mice had identical housing conditions from birth through sacrifice (12 h light/dark cycle, housed 5 animals/cage, PicoLab Rodent Diet 20). ApoE3-KI and apoE4-KI homozygous mouse lines (Taconic) (Hamanaka et al., 2000) were born and aged under normal conditions at the Gladstone Institute/UCSF animal facility. ApoE4-KI/Syn-Cre and apoE3-KI/Syn-Cre mice were generated by cross-breeding apoE-floxed-KI mice, which was generated in the lab (Bien-Ly et al., 2012), with Syn-Cre mice (Knoferle et al., 2014).

### Materials and Methods

Single-Nuclei Preparation for 10× Loading

To isolate single nuclei from adult mouse brains, the 10× Genomics demonstrated protocol for nuclei isolation from adult mouse brain (10× Genomics, 2018) and the Allen Brain Institute protocol for FACS sorting of single nuclei (Allen Institute for Brain Science, 2018) were combined and adapted as follows. Hippocampi were acutely dissected on ice. Dissected hippocampi were placed in 2 mL. Hibernate A®/B27®/GlutaMAX™ (HEB) medium in a 5 mL tube. The HEB medium was removed to a 15 mL conical and kept on ice. 2 mL of chilled lysis buffer (10 mM Tris-HCl, 10 mM NaCl, 3 mM MgCl2, and 0.1% Nonidet™ P40 Substitute in Nuclease-Free Water) were added to the tissue, and the hippocampi were homogenized by suctioning 10 times through a 21 G needle. After homogenization, the tissue was lysed on ice for 15 min, swirling 2-3 times during this incubation period. The reserved chilled HEB media was then returned to the lysed tissue solution, and the tissue was further triturated with 5-7 passes through a 1 mL pipette. A 30 μm cell strainer (MACS SmartStrainer; Miltenyi Biotech 130-110-915) was washed with 1 mL of PBS, and the lysed tissue solution was filtered through the strainer to remove debris and clumps. Filtered nuclei were centrifuged at 500 rcf for 5 min at 4° C. The supernatant was removed, and nuclei were washed in 1 mL of Nuclei Wash and Resuspension Buffer (1× PBS with 1.0% BSA and 0.2 U/μl RNase Inhibitor). Nuclei were again centrifuged at 500 rcf for 5 min at 4° C. and resuspended in 400 μL of Nuclei Wash and Resuspension Buffer. DAPI was added to a final concentration of 0.1 ug/mL, and the nuclei were filtered through a 35 μm cell strainer. DAPI-positive nuclei were sorted by gating on DAPI-positive events, excluding debris and doublets, using the BD FACSAria-II at the Gladstone Institutes' Flow Cytometry Core.

cDNA Library Preparation and Sequencing

cDNA libraries were prepared using the Chromium Single Cell 3′ Library and Gel Bead kit v2 (10× Genomics: 120267) according to the manufacturer's instructions. Libraries were sequenced on an Illumina NovaSeq 6000 sequencer at the UCSF CAT Core.

Pre-Processing and Clustering of Mouse Single-Nucleus RNA Sequencing Samples

The samples were aligned with Cellranger v.2.0.1 to a custom reference genome built from mm10-1.2.0 that includes introns, as nuclear pre-mRNA includes intronic portions. Each of the filtered UMI count matrices was loaded into Seurat v.2.3.4. Data were filtered to include only protein coding genes. Cells were filtered to include only cells with 200-2,400 genes detected, 500-4,500 UMI, and <0.25% mitochondrial reads. This quality assurance process resulted in a final matrix of 21,204 genes by 123,489 nuclei. The gene expression matrices were then log-normalized with a scale of factor of 10,000. Highly variable genes were selected by filtering for an average expression range of 0.25 to 4 and a minimum dispersion of 0.55, resulting in a list of 2,197 genes. Principal components (PCs) were calculated and visually examined as an elbow plot. The shared-nearest neighbor graph was constructed using the first 15 PCs and a resolution of 0.6, resulting in a set of 23 distinct clusters. Visualization of clusters was performed with a t-stochastic neighbors embedding (tSNE), again using the first 15 PCs.

Cell-Type Assignment

Data visualization by tSNE revealed clusters where mouse ages and genotypes were intermingled, with no discernable evidence of batch effects by genotype or age. Marker genes for each cluster were calculated using the FindAllMarkers function in Seurat (Butler et al., 2018; Stuart et al., 2019), using the Wilcoxon rank sum test, with the parameters log fc.threshold=0.25 and min.pct=0.1. Broad cell classes, such as excitatory and inhibitory neurons, astrocytes, oligodendrocytes, and OPCs were identified on the basis of canonical markers, and markers derived from previous RNA sequencing data on sorted cell types (Zhang et al., 2014). For further subdivision of hippocampal cell types, particularly for identification of subsets of principal cells, enrichment for genes identified in the hipposeq resource was used (Cembrowski et al., 2016). For rarer non-neuronal cell types, gene expression in the cells was compared to those genes enriched in each hippocampal cell type relative to all other cells in the hippocampus, according to the data in the DropViz resource (Saunders et al., 2018). To verify cell identity, the marker genes were additionally queried for each cluster against the Allen Brain's genome-wide atlas of gene expression in the adult mouse brain (Lein et al., 2007).

Human Single-Nucleus RNA Sequencing Data from the Allen Institute for Brain Science

The medial temporal gyms gene-counts-by-cell matrix and metadata were downloaded directly from the Allen Brain Institute's webpage (https://celltypes.brain-map.org/rnaseq). Thorough documentation on sample preparation can be found at (http://help.brain-map.org/display/celltypes/Documentation). From there, analysis follows the pipeline described elsewhere in this Methods section, including clustering with Seurat, cell type assignment, imputation with MAGIC, assignment of KEGG pathway scores, and correlations of KEGG pathway scores with APOE expression on a cell-by-cell basis.

Immunohistochemistry

30 μm coronal hemi-brain sections were washed 2×10 min in phosphate-buffered saline (PBS) then exposed to UV light overnight to reduce autofluorescence. The next day, slices were washed 3×10 min in PBS, then washed 2×15 min in PBS+0.1% Tween-20 (PBS-T). Slices were blocked in PBS+10% Normal Donkey Serum+0.2% Gelatin (Sigma)+0.5% Triton-X for 1 hr at room temperature (RT), then washed for 10 min in PBS. Because some antibodies were raised in mouse, slices were additionally blocked in 1 drop Mouse IgG Blocking Reagent (Vector Labs, MKB-2213-1) per 5 mL PBS for 1 hour at RT. Primary antibodies were diluted to optimized concentrations (anti-NEUN 1:1000, anti-OX18 1:50, anti PSD-95 1:200) in 1:12.5 mouse-on-mouse Protein Concentrate (Vector Labs, MKB-2213-1) in PBS, and slices were incubated overnight at 4° C. The next day, slices were washed 3× (15 min, 10 min, 5 min) in PBS-T. Secondary antibodies (Lifetech, Jackson ImmunoResearch; 1:1000) were diluted in the same dilution buffer as primary antibodies and incubated 1 hr at RT. Slices were then washed 3× (15 min, 10 min, 5 min) with PBS, mounted with Vectashield+DAPI, and coverslipped. Details about all reagents can be found in the Key Resources Table.

Image Analysis

Confocal fluorescent images (z-stacks) were acquired using a Zeiss LSM880 Confocal microscope. At least 3 z-stacks in at least 4 animals were analyzed for each brain region of interest for each genotype of mouse. Image analysis was performed using custom macros written in the open source Fiji (ImageJ) software. All analyses of PSD-95 intensity, PSD-95 aggregates, MHC-I intensity and MHC-I aggregates were performed in a fully automated fashion using custom macros to blind the analysis and exclude the possibility of bias. Analyses of hippocampal volume and of NeuN/DAPI+ cellular density were conducted manually (DeVos et al., 2017; Shi et al., 2017), but blind to sample, again to exclude the possibility of bias.

### Quantification and Statistical Analyses

Where applicable, all statistical details of the experiments including tests used, value of n, definition of center, and dispersion measures can be found in the corresponding figure legend. Additional description of statistical methods used is detailed on a per-experiment basis below.

MAGIC Imputation for Gene-Gene Interaction

The raw counts data were filtered to include only genes with >100 reads across all nuclei, library size and square-root normalized as described in (Dijk et al., 2018). Data were then imputed with the magic function, allowing the function itself to set the optimal parameters.

KEGG Pathway Scores

Pathway scores for individual nuclei were calculated within each cell type by subsetting the imputed gene×nuclei matrix to the nuclei within each defined cell type cluster and the genes within each Kyoto Encyclopedia of Genes and Genomes (KEGG) pathway. For each pathway, each gene's expression across nuclei within a cell type was normalized to the maximum expression of that gene in that cell type. In this way, each gene's normalized expression score would lie between 0 and 1, and the pathway score would not be dominated by the value of one or a few high-expressing genes. The normalized gene expression values for each gene were then averaged across all genes in the pathway to give a pathway expression score for each pathway for each nucleus.

Determination of Enrichment of Immune or MHC Pathways

Within the KEGG hierarchy of pathways are 328 total pathways divided into seven major subsets, each with their own sub-classification of pathways. These include 1. Metabolism, 2. Genetic Information Processing, 3. Environmental Information Processing, 4. Cellular

Processes, 5. Organismal Systems, 6. Human Diseases, and 7. Drug Development. Any pathway categorized as 5.1 Immune system, 6.3 Immune disease, 6.8 Infectious disease: bacterial, 6.9 Infectious disease: viral, or 6.10 Infectious disease: parasitic was considered an “immune related” pathway. In total there were 56 immune-related pathways. Any pathway containing HLA genes was considered an “MHC related” pathway. In total there were 18 MHC pathways. Simple bootstrapping (10,000 replicates) was used to determine the likelihood of finding at least as many MHC or immune-related pathways in a set of the size that was observed, given the proportions in the overall space of KEGG pathways.

ApoE-Pathway Correlation Heatmaps

For every cell type, the imputed apoE expression value for each individual cell was correlated with the pathway scores for every KEGG pathway for each individual cell. This gives a list of correlation values for each cell type for each pathway. For each neuronal cell type, the top 10 most-correlated pathways was selected. Any duplicated pathways were removed. This leaves a cell type×pathway matrix representing only those pathways most correlated with apoE expression in each cell type. These were then hierarchically clustered and displayed using the heatmap.2 function in the gplots package of R.

Gene Expression Network Analysis

Proportion of shared genes in KEGG pathways was calculated using custom software in R, with KEGG annotations from the limma package (Ritchie et al., 2015). Network visualizations were conducted in Cytoscape (Shannon et al., 2003).

Percent Variance Explained

A linear model that predicted the antigen processing and presentation score on a cell by cell basis was defined to be: pathway score˜apoE expression+age+apoE genotype+age*apoE genotype+sample ID.

Percent variance explained by each variable was calculated as sum of squares between groups over sum of squares total (η²).

Batch Correction with CCA

To directly compare the apoE-KI and apoe-KI/Syn-Cre datasets, it was necessary to combine the data objects using a batch correction method. The canonical correlation analysis (CCA) method built in to the Seurat package was used. An object only combining the aged Syn-Cre animals (15 months) with the 15-month-old apoE-KI animals was made. The 15 months apoE-KI animal dataset was further subset by a factor of 2 in order to approximately match the number of cells between the apoE-KI dataset and the apoE-KI/Syn-Cre dataset. Alignment was done based on the top 1000 most dispersed genes in each dataset. Cells were filtered out where the variance explained by CCA was <2-fold compared to PCA, then aligned on the first 20 CCs.

Percent apoE-Expression-High Cells

For each cell type, apoE-expression-high cells were those where the imputed apoE expression value was >=2 standard deviations above the median apoE expression value for that cell type, across all genotypes and ages. The population was then divided based on age and genotype to determine what proportion of each cell type, at each age, in each genotype was considered “apoE-expression-high”. Because this represents a single value, significance was determined using a chi-square test comparing the actual proportions of apoE-expression-high cells by age and genotype against the expected if age and genotype each had no effect on the proportion of apoE-expression-high cells.

Groupwise Statistics on Immunohistochemical Analysis

All statistics comparing apoE-KI to apoE-KI/Syn-Cre and apoE3-KI to apoE4-KI animals were conducted as a two-way ANOVA (y˜apoe genotype*Syn-Cre). P values displayed are from Tukey's Honest Significant Difference family-wise error corrected post-hoc tests.

### Results

Single-Nucleus RNA Sequencing Analysis of the Hippocampus in Human apoE Knock-In (apoE-KI) Mice

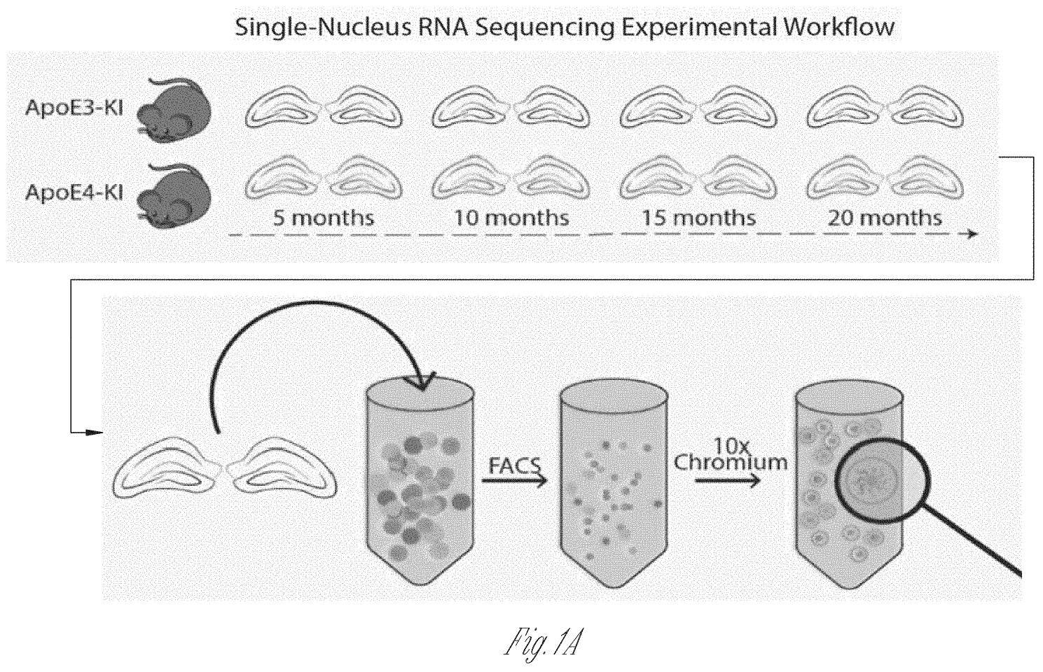

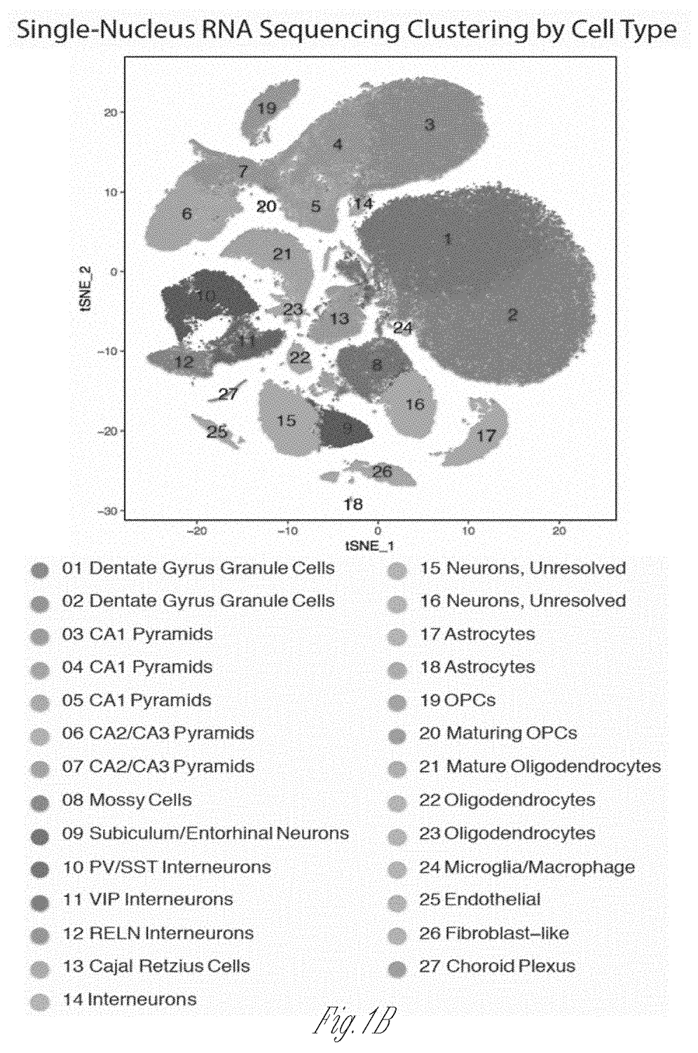

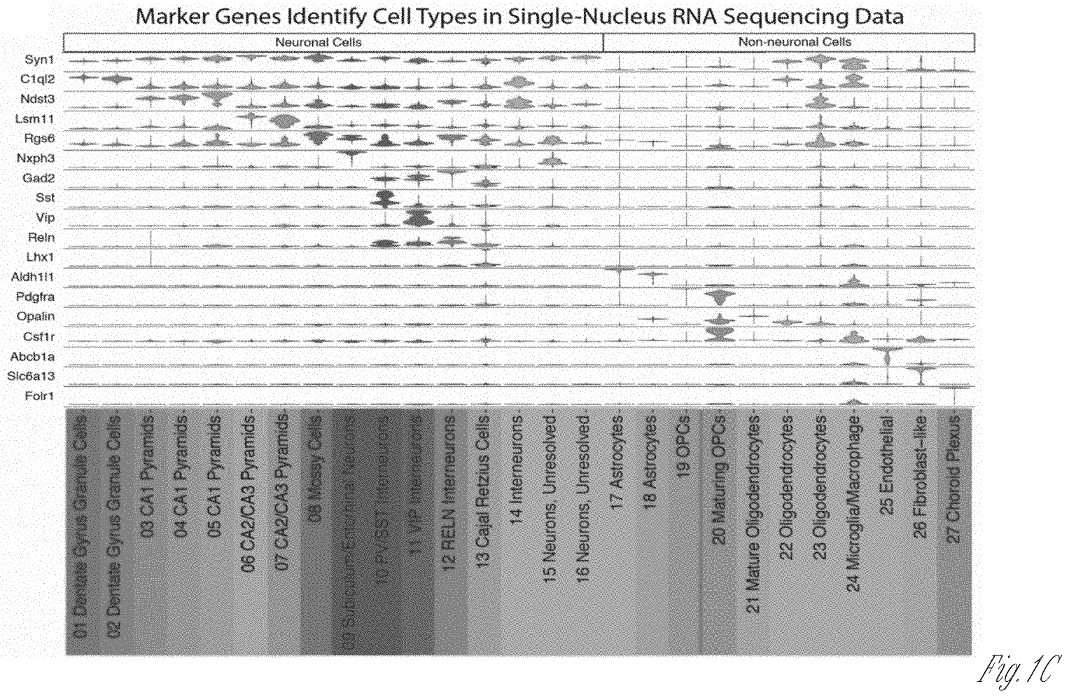

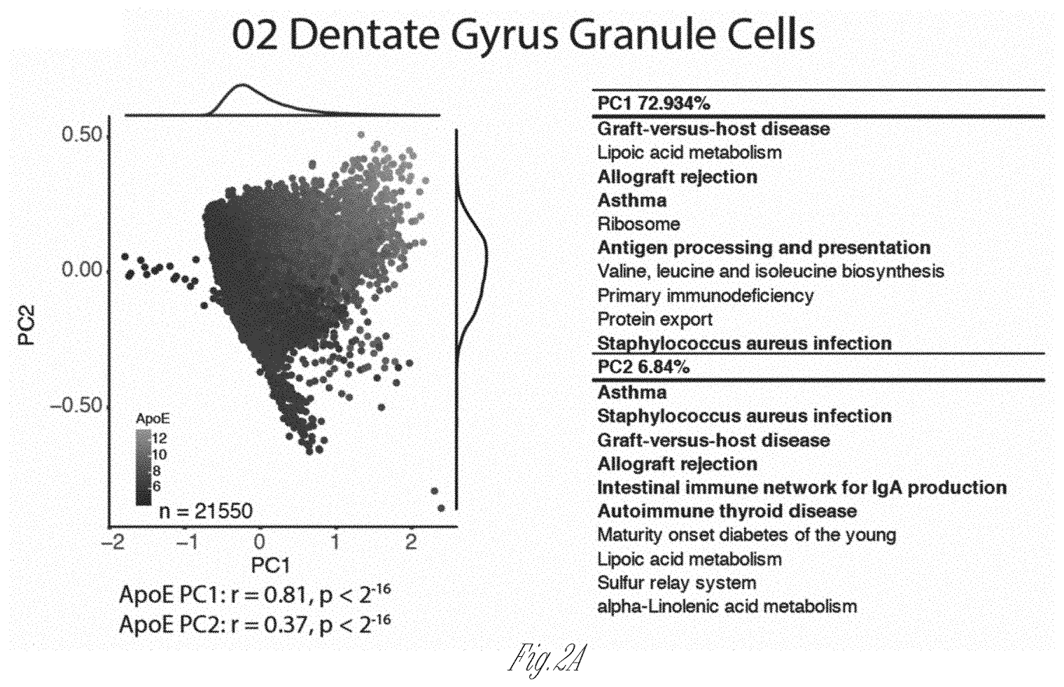

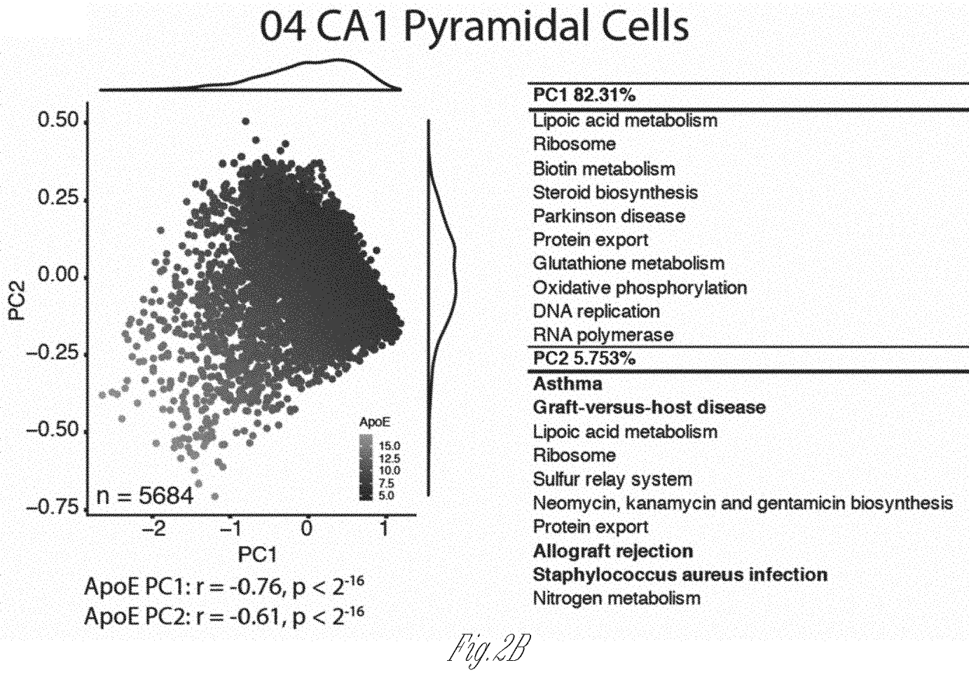

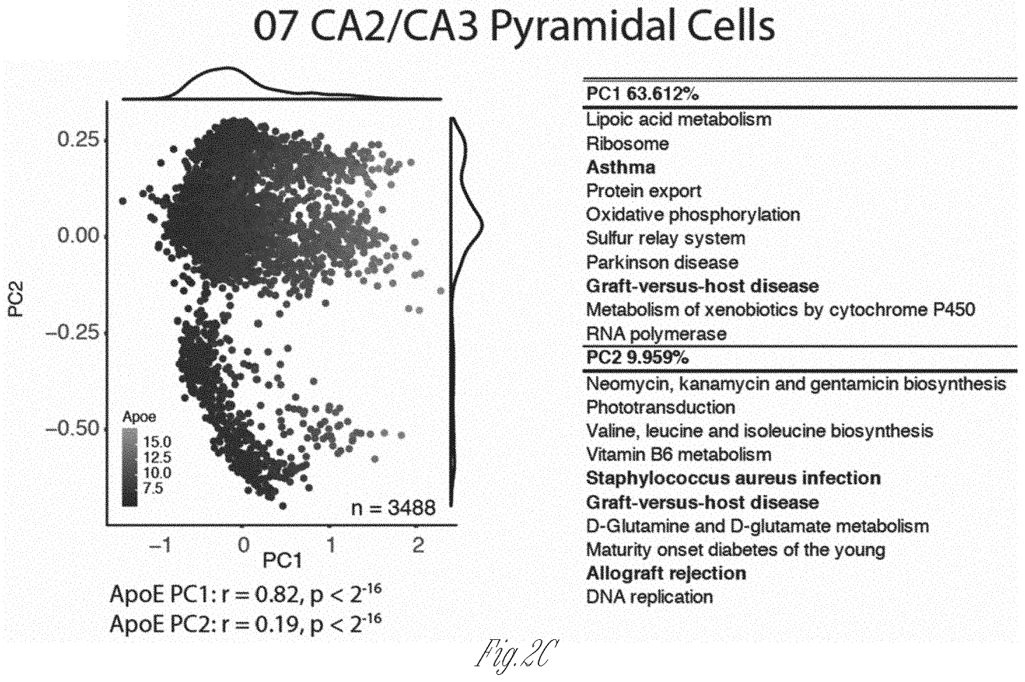

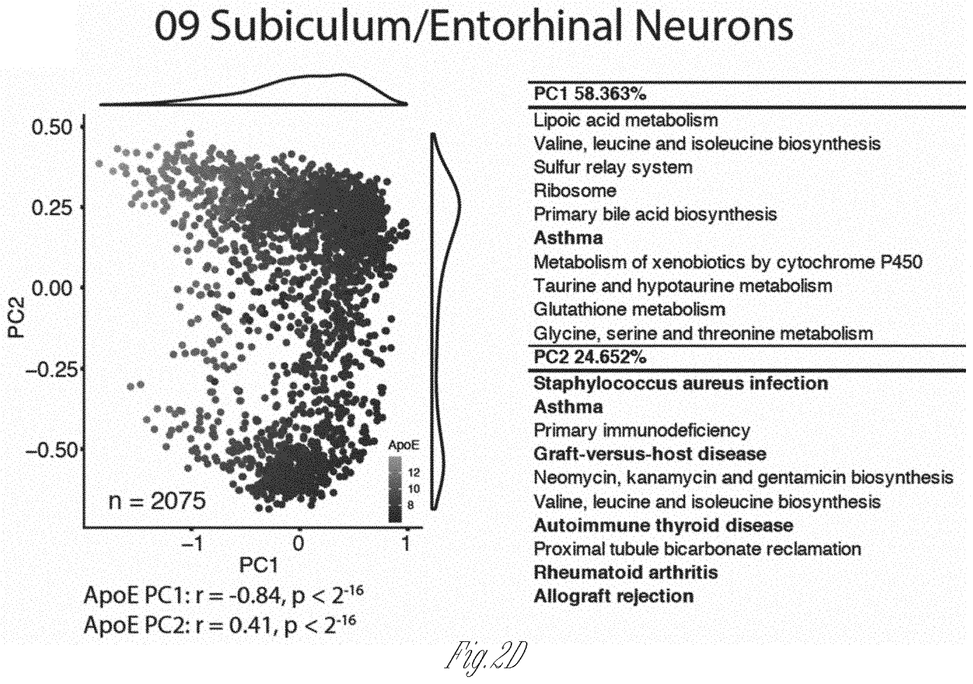

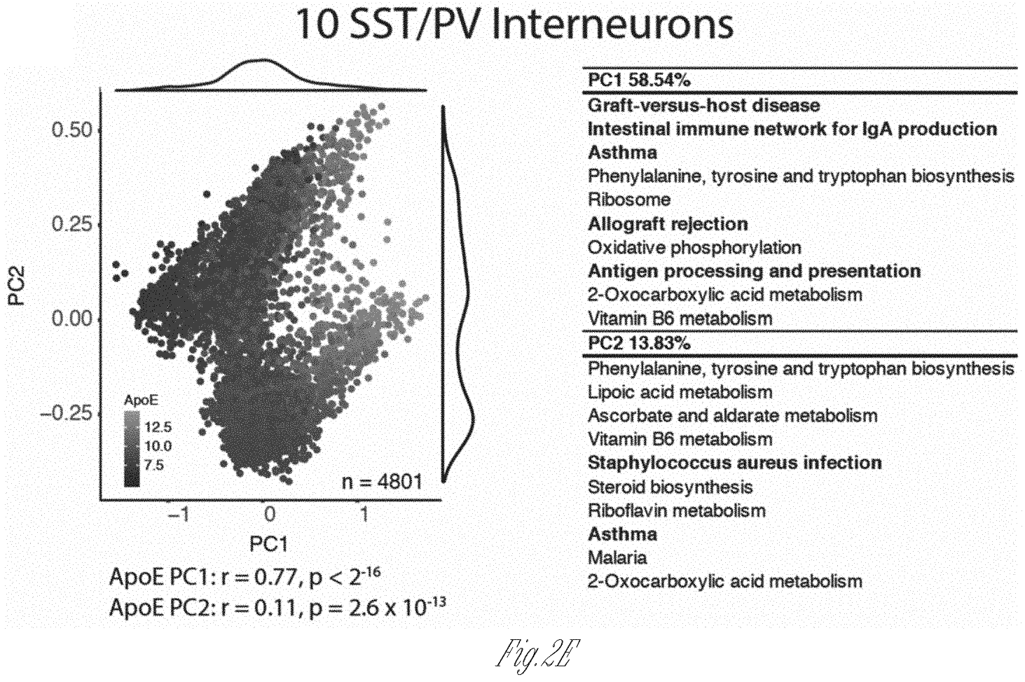

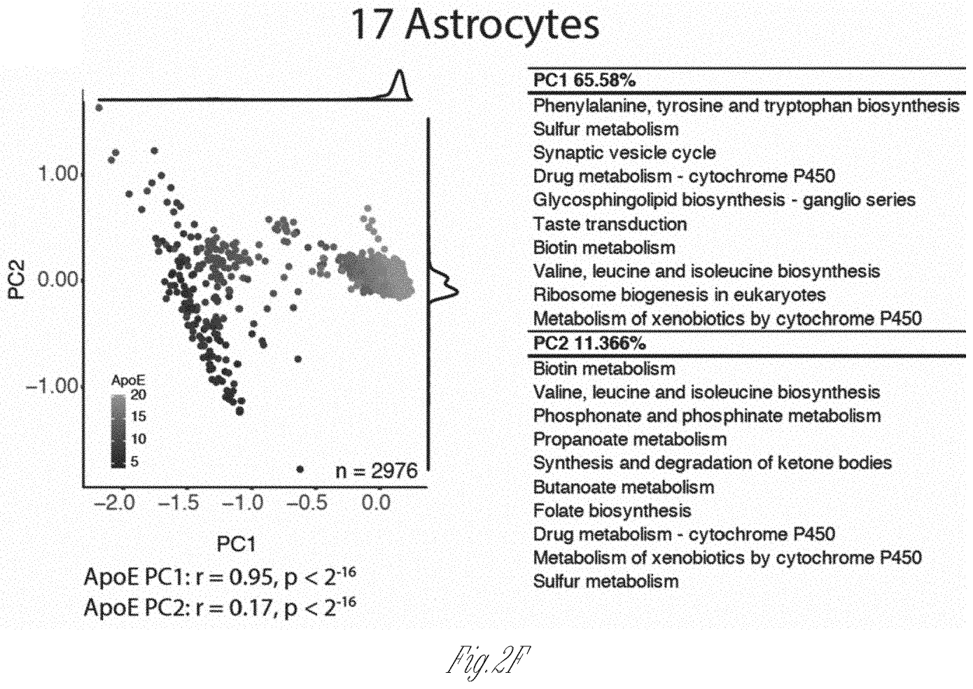

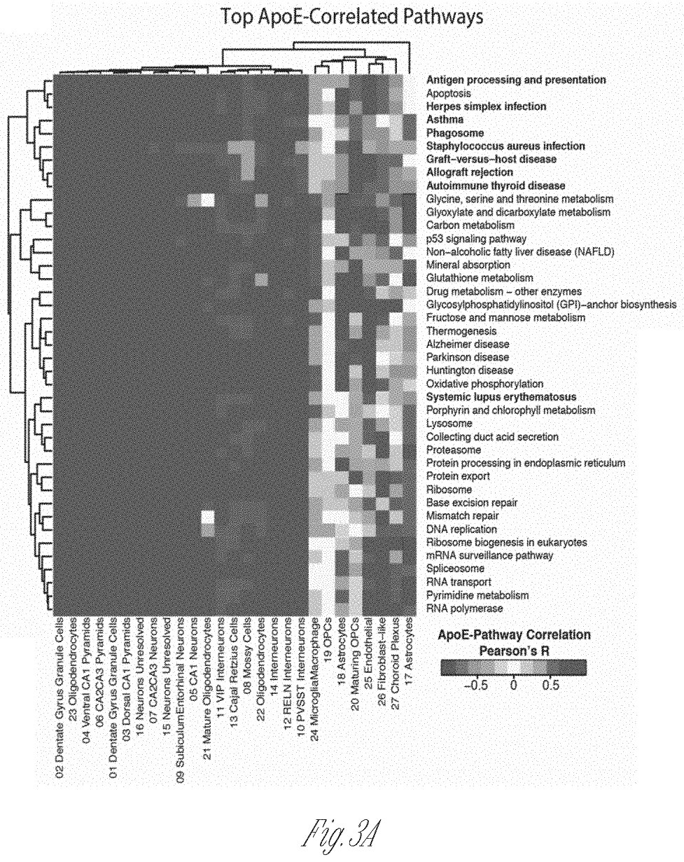

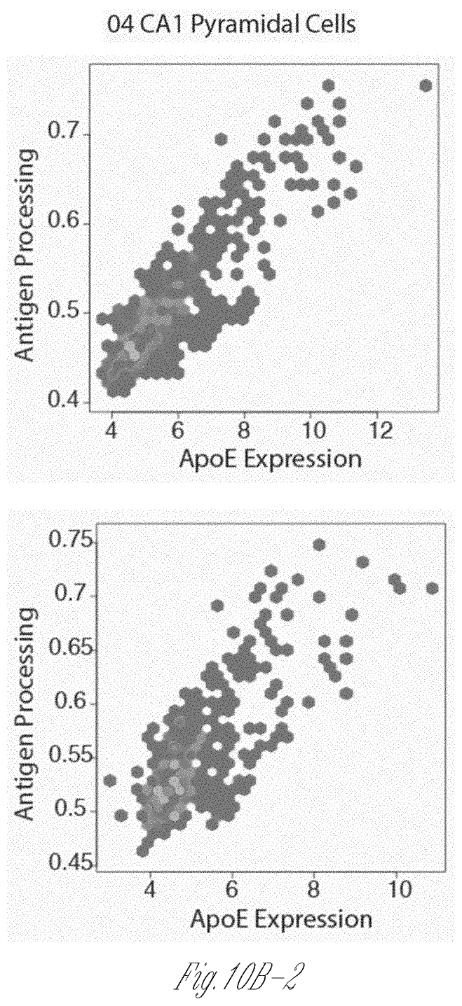

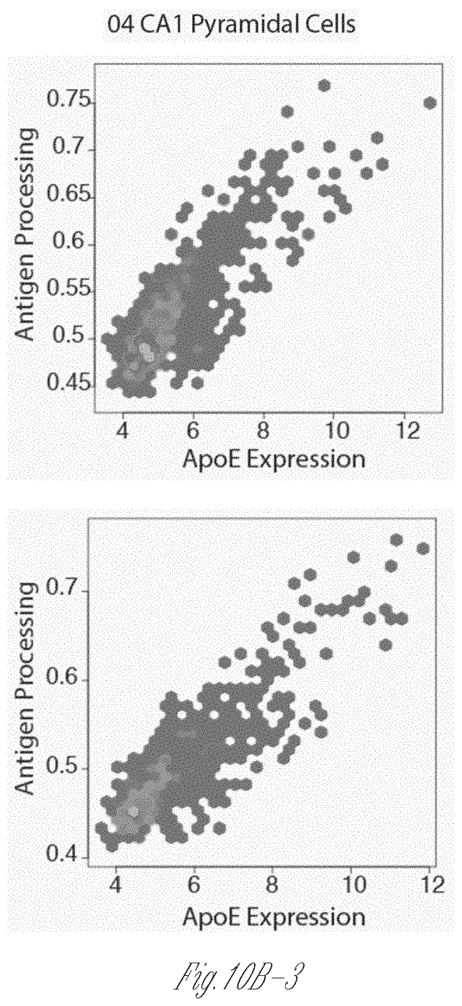

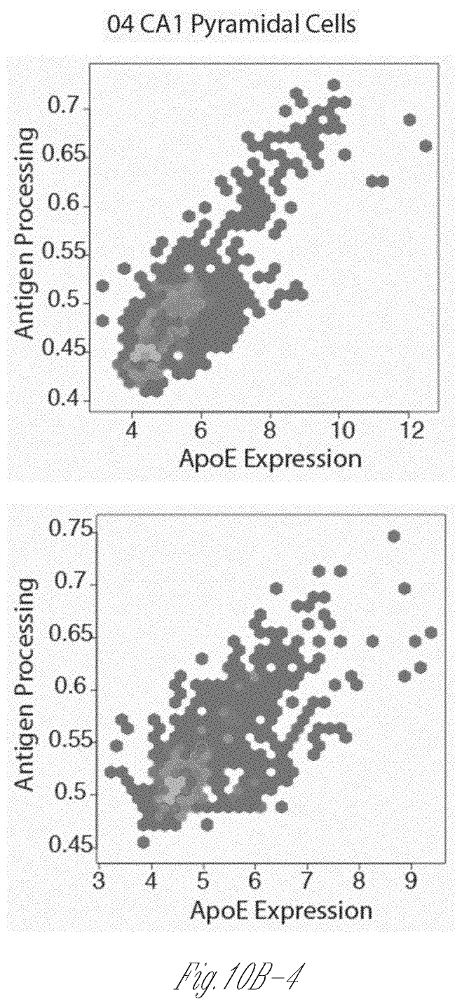

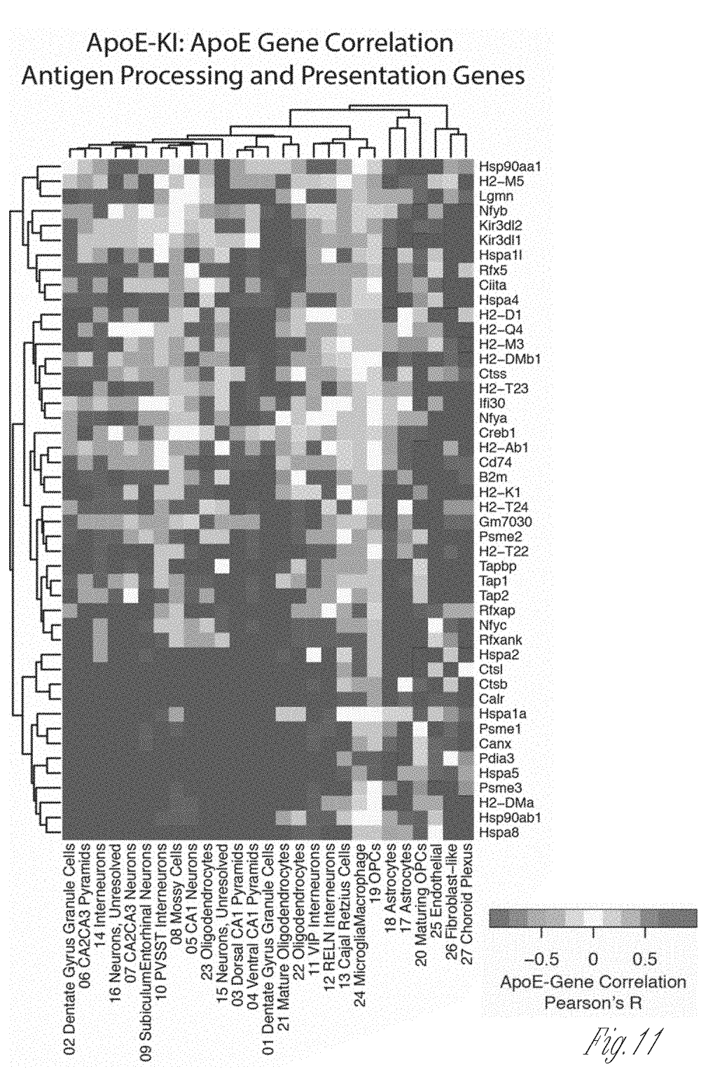

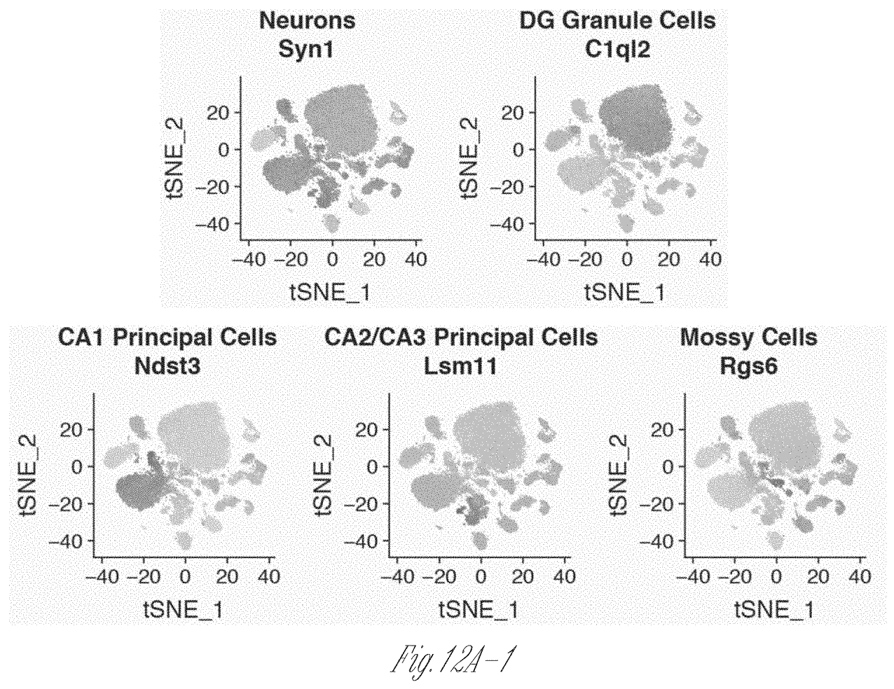

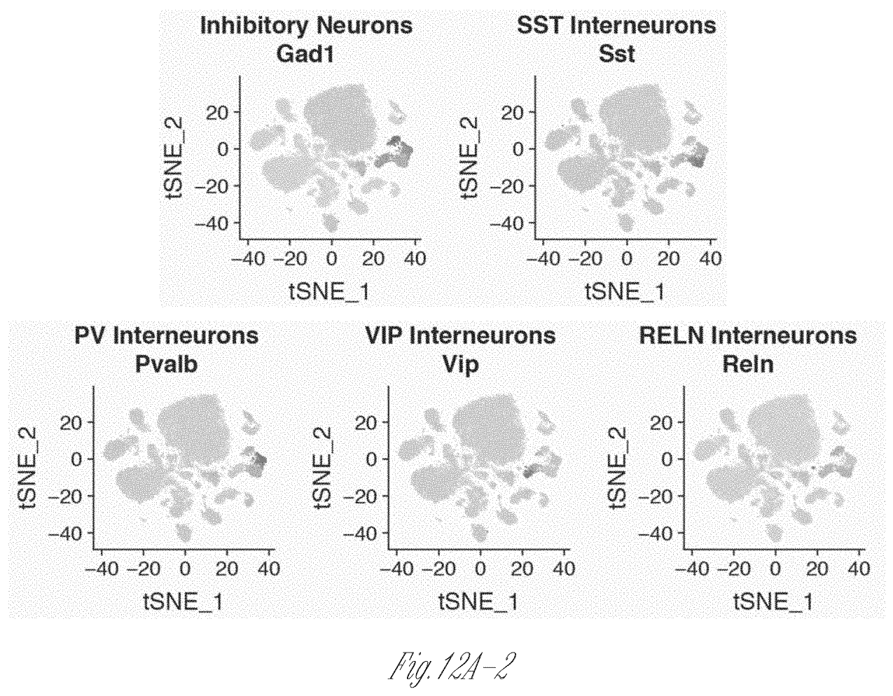

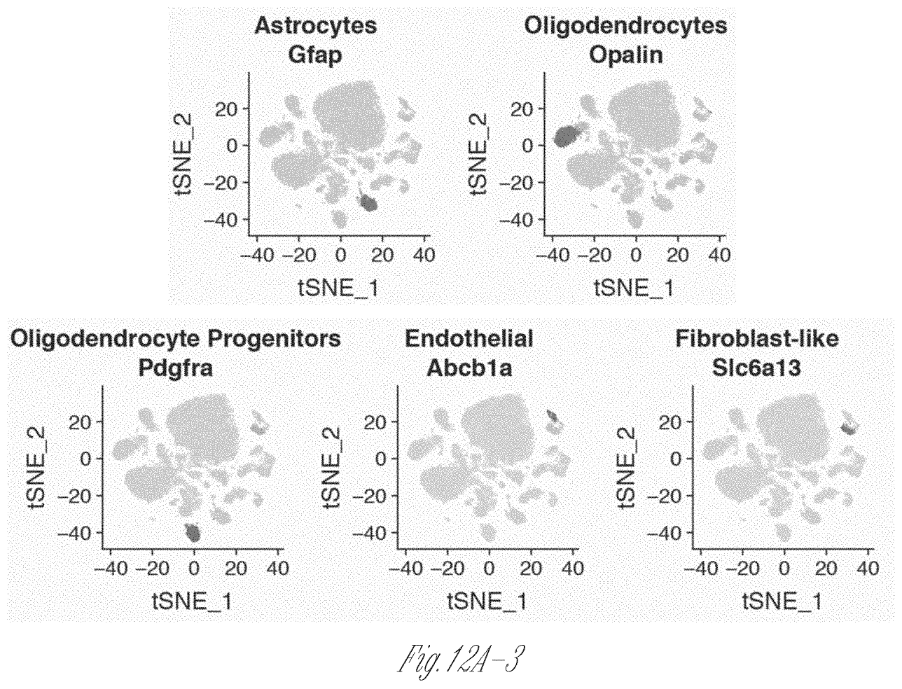

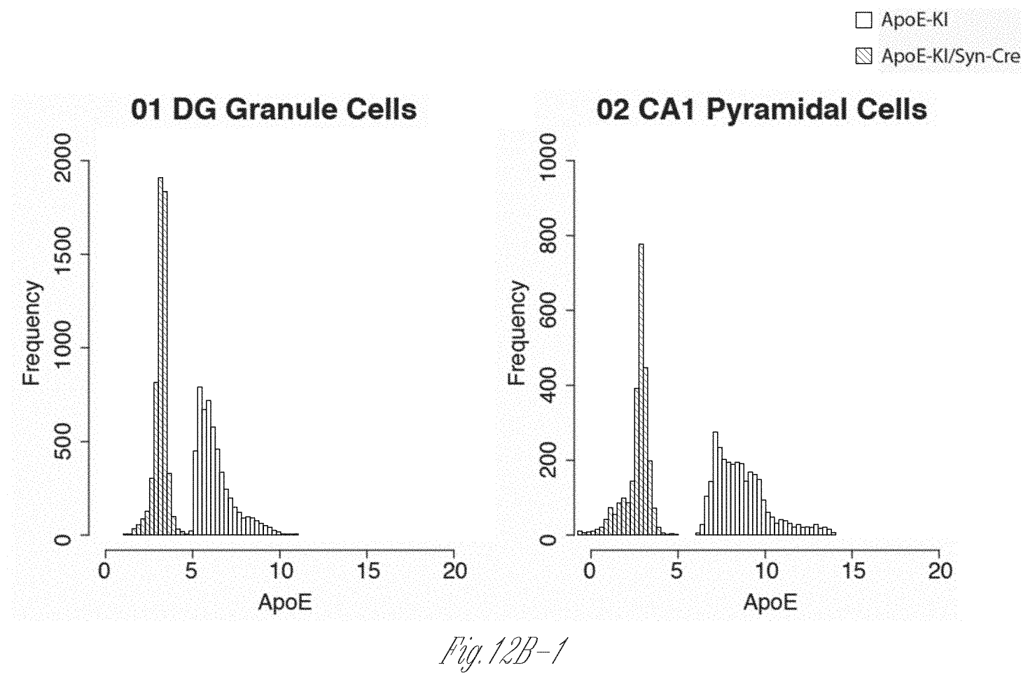

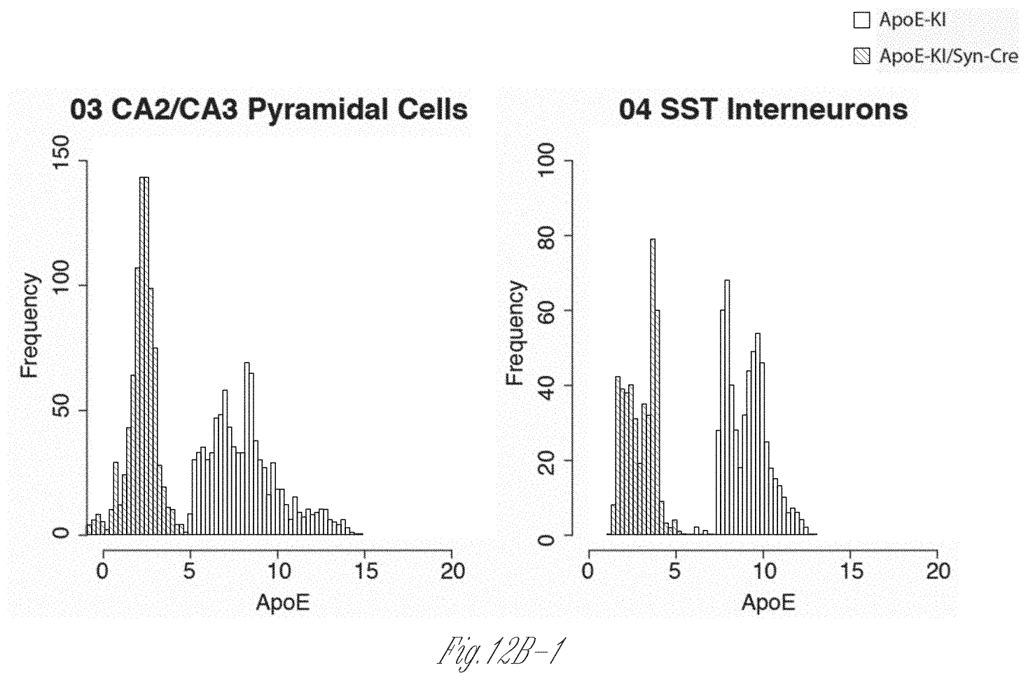

Single-nucleus RNA sequencing data was first generated on isolated hippocampi from female human apoE3-KI and apoE4-KI mice at 5, 10, 15, and 20 months of age (n=4 per genotype and age) (FIG. 1A). After normalization and filtering for quality control (see Method Details), the dataset contained 21,204 genes across 123,489 nuclei (FIG. 1B). Clustering by shared nearest neighbor (SNN) and visualization by t-stochastic neighbor embedding (tSNE) revealed 27 distinct cell-type clusters (Butler et al., 2018; Stuart et al., 2019). These clusters were matched to known cell types by examining expression of canonical cell-type specific genes (Zhang et al., 2014), expression of genes identified in publicly available mouse hippocampal single-cell RNA sequencing datasets (Cembrowski et al., 2016; Saunders et al., 2018), and the expression of each cluster's marker genes (see Method Details) in a publicly available resource of brain-wide in-situ hybridization images (Lein et al., 2007) (FIG. 8). These analyses together identified 16 distinct neuronal clusters, encompassing 105,644 cells, and 11 non-neuronal clusters, encompassing 17,845 cells (FIG. 1C). The nucleus isolation protocol that was used did not capture enough microglia for reliable RNA sequencing analysis.

Principal Component Analysis (PCA) Reveals Expression of apoE and Immune-Response Pathways as Major Contributing Factors to Cell-by-Cell Variance for Most Hippocampal Neuronal, but not Astrocytic, Cell Types in apoE-KI Mice

It was noted that, across neuronal types, apoE was expressed at variable levels, with a relatively high level in 7-10% of neurons. To further examine the implications of neuronal apoE expression, the data was processed using a Markov affinity-based graph method designed to uncover gene-gene interactions in single-cell RNA sequencing data (Dijk et al., 2018). To examine the relationship between apoE and other cellular processes on a cell-by-cell basis, each cell was further assigned an expression score for the genes in each Kyoto Encyclopedia of Genes and Genomes (KEGG) pathway (see Method Details) (Kanehisa and Goto, 2000). Each cell population was then visualized using PCA to obtain a view of the drivers of variability across individual cells of the same type.

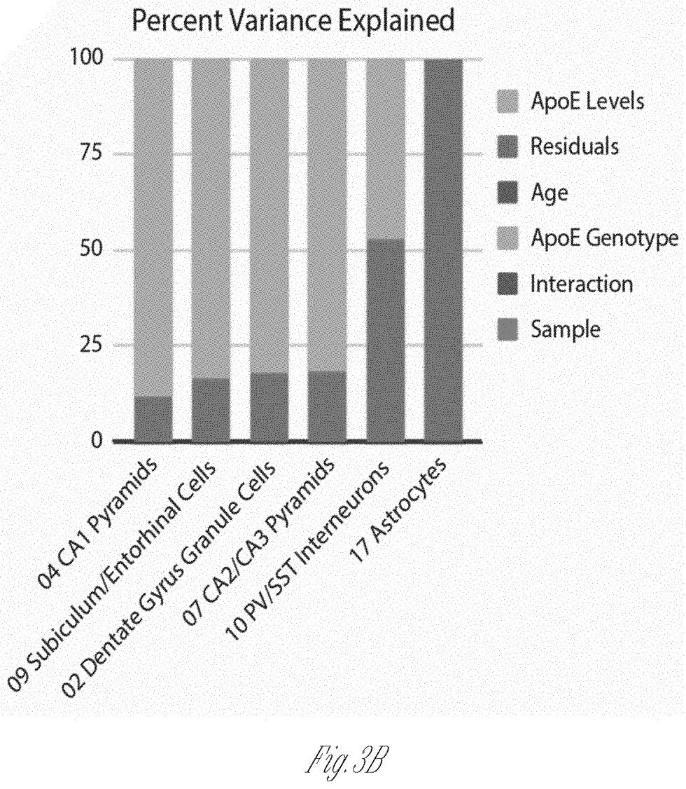

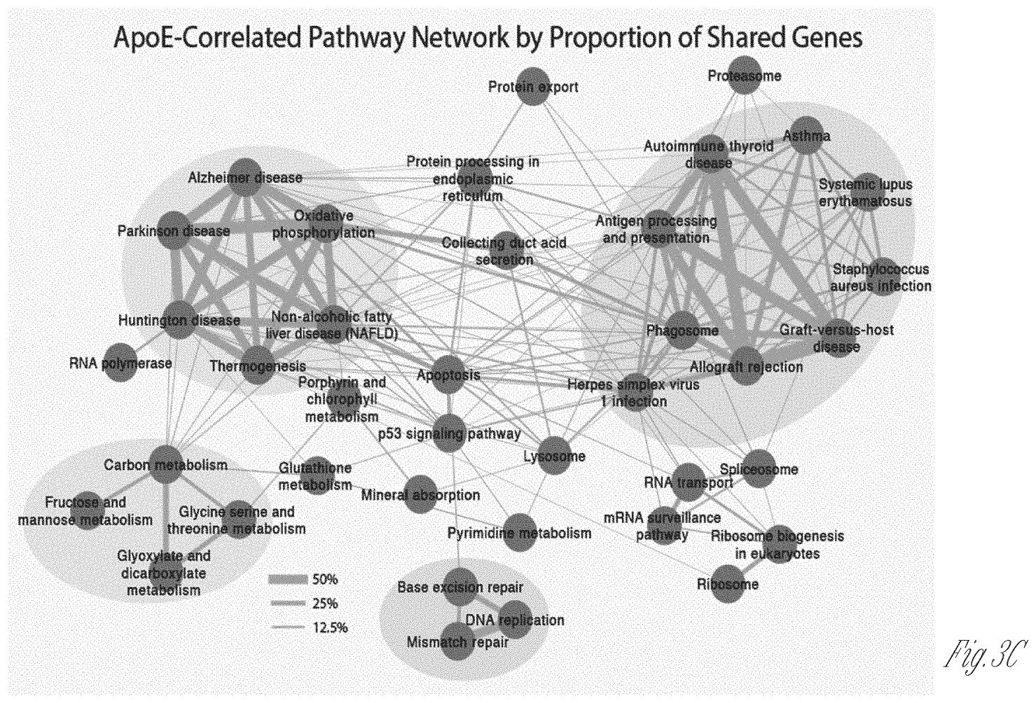

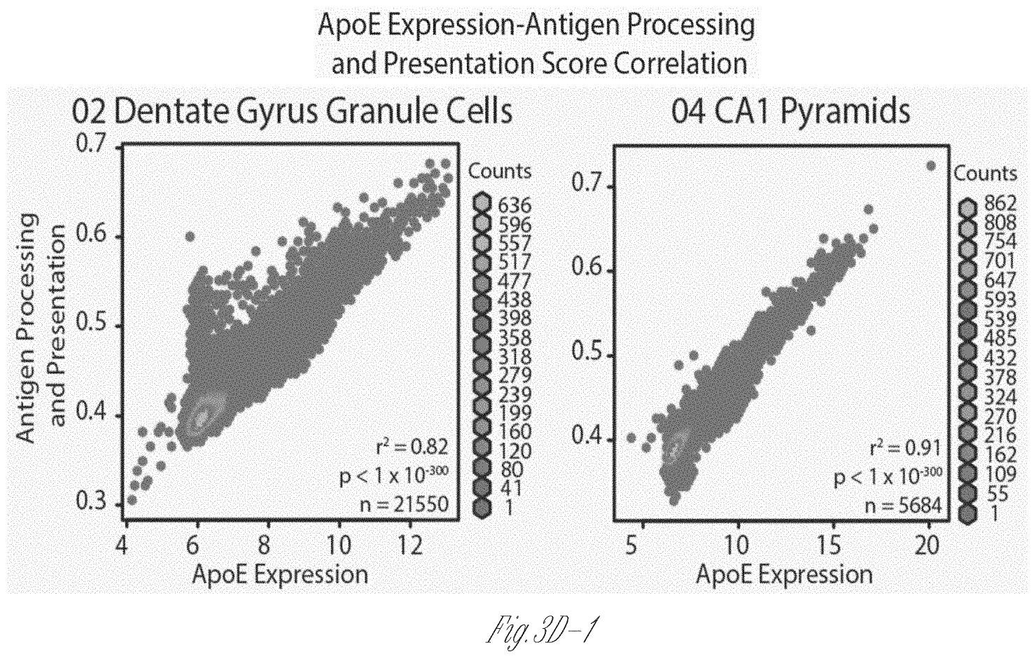

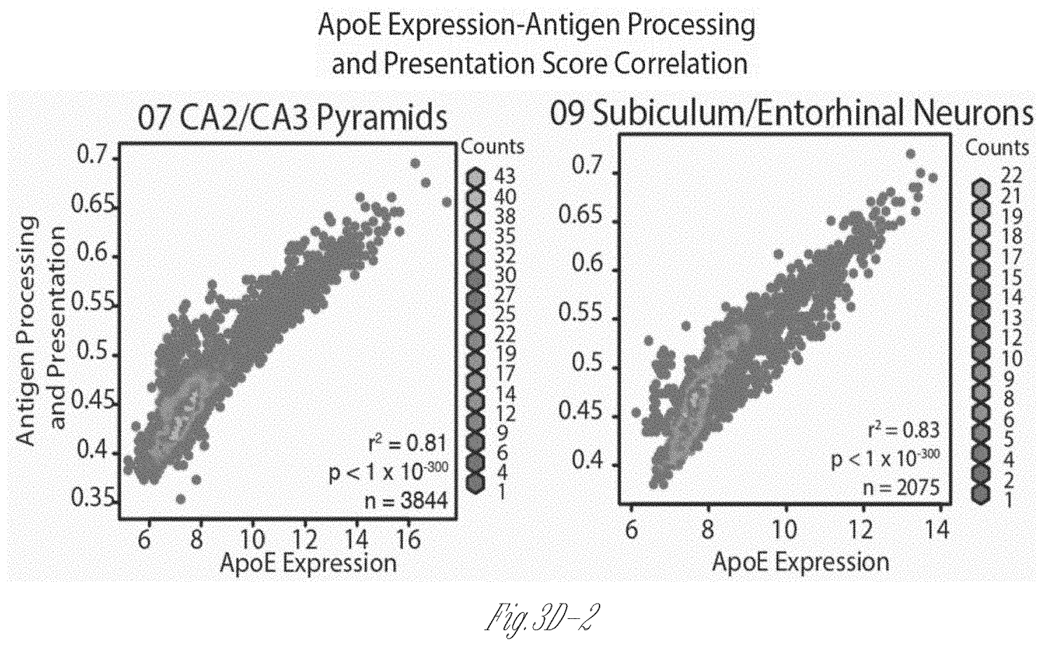

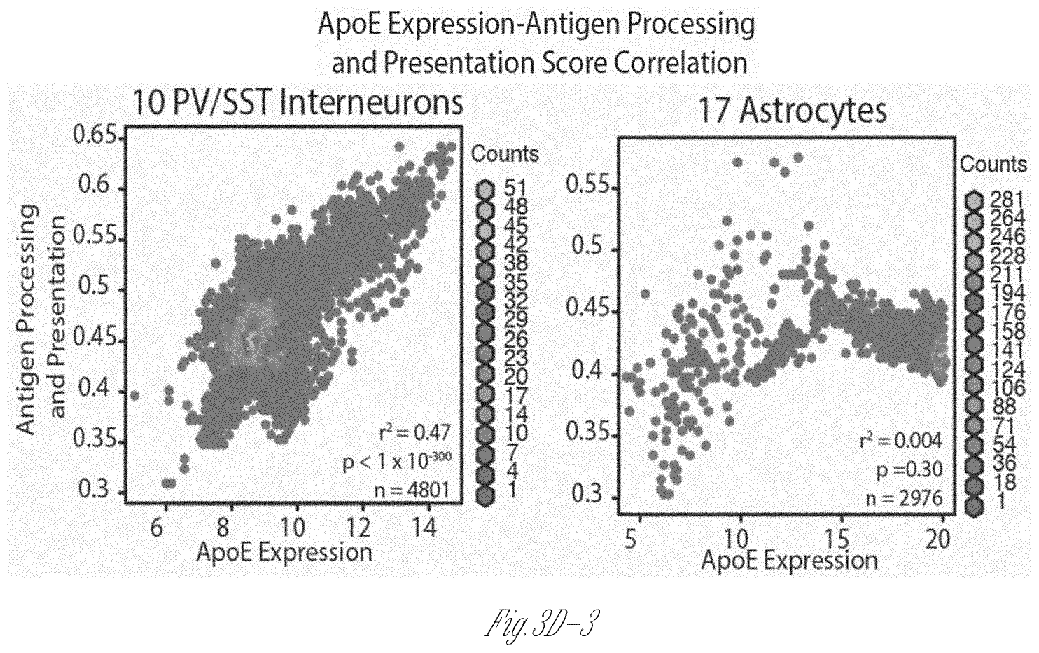

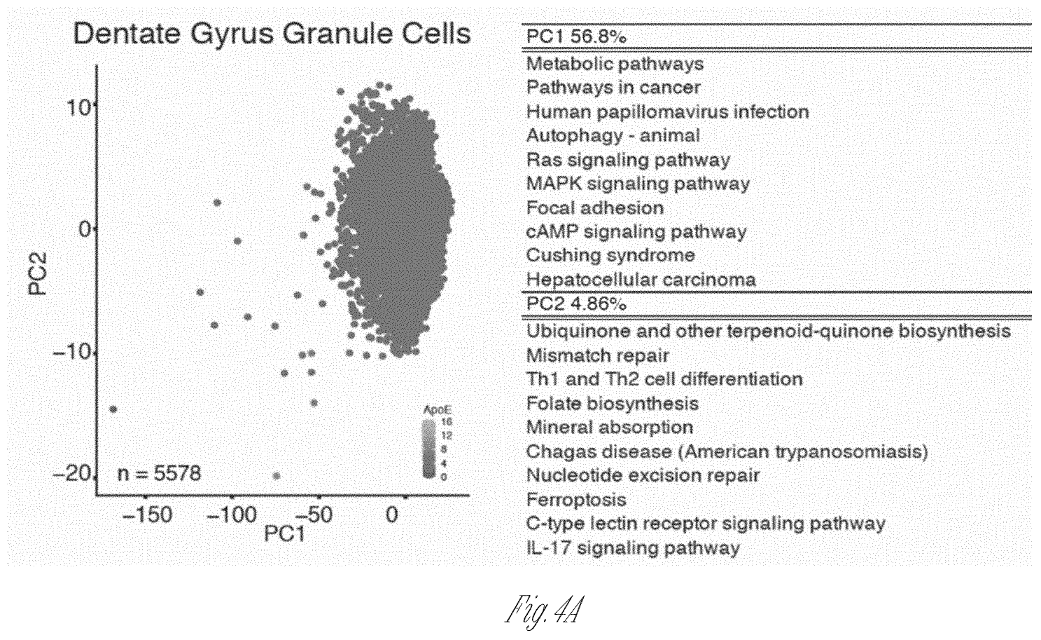

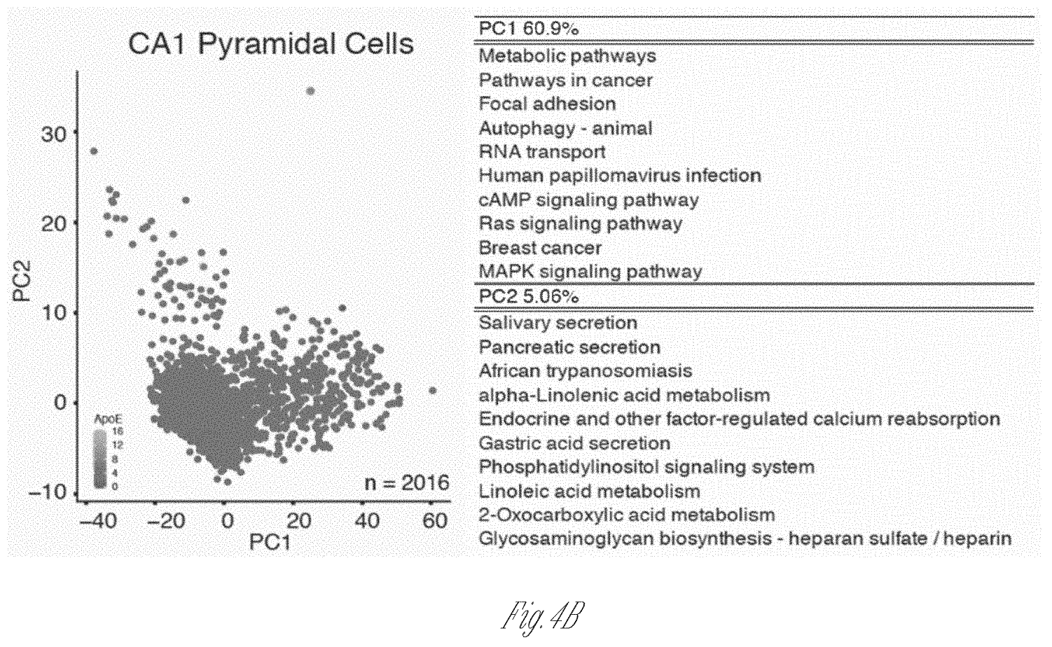

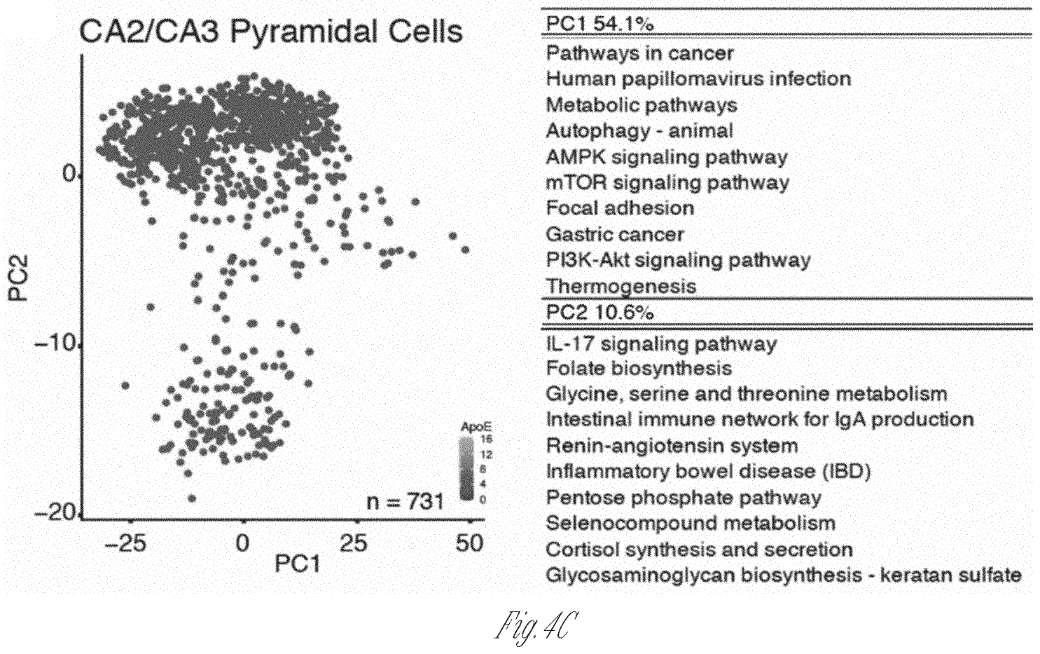

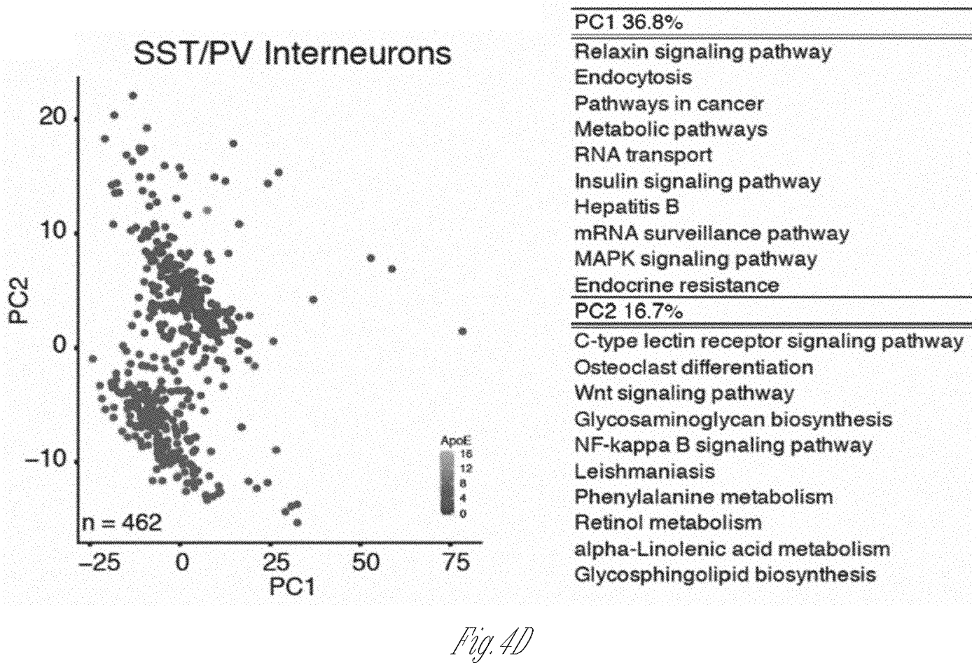

This analysis revealed two patterns across multiple neuron types: within-cell-type neuronal variability was largely driven by expression of MHC-related immune-response pathways, and these first PCs were highly correlated with apoE expression. In dentate gyms granule cells (FIG. 2A), for example, 6 of the top 10 pathway loadings for PC1 were immune-related. More specifically, 5 of the top 10 pathways involved expression of MHC genes (enrichment p<0.001), and this first PC, explaining 73% of within cell type variability, was directly correlated to levels of neuronal apoE expression (r=0.81, p<0.001). Similar levels of enrichment for MHC pathways and significant correlation between apoE and the first two PCs were seen across multiple other hippocampal neuron types, including CA1 pyramidal cells, CA2/CA3 pyramidal cells, subiculum/entorhinal cells, and SST/PV interneurons (FIG. 2B-2E). It was noted that these patterns are not driven by measures of read depth or quality, such as number of genes, number of unique molecular identifiers (nUMI), or percent mitochondrial genes (FIG. 9A-9D). Likewise, they were not explained by age or apoE genotype (FIG. 9E-9H), nor was there evidence of doublets, such as a decrease in neuron markers or an increase in astrocyte markers that tracked the gradient of apoE expression in these cells (FIG. 9I-9L).

In contrast, in astrocytes, although overall levels of apoE expression were much higher, and apoE expression correlated with the first principal component in these cells, this PC was defined by differences in expression of metabolic and biosynthesis pathways, rather than by expression of immune-related pathways (FIG. 2F). These findings together suggest that, uniquely in neurons, the primary axis of within-cell-type variability is defined by MHC signaling pathways, and that this variability is highly correlated with differences in neuronal apoE expression.

ApoE Expression Correlates with MHC Pathways in Most Hippocampal Neuronal, but not Astrocytic, Cell Types in apoE-KI Mice

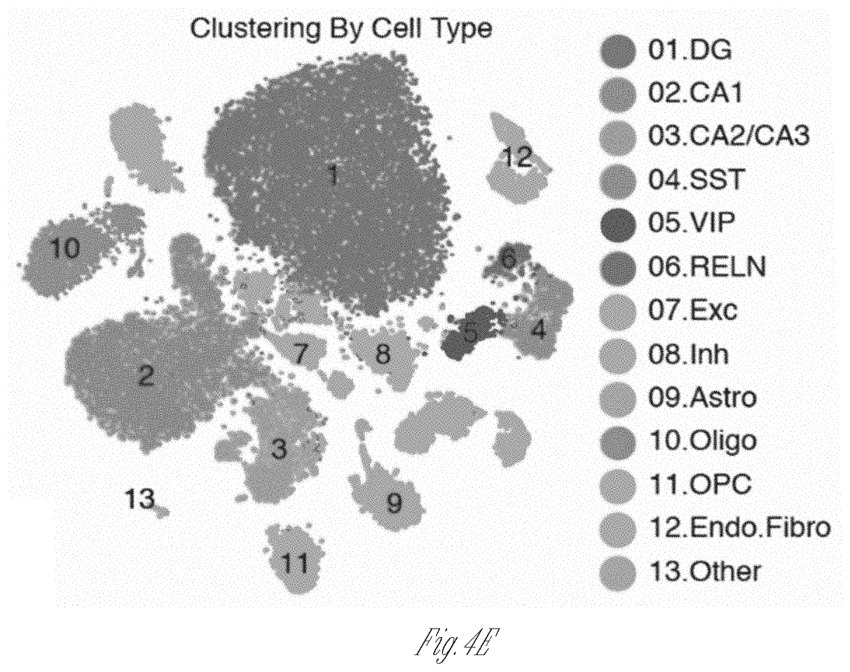

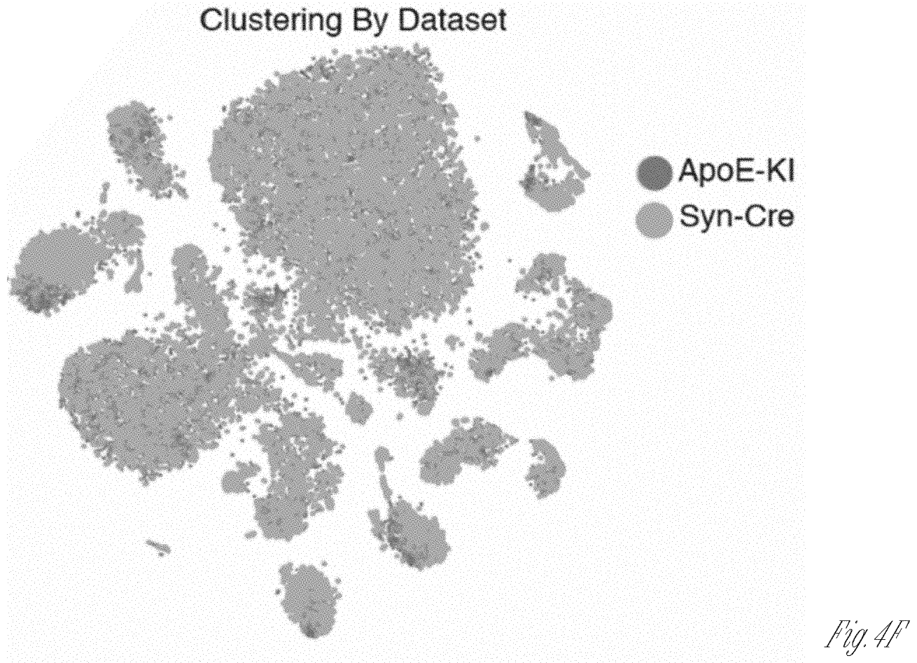

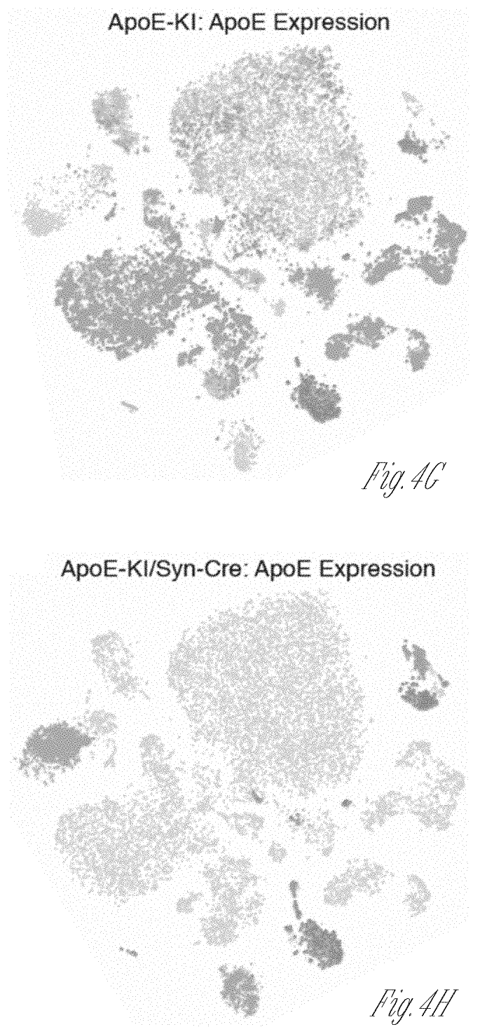

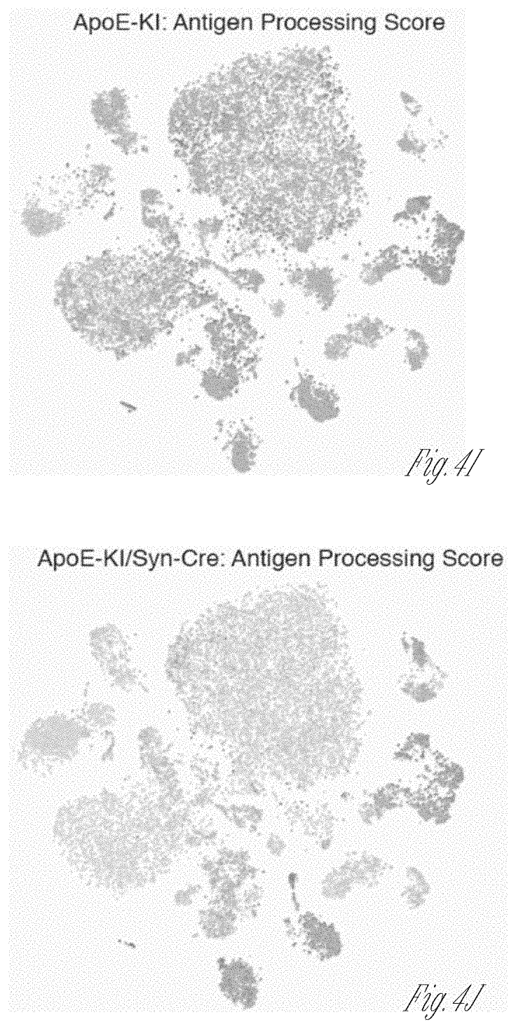

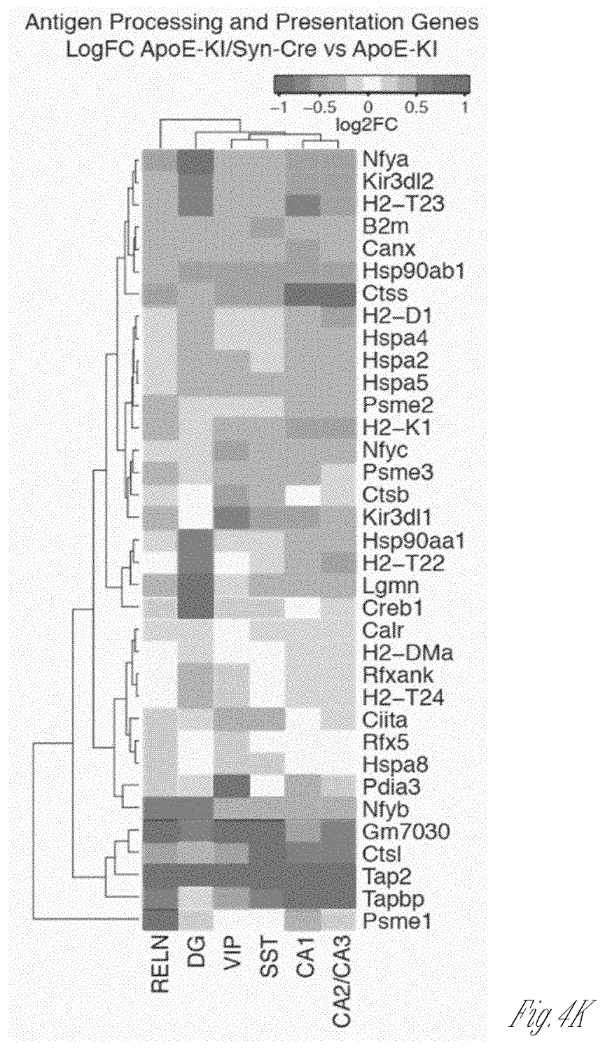

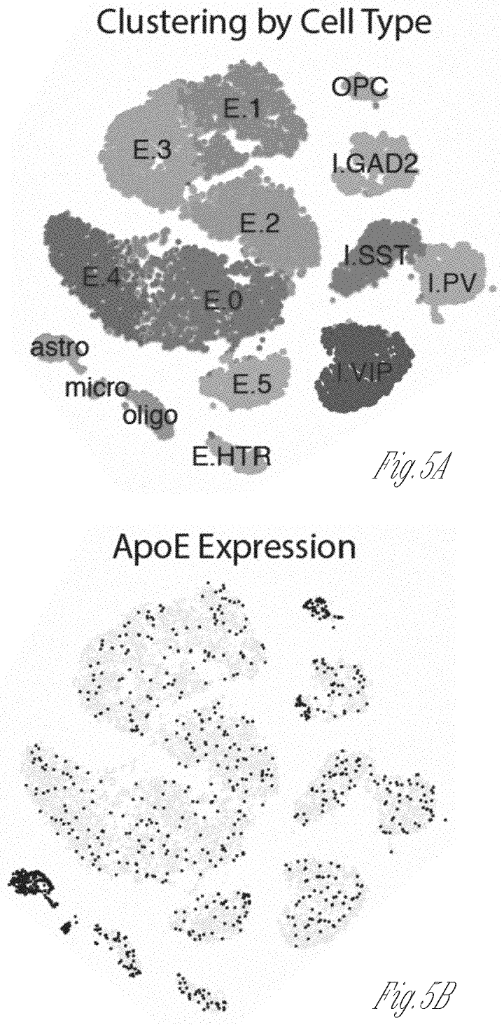

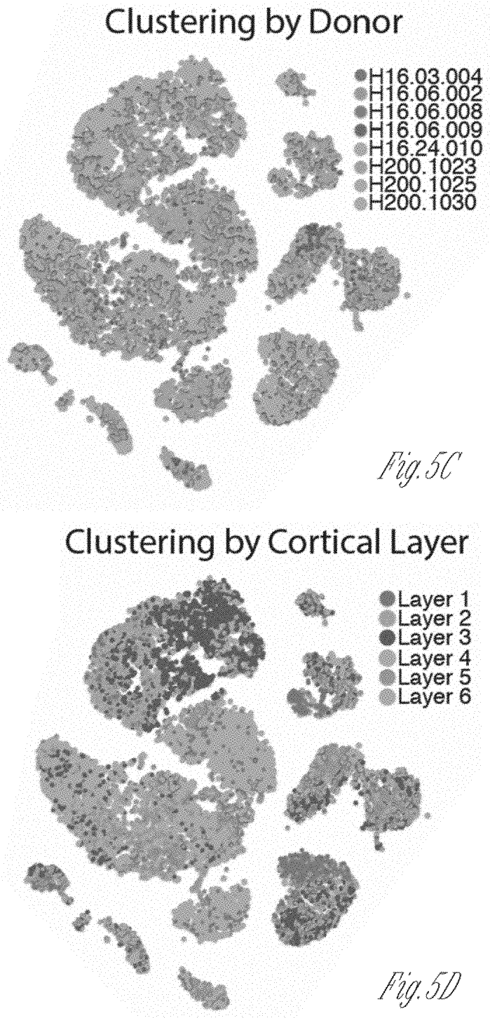

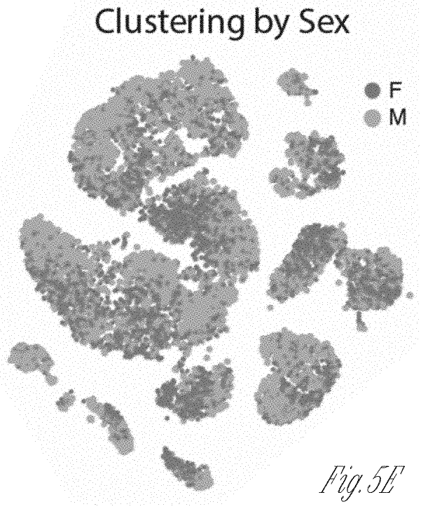

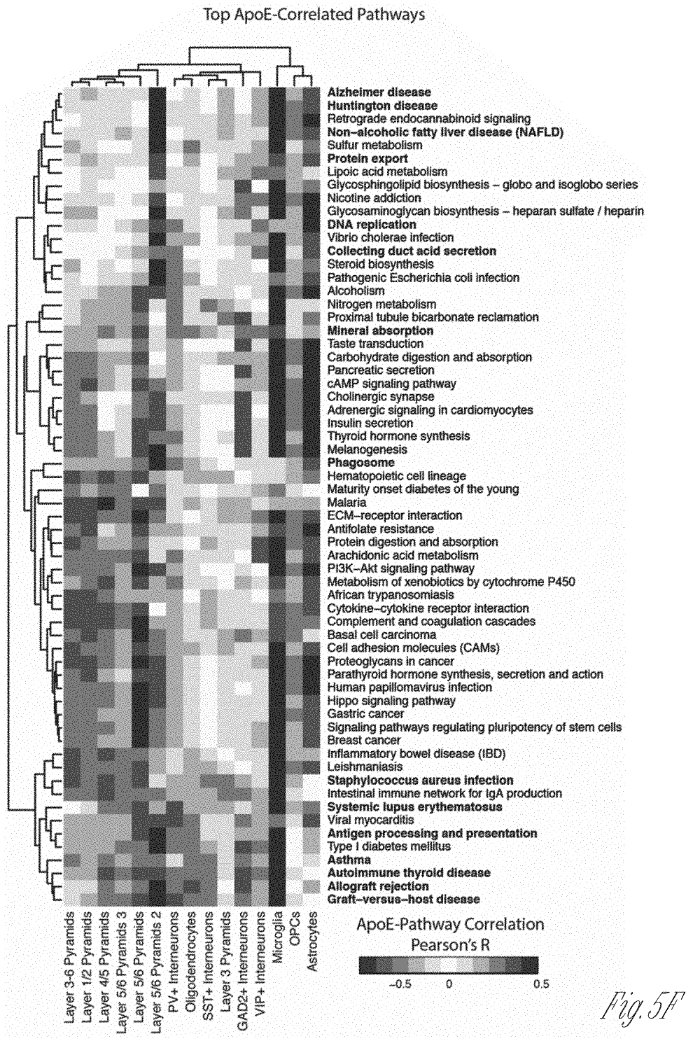

The unbiased search for drivers of within-cell-type variability suggested a relationship between neuronal apoE expression and neuronal MHC signaling (FIG. 2). To examine this possibility more directly, the KEGG pathways whose expression scores most correlate with apoE expression on a cell-by-cell basis within each neuron type were searched for. Hierarchical clustering of these apoE-by-pathway correlations was performed and the results were visualized as a heatmap (FIG. 3A). The relationship between apoE gene expression and the pathway scores was highly consistent, with minimal contribution from age or apoE genotype (FIG. 3B and FIG. 10), so all samples were combined for this analysis. Although this analysis could have generated a list of up to 160 pathways (10 unique pathways for each of the 16 neuronal types), the complete list was comprised of just 41 pathways, indicating a high degree of overlap across neuronal cell types. Additionally, 9 of the 41 pathways (22%; enrichment p=0.0002) were immune-related and contained MHC genes (highlighted in bold in FIG. 3A). Other pathways of particular interest included those related to cellular stress, such as apoptosis, proteasome, and p53 signaling, as well as those related to neurodegeneration, such as Alzheimer disease, Huntington disease, and Parkinson disease (FIG. 3A). Finally, an overall pattern of apoE-by-pathway correlations was noted, where these relationships were strongest in excitatory principal cells, of intermediate strength across a variety of inhibitory interneurons, and weakest and even anti-correlated in astrocytes, OPCs, endothelial, and choroid plexus cells, again suggesting cell-type-specific effects of apoE expression on cell signaling pathways.

In examining the relationships among the top neuronal apoE-correlated pathways, it was found that some pathways were relatively isolated in terms of shared genes, while others were tightly interconnected into modules (FIG. 3C). In these modules, some pathways that are expected to relate to neuronal apoE expression were found. For example, a module of densely-interconnected pathways related to neurodegenerative disease, including the Alzheimer disease, Huntington disease, and Parkinson disease pathways was found (FIG. 3C, orange). Additionally, a module related to cellular metabolism was found (FIG. 3C, green), and one related to DNA replication and repair was found (FIG. 3C, blue). Strikingly, however, the largest module of apoE-correlated pathways contained those that relate to MHC signaling (FIG. 3C, pink).

In further analyzing this module, it was found that the hub, the pathway with the largest edge-sum, was the antigen processing and presentation pathway. Interestingly, among the pathways most correlated with neuronal apoE expression in hippocampus principal neuron clusters, the antigen processing and presentation pathway was one of the most frequent; it was represented in the top 10 most correlated pathways in 6 out of 7 principal neuron clusters. By comparison, the Alzheimer's disease pathway was represented in 5 neuronal cell clusters and the Huntington's disease pathway was represented in 4 neuronal cell clusters.

Because of its privileged place amongst neuronal apoE-correlated pathways, examining the antigen processing and presentation pathway and its cell-by-cell relationship with apoE expression was focused on. A strong and significant correlation was found between apoE expression and the antigen processing and presentation score across multiple neuronal types, including dentate gyms granule cells, CA1 principal cells, CA2/CA3 principal cells, subiculum/entorhinal cells, and SST/PV interneurons (FIG. 3D). The relationship between apoE expression and the expression of each individual gene in the antigen processing and presentation pathway followed a similar pattern to the pathway as a whole: strong positive correlations between apoE expression and the gene set in excitatory principal neurons, intermediate-strength correlation across inhibitory interneurons, and mixed, weak, or negative correlation in other cell types (FIG. 11). Strikingly, although astrocytes express much higher baseline levels of apoE, their apoE expression showed no significant relationship with antigen processing and presentation pathway (FIG. 3D).

Neuron-Specific Knockout of the APOE Gene Abolishes MHC Pathways as Major Contributing Factors to Cell-by-Cell Variance of Hippocampal Neurons in apoE-KI Mice

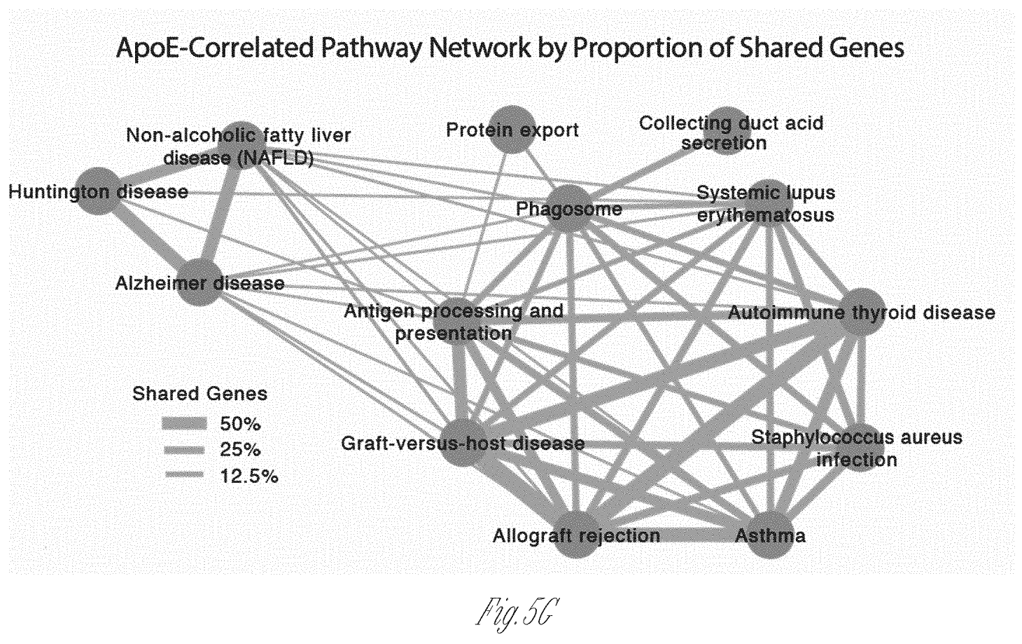

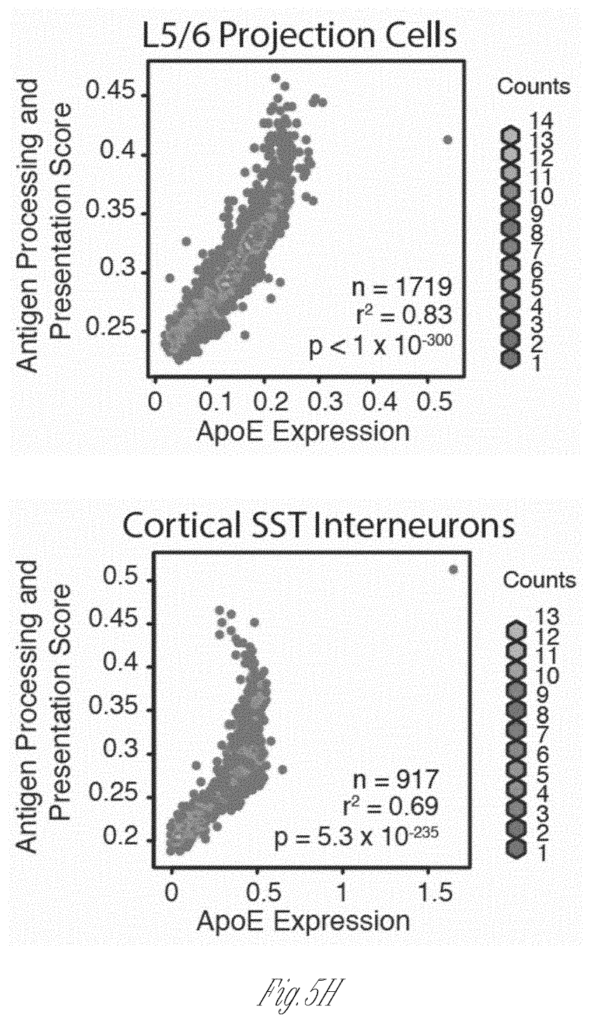

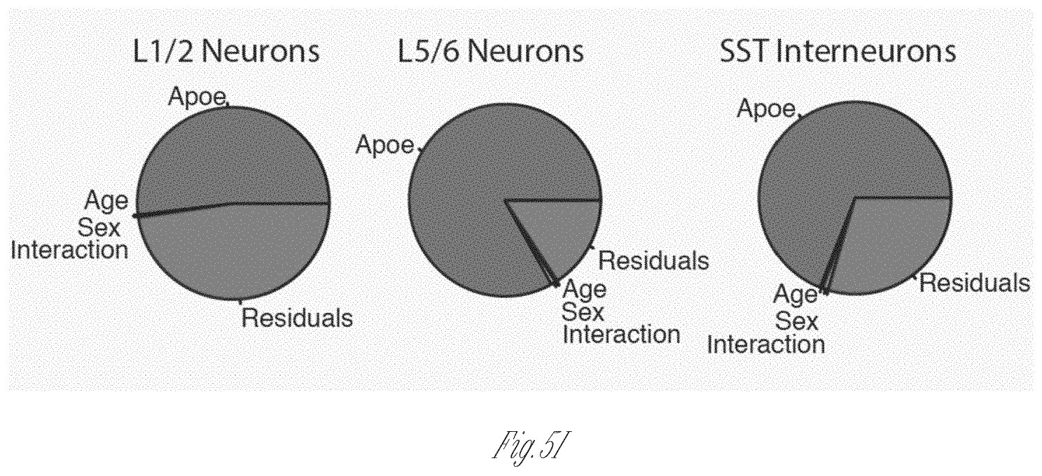

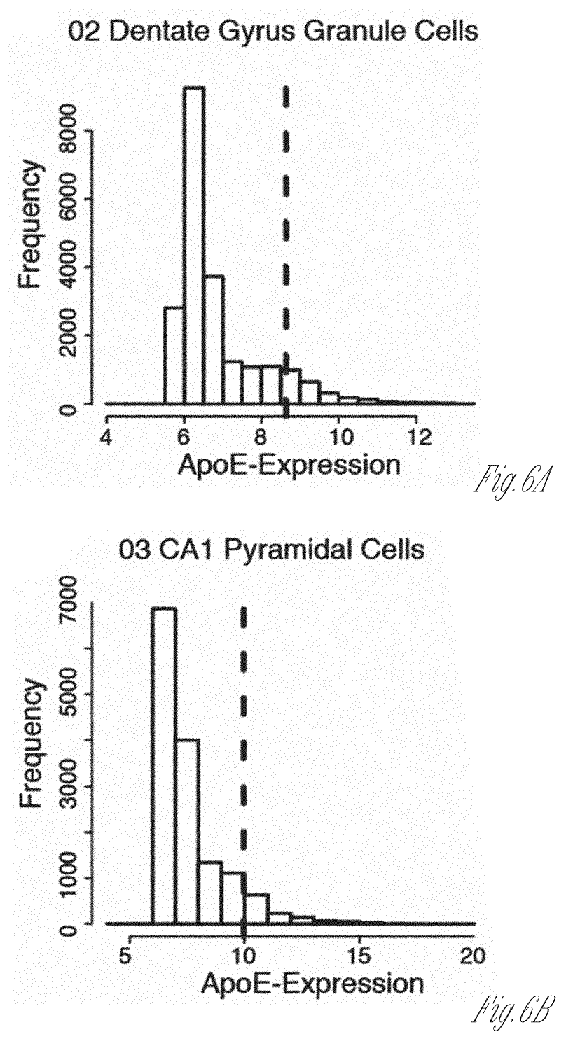

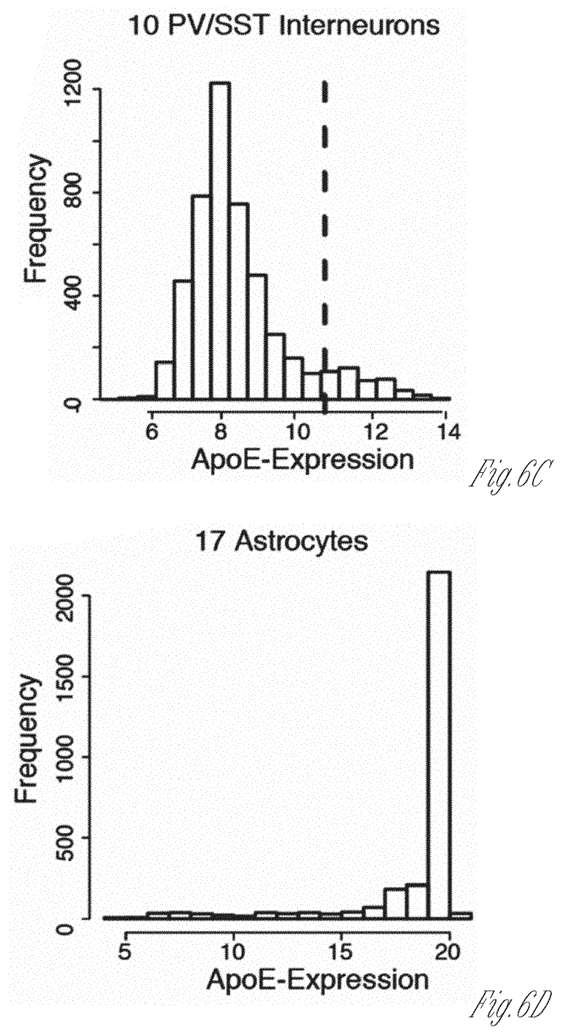

Although these MHC signaling pathways were strongly correlated with neuronal apoE expression, it was unclear to what degree neuronal apoE expression is necessary for driving these within-cell-type differences. To examine this question, single-nucleus RNA sequencing was performed on four aged (14-16 months) apoE-KI mice with the APOE gene specifically floxed out of neurons (apoE-KI/Syn-Cre mice). These mice express human apoE in all cells except neurons. The same analyses were then performed on these cells, as displayed in FIG. 2. Now, with the APOE gene knocked out of neurons, an enrichment of immune or MHC-signaling pathways was no longer found among the top pathways that define PC1 and PC2 of these cells (FIG. 4A-4D). Instead, these PCs were dominated by metabolic pathways, as well as intracellular and intercellular signaling pathways, such as Ras signaling, MAPK signaling, and cAMP signaling pathways. In addition, a number of cancer-related pathways, which are enriched for Wnt and Notch signaling components were seen.

To examine the differences between apoE-KI and apoE-KI/Syn-Cre neurons more directly, the data from the 15-month-old apoE-KI mice was combined together with the 15-month-old apoE-KI/Syn-Cre mice using canonical correlation analysis (see Method Details; FIG. 4E-4F). Representation in both datasets of all major cell types, including dentate gyms granule cells; CA1 and CA2/CA3 principal cells; SST, PV, RELN, and VIP interneurons, oligodendrocytes, OPCs, astrocytes, and endothelial/fibroblast cells was seen (FIGS. 4E and 12A). In addition, a marked reduction of apoE expression specific to neurons in apoE-KI/Syn-Cre mice was seen, as expected (FIGS. 4G and 4H). These data additionally indicate that even neurons expressing a low level of apoE are truly apoE-expressing, as the levels detected in apoE-KI neurons are markedly above noise levels detected in the apoE-KI/Syn-Cre neurons (FIG. 12B). It was noted that, in the apoE-KI neurons, but not other cell types, the expression of apoE closely tracks the expression of the antigen processing and presentation pathway on a cell-by-cell basis (FIGS. 4G and 4I). As predicted, in neurons of the apoE-KI/Syn-Cre mice, where the expression of apoE was specifically knocked out, the expression of the antigen processing and presentation pathway was also strongly reduced (compare FIGS. 4J to 4I). In examining expression of the individual genes that comprise the antigen processing and presentation pathway, significant reduction of multiple MHC-I genes across neuronal cell types was found, including H2-K1, H2-D1, H2-T22, and H2-T23 (FIG. 4K). A strong expression reduction in genes encoding proteins necessary for functional location of MHC to the cell surface, including Tapbp, Tap2, and B2m was also noted (FIG. 4K), together suggesting a reduction in the functional expression of MHC in neurons. These data strongly suggest that neuronal apoE expression truly drives differences in neuronal expression of MHC pathways, which are themselves the dominant component of within-cell-type variability amongst hippocampal neurons.

Neuronal apoE Expression Correlates with MHC Pathways in Human Brains

Having shown that neuronal apoE expression upregulates neuronal expression of MHC pathways in mouse brains, the next step was to examine whether the observed effects of neuronal apoE expression are relevant to human brains. To address this question, a dataset of single-nucleus transcriptomes from human temporal cortex was examined (FIG. 5A-5E). In this dataset, it was observed that a proportion of each neuronal type expressed apoE at a high level (FIG. 5B). This dataset was processed in a parallel manner to the mouse single-nucleus RNA sequencing, assigning a pathway expression score for every KEGG pathway to every cell (see Method Details), and queried the pathways whose expression most correlates with apoE expression within each neuron type (FIG. 5F). Of the 62 pathways identified, 15 were shared with the mouse data (24%, enrichment p=0.0035) and 8 contained MHC genes (13%; enrichment p=0.01).

In further characterizing the pathways that were shared between mouse and human datasets, it was found that they fell into two densely interconnected modules. As in the mouse, one module was related to neurodegenerative diseases, while the other related to MHC signaling (FIG. 5G). Again, as in the mouse, most neuronal subtypes demonstrated a strong correlation between apoE expression and the expression of the antigen processing and presentation pathway. This was true across both excitatory and inhibitory subtypes (FIG. 5H). Again, it was seen that a linear model describing the cell-by-cell relationship between apoE expression and the antigen processing and presentation pathway attributed a large amount of the variance to APOE gene expression, with negligible contributions from subjects' age, APOE genotype, and sex (FIG. 5I).

ApoE4-KI Mice Exhibit Increased Proportions of apoE-Expression-High Neuronal Cells at an Earlier Age than do apoE3-KI Mice

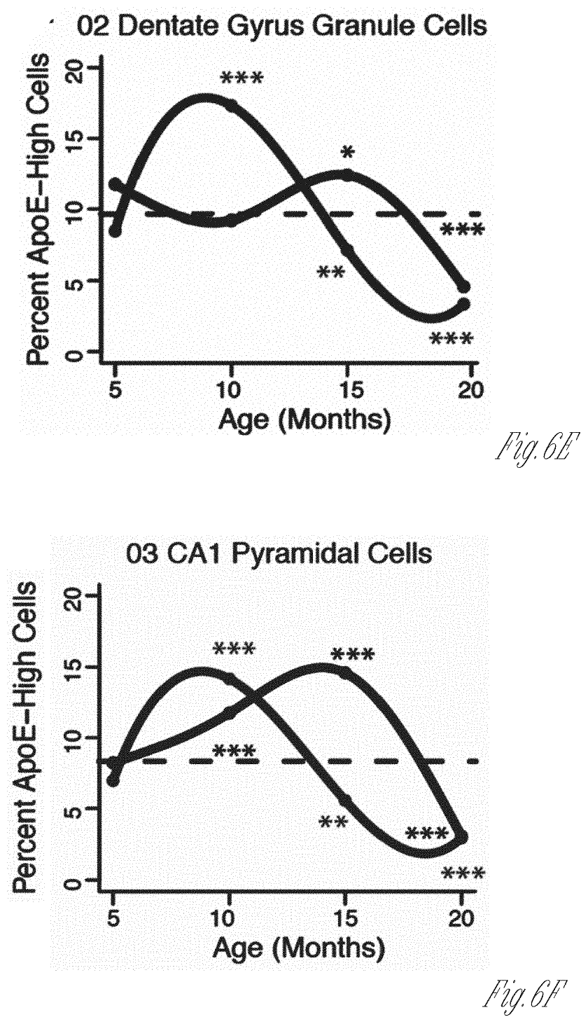

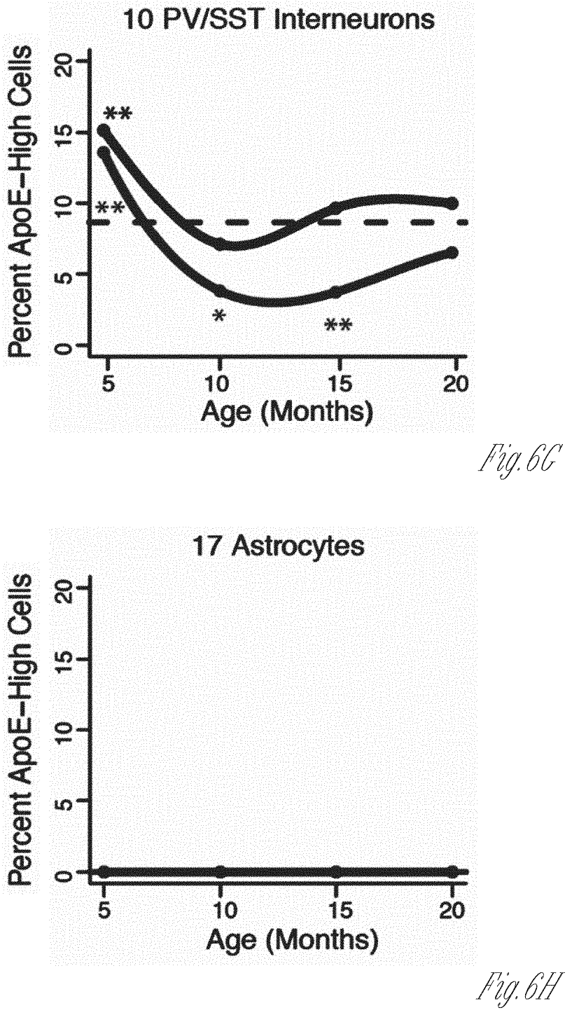

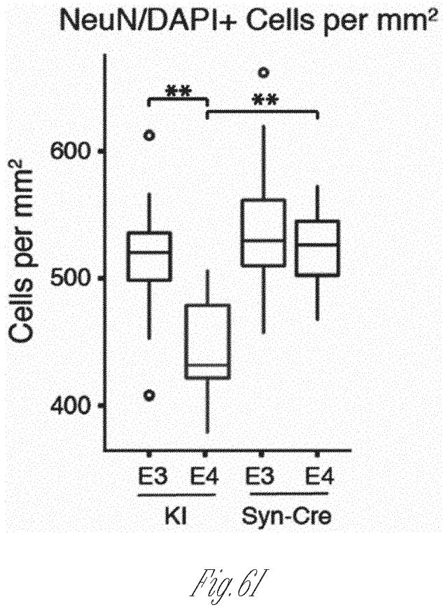

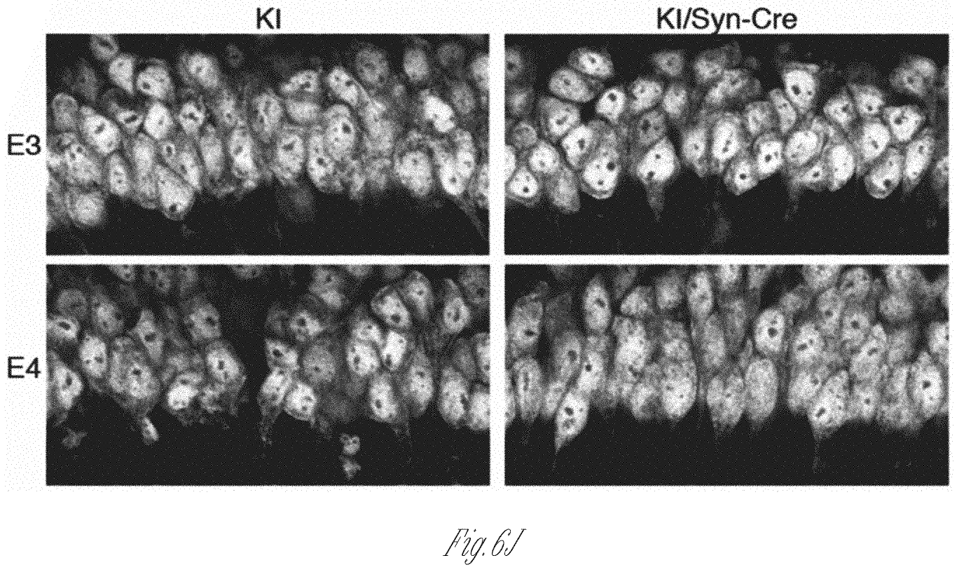

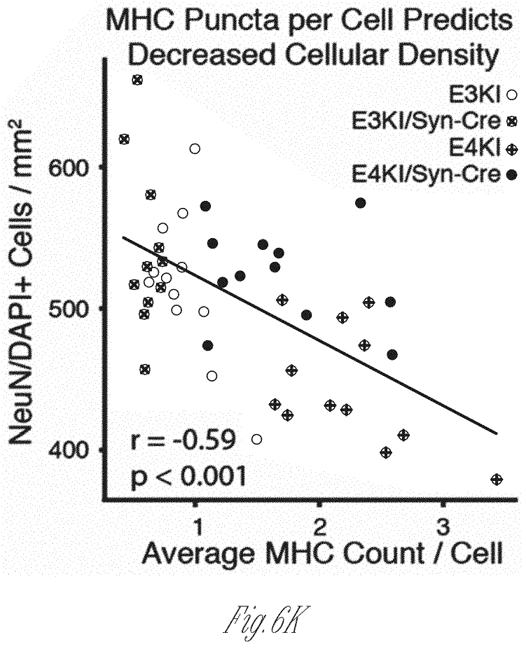

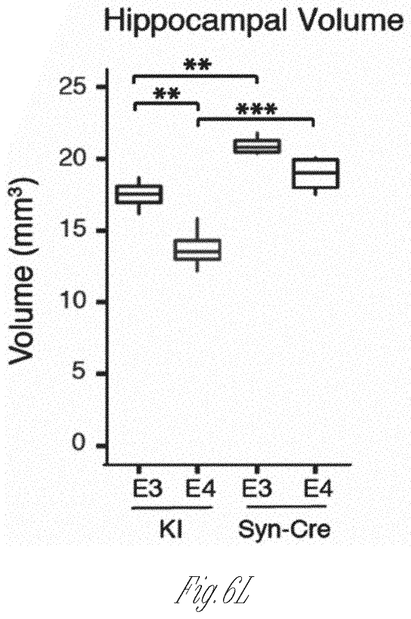

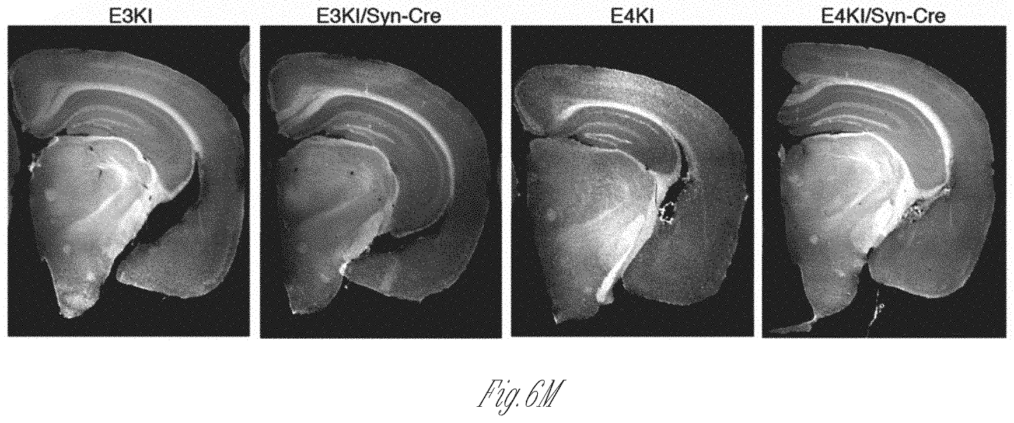

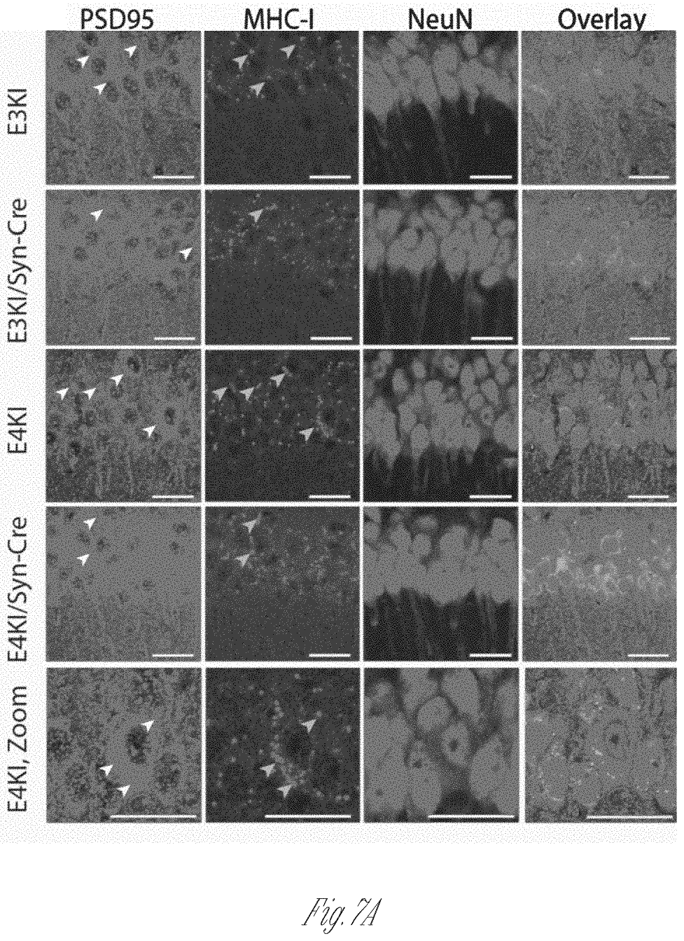

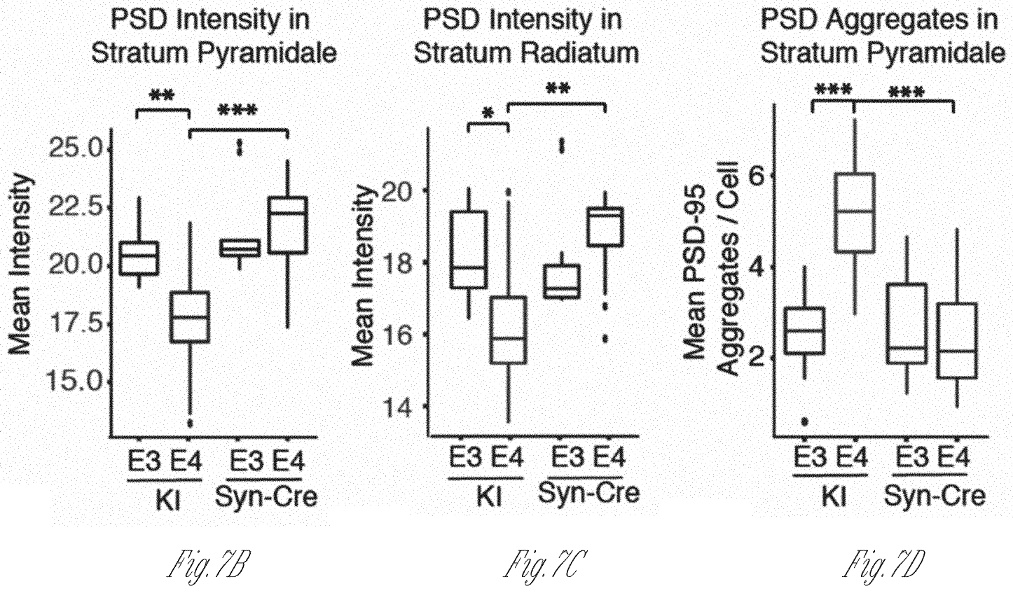

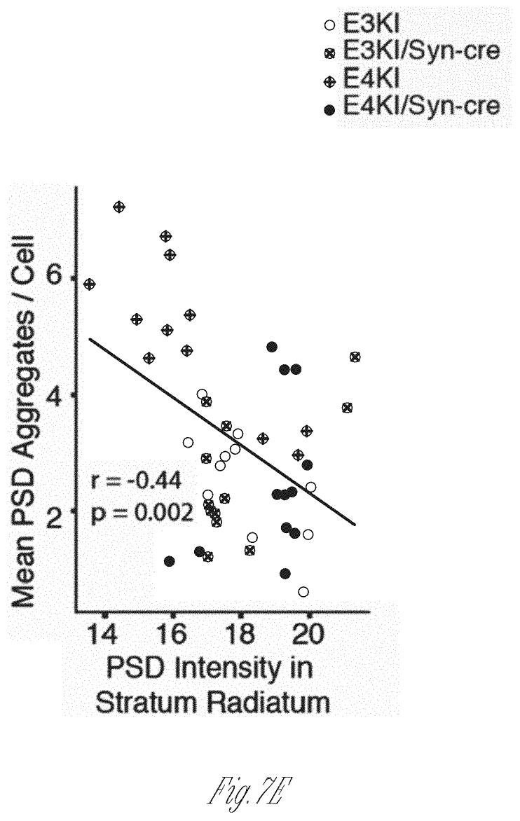

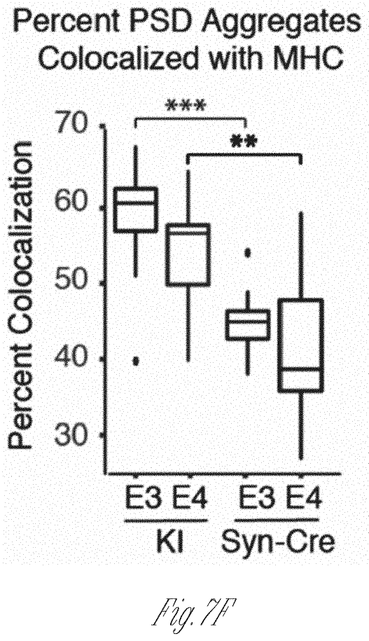

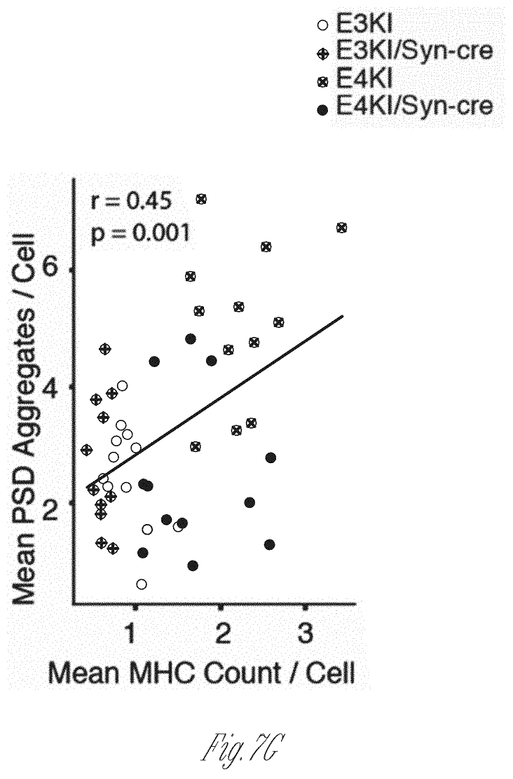

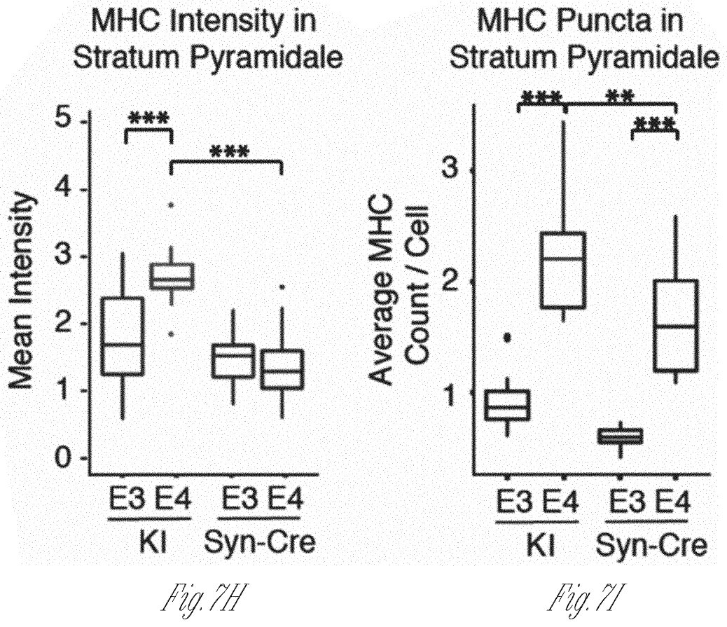

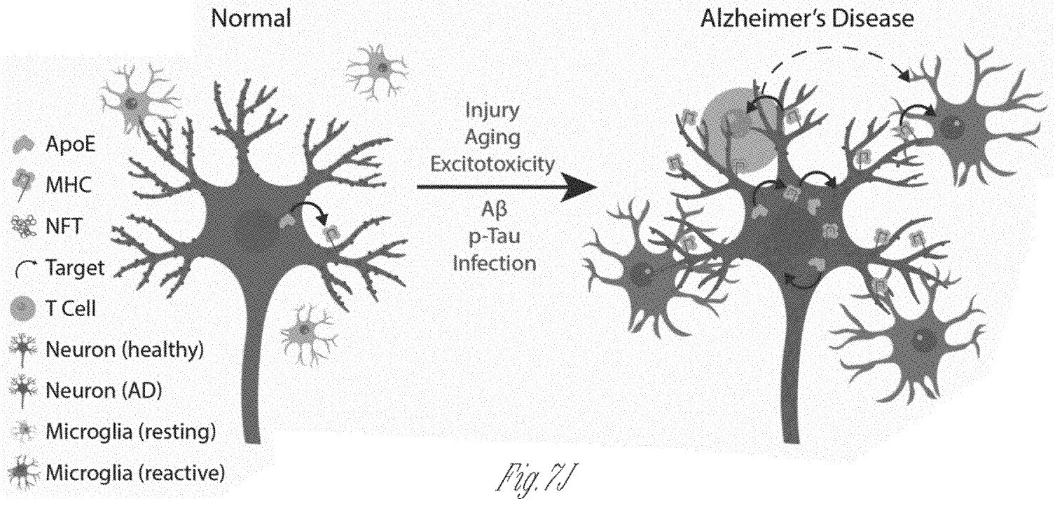

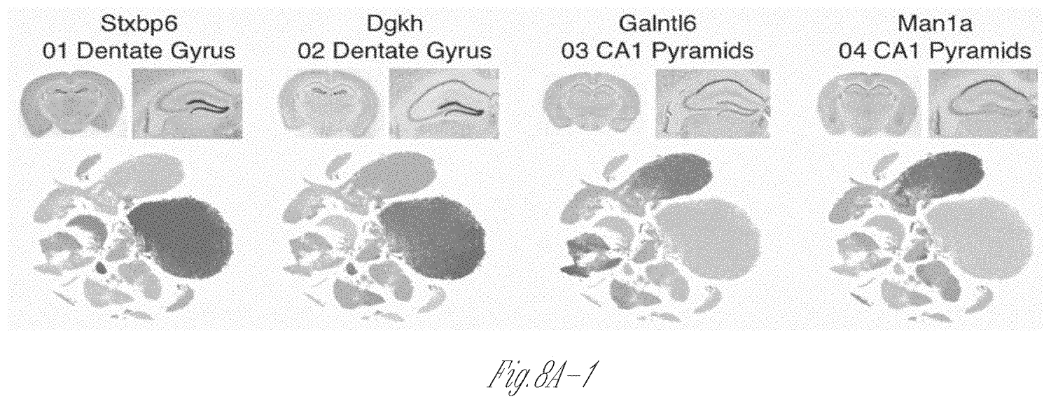

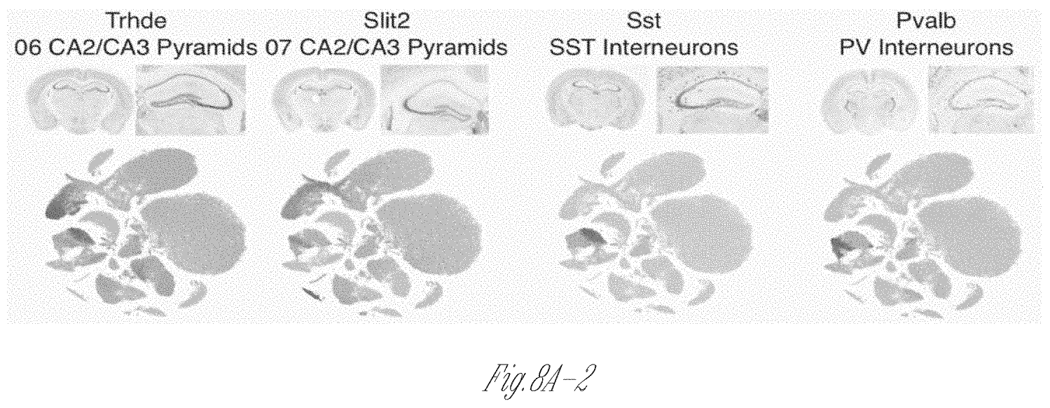

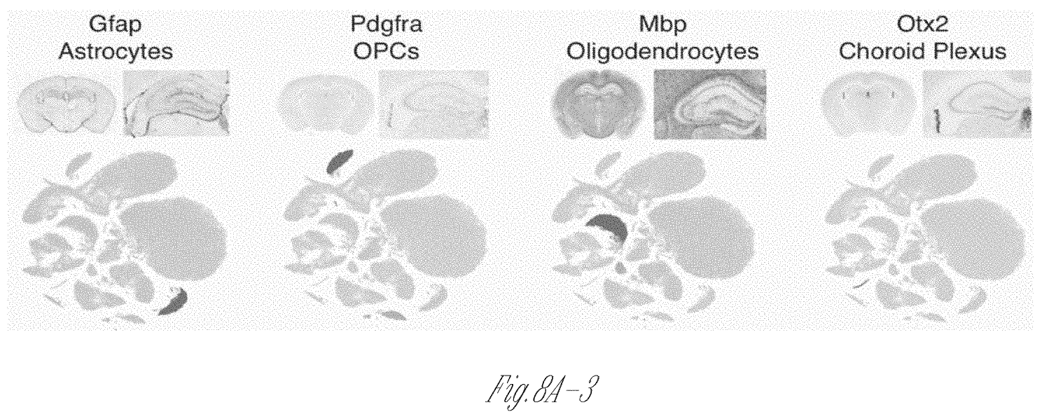

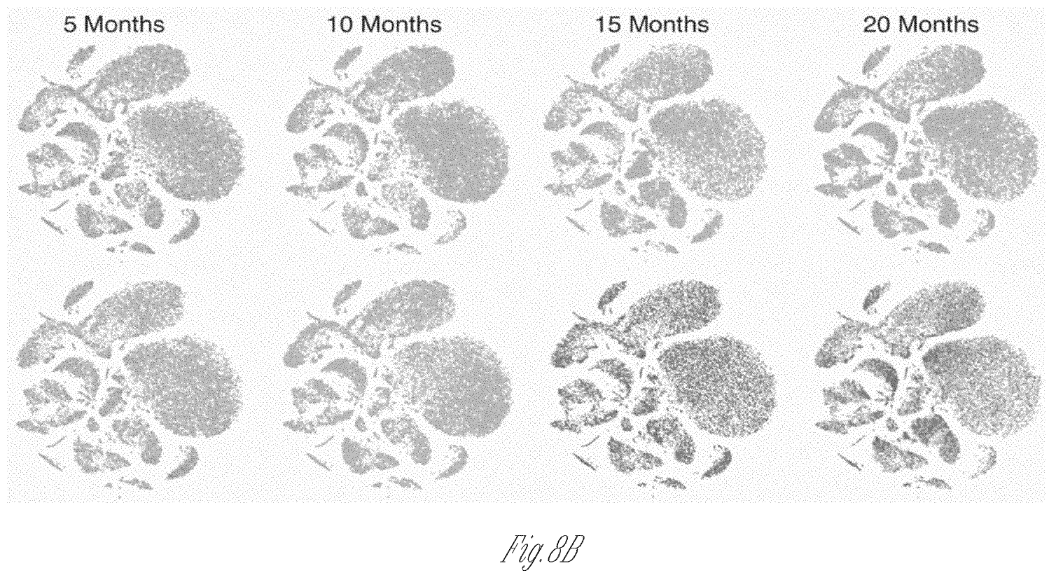

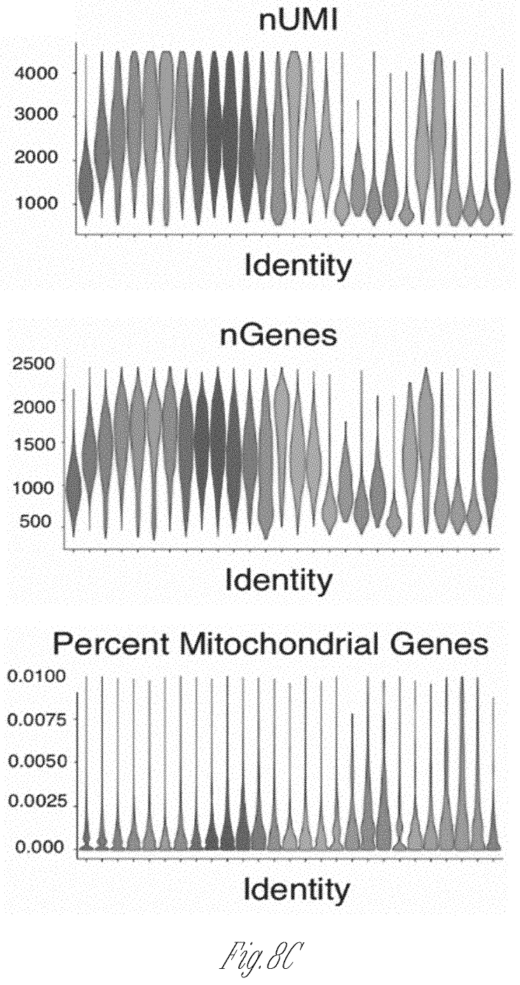

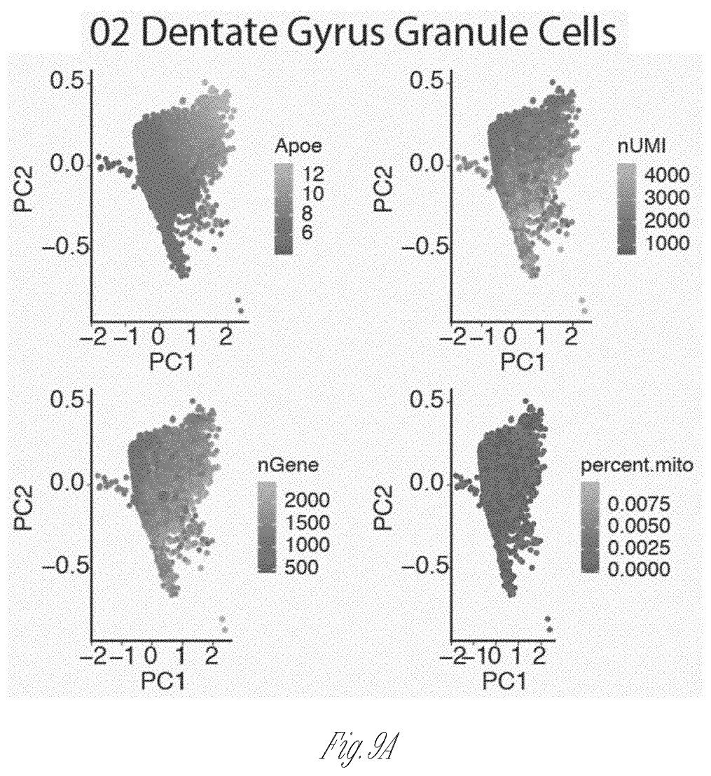

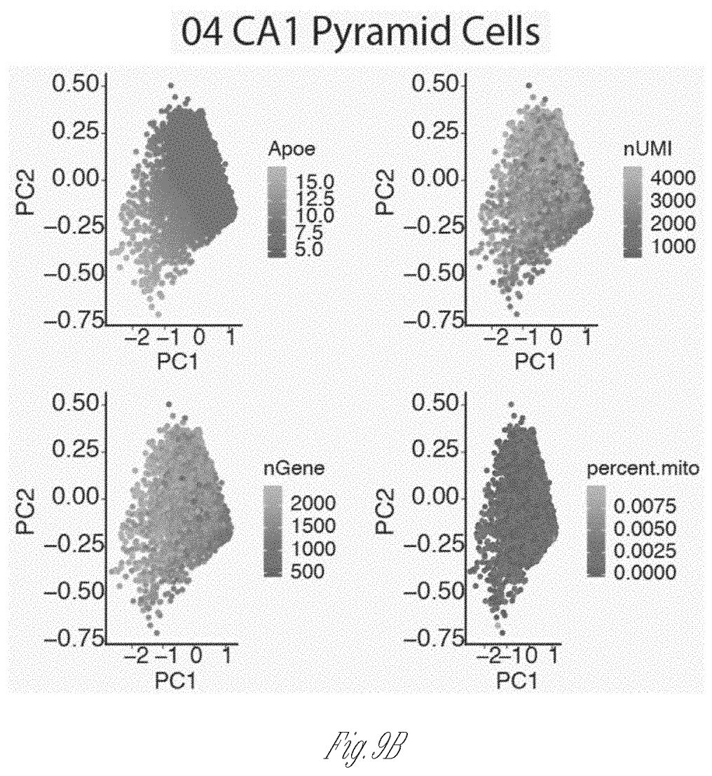

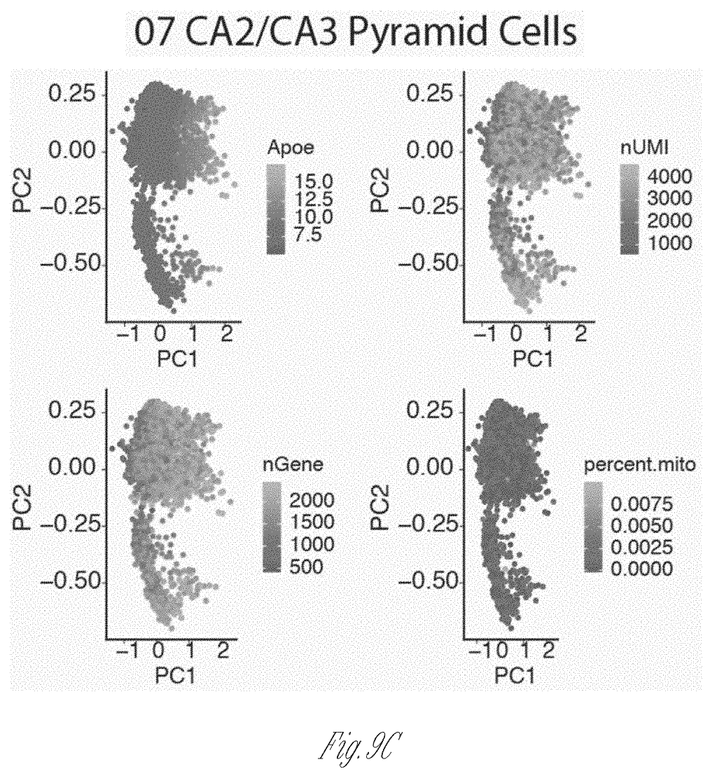

In examining the level and distribution of apoE mRNA across cell types, differences in both the median gene expression and the distribution of apoE expression across hippocampal cells types in apoE-KI mice were noticed. For example, in dentate gyms granule cells and CA1 principal cells, the median apoE expression was approximately 40% lower than that observed in SST+ and PV+ interneurons, and the median expression in SST/PV cells was less than half of that observed in astrocytes (FIG. 6A-6D). Additionally, astrocytes exhibited a strong negative skew of apoE expression, with most cells expressing a high level of apoE mRNA and a select few expressing at a low level. In contrast, the neuronal cell types uniformly exhibited a marked positive skew, with most neurons expressing a low level of apoE and a select few cells expressing apoE at a much higher level.

Again, neurons were classified as apoE-expression-high if they express apoE mRNA at more than two standard deviations above the median expression for that neuron type (red dashed lines in FIG. 6A-6D). The proportion of apoE-expression-high cells varied in an interesting way by age and apoE genotype across neuronal clusters. In both dentate gyms granule cells and CA1 pyramidal cells, it was seen that at 5 months, the proportion of apoE-expression-high cells is similar between apoE3-KI and apoE4-KI mice. Strikingly, in apoE4-KI mice, the proportion of apoE-expression-high cells rises rapidly, peaking around 10 months, before declining as the mice continue to age (FIGS. 6E and 6F). In apoE3-KI mice, a delay in this timeline was observed, with apoE-expression-high cell frequency peaking around 15 months, with a subsequent decline (FIGS. 6E and 6F). Interestingly, these timelines correlate strongly with the age of onset of neuronal and behavioral deficits in the apoE4-KI and apoE3-KI mice, respectively (Andrews-Zwilling et al., 2010; Leung et al., 2012). In SST/PV interneurons, in both apoE3-KI and apoE4-KI mice, the highest levels of apoE-expression-high cells at 5 months with subsequent decline was seen. This decline was faster and larger in apoE4-KI than in apoE3-KI mice (FIG. 6G). Again this finding aligns with previously observed timelines of GABAergic interneuron degeneration, as SST cells in the dentate gyms begin to be lost as early as 6 months in this model (Andrews-Zwilling et al., 2010; Leung et al., 2012; Li et al., 2009). These data further suggest a causal role for neuronal apoE, especially apoE4, in AD-related selective neuronal degeneration and loss. Strikingly, the apoE expression pattern in astrocytes had no such age and genotype related changes (FIG. 6H).

Neuron-Specific Knockout of the APOE Gene Protects from apoE4-Induced MHC Upregulation and Neuronal and Hippocampal Volume Loss in Aged apoE-KI Mice

To directly test the effects of neuron-specific apoE expression on neuronal health and survival, an immunohistochemical analysis of the hippocampus from aged apoE-KI mice as compared to apoE-KI mice with the APOE gene specifically knocked out of neurons (apoE-KI/Syn-Cre mice). The first phenotype that was examined was neuronal density in the CA1 region of hippocampus. It was found that aged apoE4-KI mice have a significantly lower density of NeuN/DAPI double-positive cells in CA1, as compared to apoE3-KI or apoE4-KI/Syn-Cre mice (FIGS. 6I and 6J). Interestingly, while there was no difference in neuronal density between apoE3-KI and apoE3-KI/Syn-Cre mice, knocking the APOE gene out of neurons in apoE4-KI mice rescued their CA1 neuron counts back to apoE3-KI levels (FIGS. 6I and 6J), implying that neuronal apoE4 is sufficient to cause neuronal loss by age of 16 months. Importantly, a significant negative correlation was found between neuronal density and MHC-I expression in these same neurons (FIG. 6K), again supporting a role for MHC-I in neuronal apoE-mediated selective neurodegeneration. This effect of neuronal apoE appears to be wide-ranging within the hippocampus; hippocampal volume (calculated as in (DeVos et al., 2017; Shi et al., 2017)) was significantly lower in apoE4-KI mice as compared to apoE3-KI mice (FIGS. 6L and 6M). Interestingly, in both apoE3-KI and apoE4-KI mice, this effect was significantly rescued by the neuron-specific knockout of the APOE gene (FIGS. 6L and 6M). This finding is in line with previous reports that mouse Apoe gene knockout in all cells is protective against many hallmarks of neurodegeneration, even when compared to apoE3-KI or the relatively-protective apoE2-KI backgrounds (Shi et al., 2017); but the study more specifically highlights the critical role of neuronal apoE in this process.

Neuron-Specific Knockout of the APOE Gene Protects from apoE4-Induced Neuronal MHC Increase and Aggregation as Well as Synaptic Loss in Aged apoE-KI Mice

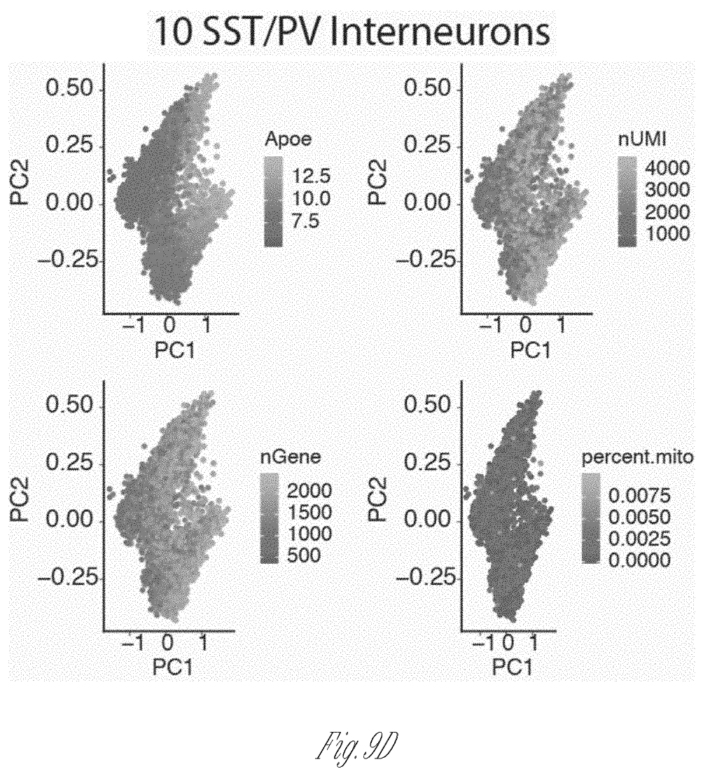

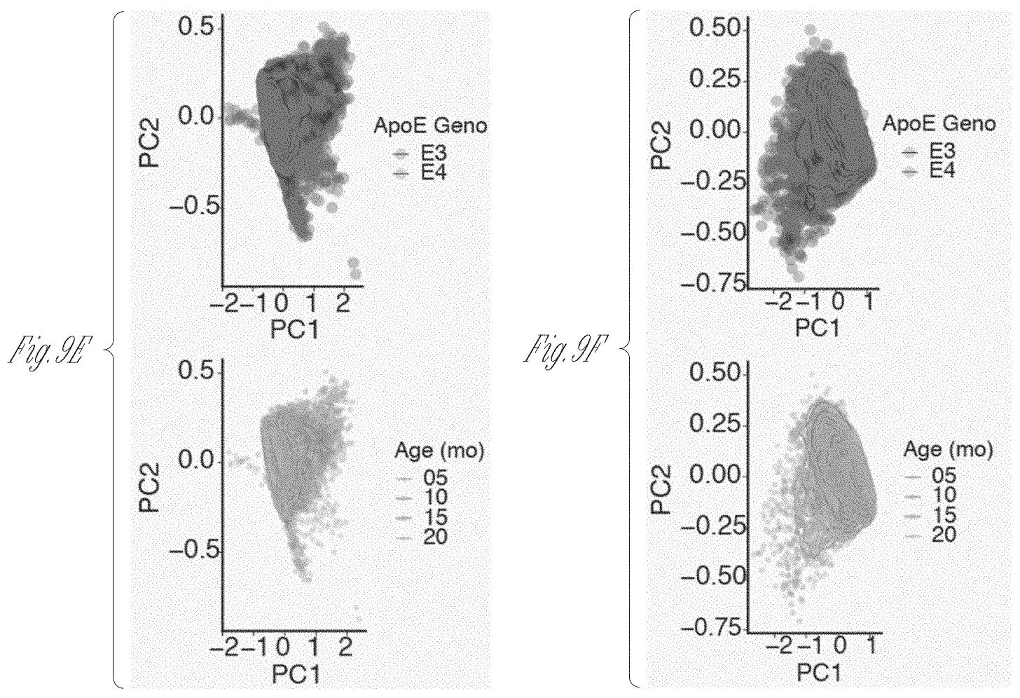

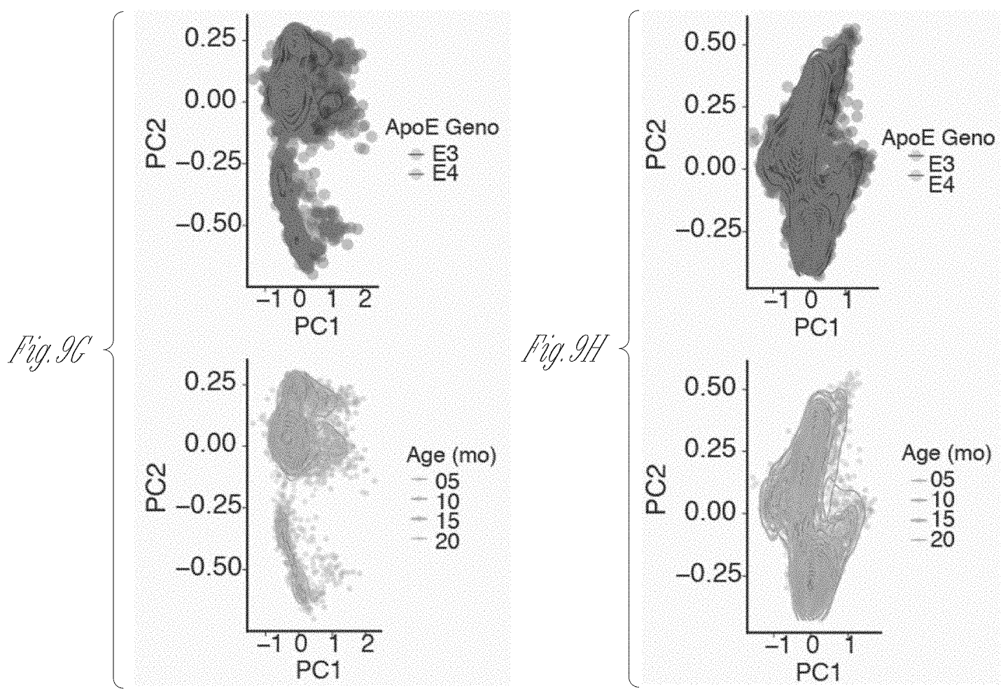

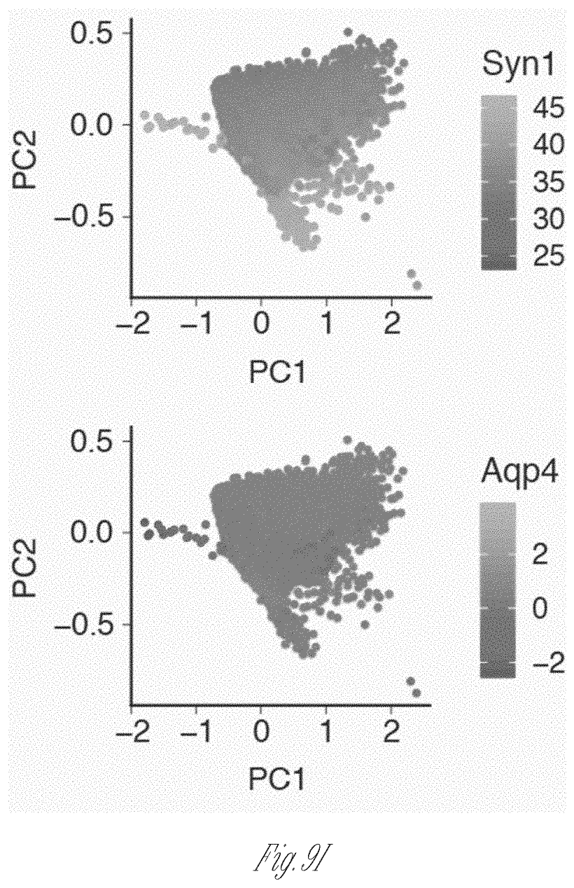

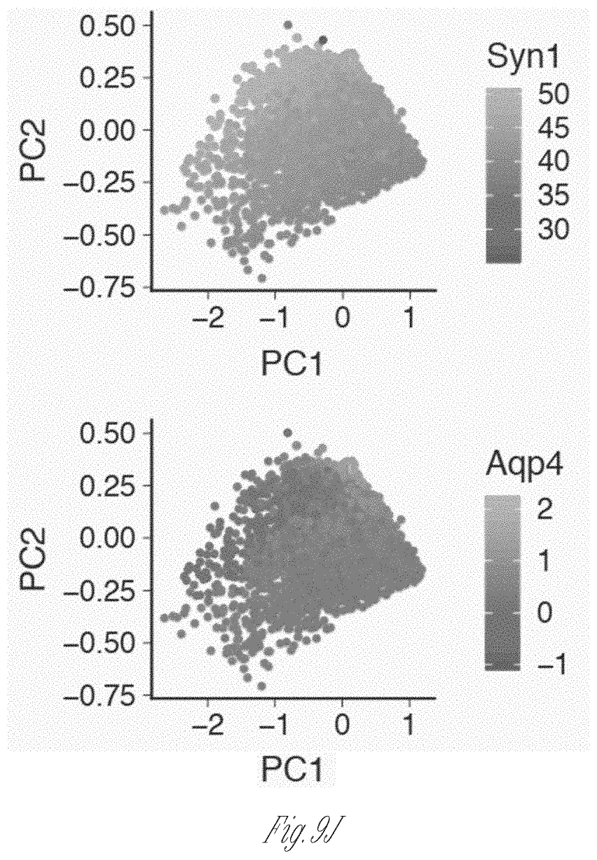

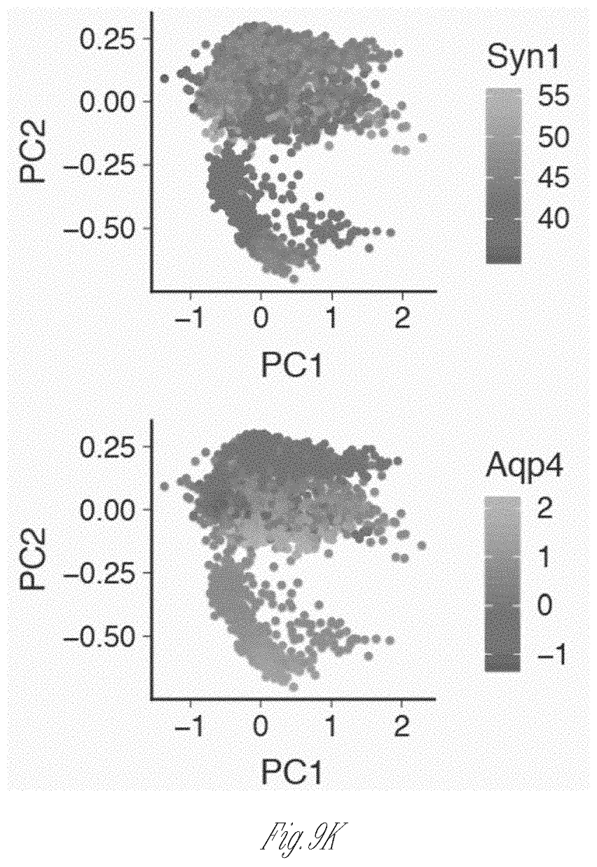

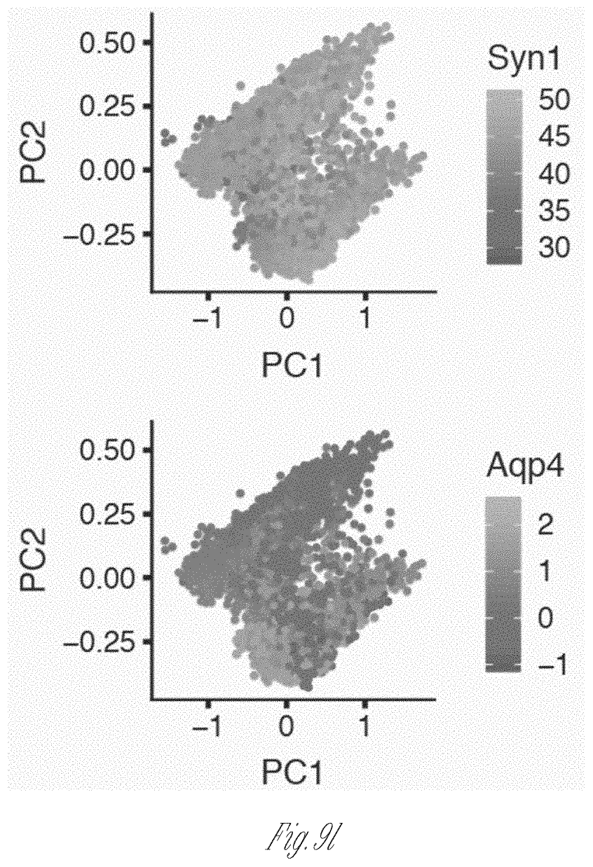

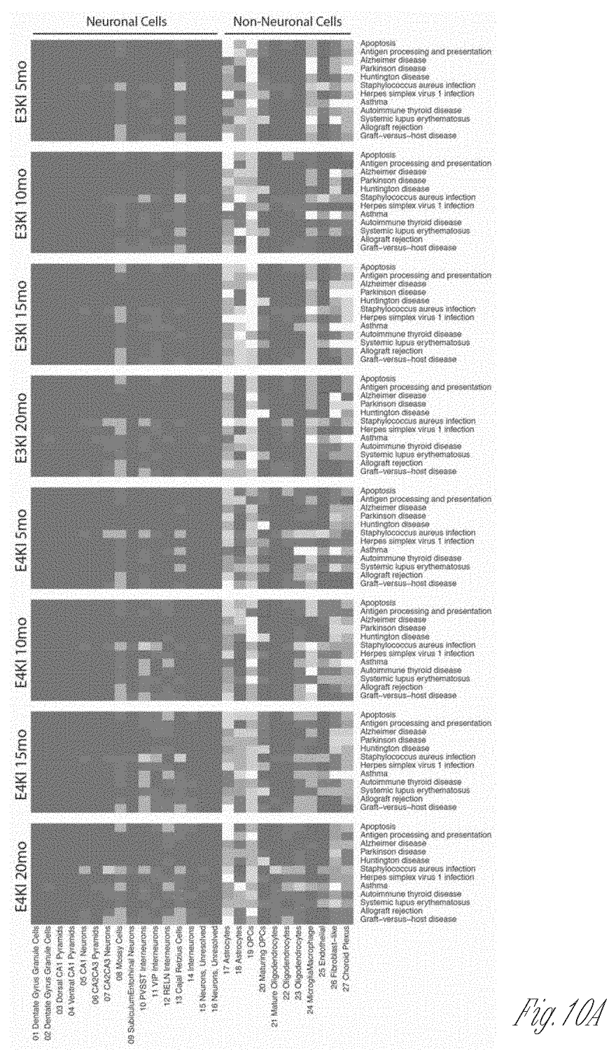

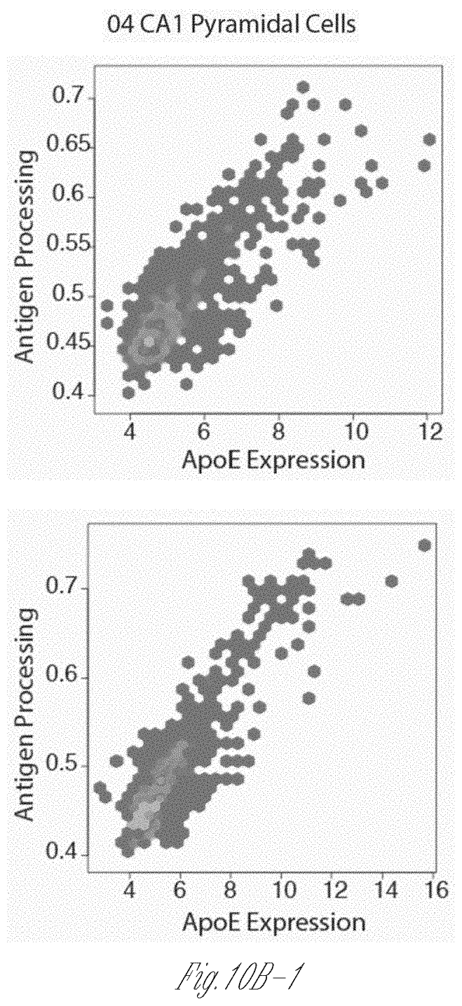

In addition to the frank neuronal loss observed in aged apoE4-KI mice, a number of synaptic phenotypes that were mediated by both neuronal apoE and MHC-I were also noted. For example, PSD-95 intensity both in cell bodies and in dendrites of CA1 was lower in aged apoE4-KI mice compared to apoE3-KI mice (FIG. 7A-7C). Additionally, both phenotypes were rescued by neuron-specific apoE knockout (FIG. 7A-7C), suggesting that neuronal apoE mediates synapse loss in apoE4-KI mice. In all mice, aggregates marked by the PSD-95 antibody that were substantially larger than the diffuse small puncta that characterize most PSD-95 staining were noted (FIG. 7A, white arrowheads). Those aggregates that were at least 2 μm in diameter were quantified and it was found that they occurred with significantly greater frequency in aged apoE4-KI mice compared to apoE3-KI mice; this phenotype too was rescued by neuron-specific apoE knockout (FIGS. 7A and 7D). Interestingly, the density of these aggregates was inversely proportional to the overall PSD-95 intensity in the dendrites of CA1 pyramidal cells (FIG. 7E), suggesting PSD-95 transport or maintenance defect mediated by neuronal apoE. It was clear that most of these PSD-95 positive aggregates were also marked by MHC-I (FIGS. 7A, yellow arrowheads, and 7F). In fact, in apoE-KI mice, ˜60% of the PSD-95 aggregates was colocalized with MHC-I, regardless of apoE genotype (FIG. 7F). In both apoE3-KI and apoE4-KI mice, this colocalization was significantly reduced by neuron-specific apoE knockout (FIG. 7F), suggesting that neuronal apoE enhances the formation of MHC-I/PSD-95 aggregates. Indeed, a direct correlation was observed between the density of PSD-95 aggregates and the density of MHC-I puncta in CA1 pyramidal cells across all mouse genotype groups (FIG. 7G). Furthermore, both the overall intensity of MHC-I staining in the CA1 pyramidal layer, and the number of MHC-I puncta per cell, were significantly higher in apoE4-KI compared to apoE3-KI or apoE4-KI/Syn-Cre mice (FIGS. 7H and 7I), again suggesting that apoE genotype, and neuronal apoE expression in particular, regulate MHC-I expression and signaling in CA1 pyramidal cells. Together, these findings strongly suggest that neuronal expression of apoE, especially apoE4, upregulates neuronal MHC expression. The increased MHC aggregates together with PSD-95 , and the aggregate levels directly correlate with the degree of synaptic and neuronal loss.

Discussion

In this study, the power of single-nucleus RNA sequencing to examine neuron-by-neuron differences in susceptibility to AD-related neurodegeneration is exploited. It is found that the most sweeping difference between individual neurons is expression of MHC pathways, and expression of these pathways is regulated by neuronal apoE expression in both mouse and human brains. In apoE-KI mice, neuron-specific apoE4 knockout reduces MHC expression and rescues synaptic, neuronal, and hippocampal volume loss. Together these data support a model where neurons under stress, due to aging, injury, excitotoxicity, infection, or accumulation of Aβ or phosphorylated tau (p-tau), upregulate apoE expression. Neuronal apoE, especially apoE4, in turn, drives neuronal MHC overexpression, which leads to selective synaptic and neuronal loss (FIG. 7J).

ApoE Upregulation of MHC Pathways in Neurons Drives Selective Neuronal and Synaptic Degeneration/Loss.

ApoE is the single biggest genetic risk factor for AD (Farrer et al., 1997; Huang and Mucke, 2012; Liu et al., 2013; Mahley and Huang, 2012), and its expression has been shown to exacerbate neurodegenerative pathologies (Brecht et al., 2004; Huang and Mucke, 2012; Huang et al., 2001; Najm et al., 2019; Shi et al., 2017; Wang et al., 2018). Likewise, both MHC-I and MHC-II have risk loci for AD (Kunkle et al., 2019; Lambert et al., 2013; Steele et al., 2017), and MHC expression is increased in AD brains (Bossers et al., 2010; Durrenberger et al., 2015), where it correlates with cognitive decline (Parachikova et al., 2007).

It has been reported that apoE and MHC are upregulated in neurons under similar conditions, are upstream effectors of related neuronal phenotypes, and are causally related to AD etiology. For example, both neuronal apoE and neuronal MHC are upregulated by stress, injury, aging, and excitotoxic insult (Adelson et al., 2016; Bombeiro et al., 2016; Corriveau et al., 1998; Huang and Mucke, 2012; Mangold et al., 2017; Najm et al., 2019; Starkey et al., 2012; Wang et al., 2018; Xu et al., 1996, 2006). Likewise, both neuronal apoE and neuronal MHC reduce neurons' capacity for synaptic plasticity, neurite outgrowth, and neuronal regeneration (Adelson et al., 2016; Datwani et al., 2009; Huang and Mucke, 2012; Kunkle et al., 2019; Najm et al., 2019; Wadhwani et al., 2019; Wang et al., 2018; Xu et al., 2006).

The work is the first to uncover a link between neuronal apoE and neuronal MHC on a cell-by-cell basis to drive selective neuronal and synaptic degeneration/loss. Critically, an upstream regulatory role for neuronal apoE on neuronal MHC expression at both the mRNA and protein levels is demonstrated.

ApoE Upregulation of MHC in Neurons may Present the Tagged Neurons to Microglia, Leading to Selective Neuronal and Synaptic Degeneration/Loss

As increased attention comes to the immune contributions to AD pathogenesis, it is important to emphasize the multi-layered, redundant, and fine regulation of the immune response. It seems necessary that neurons produce signals that attract, arrest, or activate immune cells in their niche, and highly unlikely that immune cells (resident microglia and/or brain-penetrating T-cells) would attack specific synapses or neurons without a direct signal from the neurons themselves (Brown and Neher, 2014). Indeed, there is substantial evidence for neuronal MHC serving in this capacity during development and potentially during disease (Adelson et al., 2012; Datwani et al., 2009; Huh et al., 2000; Kim et al., 2013; Lee et al., 2014). The data support the model that neuronal apoE expression, as a molecular switch, triggers aberrant upregulation of this neuronal-immune signaling pathway, driving the selective destruction of individual synapses and neurons, potentially by reactive microglia, in the AD context (FIG. 7J).

Indeed, the last decade of AD research has produced overwhelming evidence in favor of the causal role of microglia-mediated synapse and neuronal loss in AD (Hansen et al., 2018). Recent evidence indicates that a subset of microglia behave similarly to peripheral immune cells, sensing neurodegeneration-associated signals coming from dying neuronal cells, apoptotic bodies, and aberrant protein aggregates, with constitutively-expressed sensors such as TREM2 (Deczkowska et al., 2018; Wang et al., 2015). These data are in line with the hypothesis that microglia's developmental role in synaptic pruning is aberrantly re-activated in the process of aging-related neurodegeneration, leading to synaptic and neuronal loss as seen in AD (Hong et al., 2016). The study provides evidence that neuronal apoE-induced MHC overexpression serves as an “eat me” signal from stressed or injured neurons, potentially to reactive microglia (FIG. 7J).

ApoE Upregulation of MHC in Neurons May Present the Tagged Neurons to T-Cells, Leading to Selective Neuronal and Synaptic Degeneration/Loss

Microglia are not the only potential immune mediators of synaptic and neuronal loss in AD, however. Evidence is also accumulating for both microglial-dependent (Rogers et al., 1988; Schetters et al., 2018) and microglia-independent roles for brain infiltrating T-cells as well (Chevalier et al., 2011; Di Liberto et al., 2018; Dulken et al., 2019; Medana et al., 2001). Although the brain has long been thought to be an immune-privileged space, recent evidence indicates that T-cells do infiltrate the aging brain, where they release interferon-gamma (IFNγ), disrupting with the function of resident neuronal stem cells (Dulken et al., 2019). Interestingly, mere exposure to IFNγ has been shown to induce MHC-I expression in both rat and human neurons (Cebrián et al., 2014; Neumann et al., 1995). Additionally, neurons infected with neurotropic viruses, such as herpes simplex virus (HSV), increase surface expression of MHC-I, attracting CD8+ T-cells (Pereira and Simmons, 1999), which leads to T cell-mediated neurite transection (Medana et al., 2001), synaptic stripping (Di Liberto et al., 2018), and neuronal apoptosis (Chevalier et al., 2011). These findings are of particular interest given recent data indicating that HSV is more abundant in the brains of human patients with AD and directly correlates to neuronal loss (Readhead et al., 2018).

Along these lines, human APOE4 carriers have increased circulating activated T cells; APOE4 genotype together with HSV infection status more dramatically increases AD risk (Itzhaki et al., 1997); and T-cell infiltration is increased in the hippocampus (Togo et al., 2002) and CSF (Lueg et al., 2015) in AD patients, where it predicts structural MRI changes and cognitive decline (Lueg et al., 2015). T-cells have also been observed in both neuritic plaques and neurofibrillary tangles (NFTs) in AD patient brains (Rogers et al., 1988), and in AD patients, a positive correlation has been demonstrated between the number of CD3+ T cells and the degree of hyperphosphorylated tau (Zotova et al., 2013). Recent data suggest that T-cell mediated neurodegeneration may represent a common mechanism across neurodegenerative disorders, including Parkinson's disease (Brochard et al., 2009; Sulzer et al., 2017), multiple sclerosis (Hauser and Oksenberg, 2006), and narcolepsy (Bernard-Valnet et al., 2016).

In summary, the comprehensive single cell studies using both mouse and human brain samples with different apoE genotypes uncover an unknown role of neuronal apoE in regulating neuronal MHC pathways, contributing to selective synaptic and neuronal degeneration/loss in AD. This study also provides potential new targets for developing drugs to prevent or treat AD, such as lowering/blocking neuronal expression of apoE, disconnecting apoE-MHC-axis in neurons, or blocking MHC presentation of neurons to microglia and/or T-cells.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for reducing neuronal and synaptic degeneration or loss in a population of neuronal cells, the method comprising:
modulating the level and/or activity of apolipoprotein E (apoE) in the population of neuronal cells, wherein the modulating reduces the level and/or activity of at least one MI-IC pathway polypeptide in the population of neuronal cells compared to the level and/or activity of the at least one MEIC pathway polypeptide in a population of neuronal cells in the absence of said modulating.

2. The method of claim 1, wherein apoE is apoE4.

3. The method of claim 1, wherein apoE is apoE3.

4. The method of claim 1, wherein the modulating reduces the level and/or activity of apoE by at least 10%.

5. The method of claim 1, wherein the modulating reduces MHC signaling by the population of neuronal cells.

6. The method of claim 1, wherein the at least one MHC pathway polypeptide comprises Tapbp, Tap2, β2m, MHC class I, or NHC class II.

7. The method of claim 1, wherein the level and/or activity of the at least one MHC pathway polypeptide decreases by at least 10%.

8. The method of claim 1, wherein the population of neuronal cells comprises at least one of excitatory neurons, inhibitory neurons, and interneurons.

9. The method of claim 1, wherein the population of neuronal cells comprises at least one of dentate gyrus granule cells, CA1 principal cells, CA2/CA3 principal cells, subiculum/entorhinal cells, or SST/PV interneurons.

10. The method of claim 1, wherein the population of neuronal cells has been subjected to at least one of stress, injury, and aging.

11. The method of claim 1, wherein the population of neuronal cells is a population of neuronal cells in a brain region of an individual.

12. The method of claim 11, wherein the brain region is the hippocampus.

13. The method of claim 11, wherein the individual has a neurocognitive disorder or a stage thereof.

14. The method of claim 11, wherein the individual has mild cognitive impairment (MCI), prodromal Alzheimer's Disease, or Alzheimer's Disease.

15. The method of claim 1, wherein the modulating reduces loss of neuronal density in the population of neuronal cells.

16. The method of claim 1, wherein the modulating reduces loss of volume in the population of neuronal cells.

17. The method of claim 1, wherein the modulating reduces synaptic loss in the population of neuronal cells.

18. The method of claim 1, wherein the modulating reduces a decline in PSD-95 intensity in the population of neuronal cells.

19. The method of claim 1, wherein the modulating reduces PSD-95 aggregates in the population of neuronal cells.

20. The method of claim 1, wherein the modulating reduces colocalization of PSD-95 aggregates with at least one MHC pathway polypeptide in the population neuronal cells.

21-42. (canceled)

43. The method of claim 1, wherein modulating the level and/or activity of apolipoprotein E (apoE) in the population of neuronal cells comprises contacting neuronal cells in vitro or in vivo with at least one agent comprising an antibody to apoE, a small molecule, a protein inhibitor, a siRNA, a viral vector, or a CRISPR enzyme.

44. The method of claim 43, wherein the contacting comprises administering an effective amount of said at least one agent to a subject in need thereof.
